SEQUENCE LISTING (64 sequences)

SEQ ID NO: 1 - DNA, 2128 bp, Homo sapiens
  CDS (146)..(1672)
gggggtcccatcgggcccgccctcgcacgtcactccgggacccccgcggcctccgcaggttctgcgctccaggccggagtcagagactccaggatcggttctttcatcttcgccgcccctgcgcgtccagctcttctaagacgagatgccgtcgggcttccaacagataggctccgaagatggggaaccccctcagcagcgagtgactgggaccctggtccttgctgtgttctctgcggtgcttggctccctgcagtttgggtacaacattggggtcatcaatgcccctcagaaggtgattgaacagagctacaatgagacgtggctggggaggcaggggcctgagggacccagctccatccctccaggcaccctcaccaccctctgggccctctccgtggccatcttttccgtgggcggcatgatttcctccttcctcattggtatcatctctcagtggcttggaaggaaaagggccatgctggtcaacaatgtcctggcggtgctggggggcagcctcatgggcctggccaacgctgctgcctcctatgaaatgctcatccttggacgattcctcattggcgcctactcagggctgacatcagggctggtgcccatgtacgtgggggagattgctcccactcacctgcggggcgccctggggacgctcaaccaactggccattgttatcggcattctgatcgcccaggtgctgggcttggagtccctcctgggcactgccagcctgtggccactgctcctgggcctcacagtgctacctgccctcctgcagctggtcctgctgcccttctgtcccgagagcccccgctacctctacatcatccagaatctcgaggggcctgccagaaagagtctgaagcgcctgacaggctgggccgatgtttctggagtgctggctgagctgaaggatgagaagcggaagctggagcgtgagcggccactgtccctgctccagctcctgggcagccgtacccaccggcagcccctgatcattgcggtcgtgctgcagctgagccagcagctctctggcatcaatgctgttttctattattcgaccagcatcttcgagacagcaggggtaggccagcctgcctatgccaccataggagctggtgtggtcaacacagtcttcaccttggtctcggtgttgttggtggagcgggcggggcgccggacgctccatctcctgggcctggcgggcatgtgtggctgtgccatcctgatgactgtggctctgctcctgctggagcgagttccagccatgagctacgtctccattgtggccatctttggcttcgtggcattttttgagattggccctggccccattccttggttcatcgtggccgagctcttcagccagggaccccgcccggcagccatggctgtggctggtttctccaactggacgagcaacttcatcattggcatgggtttccagtatgttgcggaggctatggggccctacgtcttccttctatttgcggtcctcctgctgggcttcttcatcttcaccttcttaagagtacctgaaactcgaggccggacgtttgaccagatctcagctgccttccaccggacaccctctcttttagagcaggaggtgaaacccagcacagaacttgagtatttagggccagatgagaacgactgaggggccaggcaggggtgggagagccagctctctctacccggcccagagaccccttcctttcctctgcagcactttaaccctctcttccctattatttccgggtggaaaagaatccctgcagcctggtagaattgggaagctgggggaagggtggtctgagcaccccctcattcccctcgtgtgactctcttggattatttatgtgttgtggtttggccgtggccatcagggtgggccactctcccctccctcttccttcccccatcccctttcctccccaccttccccagactcagctccagaataccttcttcgctgctagagaagggggattggagggaagacaggtctagactttctcagtgggacaaaccagagcagagagcaggacaggagacaagaaatccagtttcccaccaccttggactcctcccacaatctgggactttcact

SEQ ID NO: 2 - protein, 509 aa, Homo sapiens
MPSGFQQIGSEDGEPPQQRVTGTLVLAVFSAVLGSLQFGYNIGVINAPQKVIEQSYNETWLGRQGPEGPSSIPPGTLTTLWALSVAIFSVGGMISSFLIGIISQWLGRKRAMLVNNVLAVLGGSLMGLANAAASYEMLILGRFLIGAYSGLTSGLVPMYVGEIAPTHLRGALGTLNQLAIVIGILIAQVLGLESLLGTASLWPLLLGLTVLPALLQLVLLPFCPESPRYLYIIQNLEGPARKSLKRLTGWADVSGVLAELKDEKRKLERERPLSLLQLLGSRTHRQPLIIAVVLQLSQQLSGINAVFYYSTSIFETAGVGQPAYATIGAGVVNTVFTLVSVLLVERAGRRTLHLLGLAGMCGCAILMTVALLLLERVPAMSYVSIVAIFGFVAFFEIGPGPIPWFIVAELFSQGPRPAAMAVAGFSNWTSNFIIGMGFQYVAEAMGPYVFLLFAVLLLGFFIFTFLRVPETRGRTFDQISAAFHRTPSLLEQEVKPSTELEYLGPDEND

SEQ ID NO: 3 - DNA, 1566 bp, Artificial sequence
  Homo sapiens GLUT4 with HA tag
  CDS (1)..(1566)
atgccgtcgggcttccaacagataggctccgaagatggggaaccccctcagcagcgagtgactgggaccctggtccttgctgtgttctctgcggtgcttggctccctgcagtttgggtacaacattggggtcatcaatgcccctcagaaggtgattgaacagagctacaatgagacgtggctggggaggcaggggcctgagatcgattatccttatgatgttcctgattatgctgagggacccagctccatccctccaggcaccctcaccaccctctgggccctctccgtggccatcttttccgtgggcggcatgatttcctccttcctcattggtatcatctctcagtggcttggaaggaaaagggccatgctggtcaacaatgtcctggcggtgctggggggcagcctcatgggcctggccaacgctgctgcctcctatgaaatgctcatccttggacgattcctcattggcgcctactcagggctgacatcagggctggtgcccatgtacgtgggggagattgctcccactcacctgcggggcgccctggggacgctcaaccaactggccattgttatcggcattctgatcgcccaggtgctgggcttggagtccctcctgggcactgccagcctgtggccactgctcctgggcctcacagtgctacctgccctcctgcagctggtcctgctgcccttctgtcccgagagcccccgctacctctacatcatccagaatctcgaggggcctgccagaaagagtctgaagcgcctgacaggctgggccgatgtttctggagtgctggctgagctgaaggatgagaagcggaagctggagcgtgagcggccactgtccctgctccagctcctgggcagccgtacccaccggcagcccctgatcattgcggtcgtgctgcagctgagccagcagctctctggcatcaatgctgttttctattattcgaccagcatcttcgagacagcaggggtaggccagcctgcctatgccaccataggagctggtgtggtcaacacagtcttcaccttggtctcggtgttgttggtggagcgggcggggcgccggacgctccatctcctgggcctggcgggcatgtgtggctgtgccatcctgatgactgtggctctgctcctgctggagcgagttccagccatgagctacgtctccattgtggccatctttggcttcgtggcattttttgagattggccctggccccattccttggttcatcgtggccgagctcttcagccagggaccccgcccggcagccatggctgtggctggtttctccaactggacgagcaacttcatcattggcatgggtttccagtatgttgcggaggctatggggccctacgtcttccttctatttgcggtcctcctgctgggcttcttcatcttcaccttcttaagagtacctgaaactcgaggccggacgtttgaccagatctcagctgccttccaccggacaccctctcttttagagcaggaggtgaaacccagcacagaacttgagtatttagggccagatgagaacgactga

SEQ ID NO: 4 - protein, 521 aa, Artificial sequence
  Synthetic Construct
MPSGFQQIGSEDGEPPQQRVTGTLVLAVFSAVLGSLQFGYNIGVINAPQKVIEQSYNETWLGRQGPEIDYPYDVPDYAEGPSSIPPGTLTTLWALSVAIFSVGGMISSFLIGIISQWLGRKRAMLVNNVLAVLGGSLMGLANAAASYEMLILGRFLIGAYSGLTSGLVPMYVGEIAPTHLRGALGTLNQLAIVIGILIAQVLGLESLLGTASLWPLLLGLTVLPALLQLVLLPFCPESPRYLYIIQNLEGPARKSLKRLTGWADVSGVLAELKDEKRKLERERPLSLLQLLGSRTHRQPLIIAVVLQLSQQLSGINAVFYYSTSIFETAGVGQPAYATIGAGVVNTVFTLVSVLLVERAGRRTLHLLGLAGMCGCAILMTVALLLLERVPAMSYVSIVAIFGFVAFFEIGPGPIPWFIVAELFSQGPRPAAMAVAGFSNWTSNFIIGMGFQYVAEAMGPYVFLLFAVLLLGFFIFTFLRVPETRGRTFDQISAAFHRTPSLLEQEVKPSTELEYLGPDEND

SEQ ID NO: 5 - protein, 512 aa, Artificial sequence
  TAIL mutant of Homo sapiens GLUT4
MPSGFQQIGSEDGEPPQQRVTGTLVLAVFSAVLGSLQFGYNIGVINAPQKVIEQSYNETWLGRQGPEIDEGPSSIPPGTLTTLWALSVAIFSVGGMISSFLIGIISQWLGRKRAMLVNNVLAVLGGSLMGLANAAASYEMLILGRFLIGAYSGLTSGLVPMYVGEIAPTHLRGALGTLNQLAIVIGILIAQVLGLESLLGTASLWPLLLGLTVLPALLQLVLLPFCPESPRYLYIIQNLEGPARKSLKRLTGWADVSGVLAELKDEKRKLERERPLSLLQLLGSRTHRQPLIIAVVLQLSQQLSGINAVFYYSTSIFETAGVGQPAYATIGAGVVNTVFTLVSVLLVERAGRRTLHLLGLAGMCGCAILMTVALLLLERVPAMSYVSIVAIFGFVAFFEIGPGPIPWFIVAELFSQGPRPAAMAVAGFSNWTSNFIIGMGFQYVAEAMGPYVFLLFAVLLLGFFIFTFLRVPETRGRTFDQISAAFHRTPSLLEQEVKPSSIEPAKETTTNV

SEQ ID NO: 6 - protein, 512 aa, Artificial sequence
  TAIL mutant of Homo sapiens GLUT4 with HA tag
MPSGFQQIGSEDGEPPQQRVTGTLVLAVFSAVLGSLQFGYNIGVINAPQKVIEQSYNETWLGRQGPEIDEGPSSIPPGTLTTLWALSVAIFSVGGMISSFLIGIISQWLGRKRAMLVNNVLAVLGGSLMGLANAAASYEMLILGRFLIGAYSGLTSGLVPMYVGEIAPTHLRGALGTLNQLAIVIGILIAQVLGLESLLGTASLWPLLLGLTVLPALLQLVLLPFCPESPRYLYIIQNLEGPARKSLKRLTGWADVSGVLAELKDEKRKLERERPLSLLQLLGSRTHRQPLIIAVVLQLSQQLSGINAVFYYSTSIFETAGVGQPAYATIGAGVVNTVFTLVSVLLVERAGRRTLHLLGLAGMCGCAILMTVALLLLERVPAMSYVSIVAIFGFVAFFEIGPGPIPWFIVAELFSQGPRPAAMAVAGFSNWTSNFIIGMGFQYVAEAMGPYVFLLFAVLLLGFFIFTFLRVPETRGRTFDQISAAFHRTPSAAEQEVKPSTELEYLGPDEND

SEQ ID NO: 7 - protein, 521 aa, Artificial sequence
  L489,490A mutant of Homo sapiens GLUT4 with HA tag
MPSGFQQIGSEDGEPPQQRVTGTLVLAVFSAVLGSLQFGYNIGVINAPQKVIEQSYNETWLGRQGPEIDYPYDVPDYAEGPSSIPPGTLTTLWALSVAIFSVGGMISSFLIGIISQWLGRKRAMLVNNVLAVLGGSLMGLANAAASYEMLILGRFLIGAYSGLTSGLVPMYVGEIAPTHLRGALGTLNQLAIVIGILIAQVLGLESLLGTASLWPLLLGLTVLPALLQLVLLPFCPESPRYLYIIQNLEGPARKSLKRLTGWADVSGVLAELKDEKRKLERERPLSLLQLLGSRTHRQPLIIAVVLQLSQQLSGINAVFYYSTSIFETAGVGQPAYATIGAGVVNTVFTLVSVLLVERAGRRTLHLLGLAGMCGCAILMTVALLLLERVPAMSYVSIVAIFGFVAFFEIGPGPIPWFIVAELFSQGPRPAAMAVAGFSNWTSNFIIGMGFQYVAEAMGPYVFLLFAVLLLGFFIFTFLRVPETRGRTFDQISAAFHRTPSAAEQEVKPSTELEYLGPDEND

SEQ ID NO: 8 - protein, 512 aa, Artificial sequence
  F5A mutant of Homo sapiens GLUT4
MPSGAQQIGSEDGEPPQQRVTGTLVLAVFSAVLGSLQFGYNIGVINAPQKVIEQSYNETWLGRQGPEIDEGPSSIPPGTLTTLWALSVAIFSVGGMISSFLIGIISQWLGRKRAMLVNNVLAVLGGSLMGLANAAASYEMLILGRFLIGAYSGLTSGLVPMYVGEIAPTHLRGALGTLNQLAIVIGILIAQVLGLESLLGTASLWPLLLGLTVLPALLQLVLLPFCPESPRYLYIIQNLEGPARKSLKRLTGWADVSGVLAELKDEKRKLERERPLSLLQLLGSRTHRQPLIIAVVLQLSQQLSGINAVFYYSTSIFETAGVGQPAYATIGAGVVNTVFTLVSVLLVERAGRRTLHLLGLAGMCGCAILMTVALLLLERVPAMSYVSIVAIFGFVAFFEIGPGPIPWFIVAELFSQGPRPAAMAVAGFSNWTSNFIIGMGFQYVAEAMGPYVFLLFAVLLLGFFIFTFLRVPETRGRTFDQISAAFHRTPSLLEQEVKPSTELEYLGPDEND

SEQ ID NO: 9 - protein, 521 aa, Artificial sequence
  F5A mutant of Homo sapiens with HA tag
MPSGAQQIGSEDGEPPQQRVTGTLVLAVFSAVLGSLQFGYNIGVINAPQKVIEQSYNETWLGRQGPEIDYPYDVPDYAEGPSSIPPGTLTTLWALSVAIFSVGGMISSFLIGIISQWLGRKRAMLVNNVLAVLGGSLMGLANAAASYEMLILGRFLIGAYSGLTSGLVPMYVGEIAPTHLRGALGTLNQLAIVIGILIAQVLGLESLLGTASLWPLLLGLTVLPALLQLVLLPFCPESPRYLYIIQNLEGPARKSLKRLTGWADVSGVLAELKDEKRKLERERPLSLLQLLGSRTHRQPLIIAVVLQLSQQLSGINAVFYYSTSIFETAGVGQPAYATIGAGVVNTVFTLVSVLLVERAGRRTLHLLGLAGMCGCAILMTVALLLLERVPAMSYVSIVAIFGFVAFFEIGPGPIPWFIVAELFSQGPRPAAMAVAGFSNWTSNFIIGMGFQYVAEAMGPYVFLLFAVLLLGFFIFTFLRVPETRGRTFDQISAAFHRTPSLLEQEVKPSTELEYLGPDEND

SEQ ID NO: 10 - DNA, 2856 bp, Homo sapiens
  CDS (180)..(1658)
tagtcgcgggtccccgagtgagcacgccagggagcaggagaccaaacgacgggggtcggagtcagagtcgcagtgggagtccccggaccggagcacgagcctgagcgggagagcgccgctcgcacgcccgtcgccacccgcgtacccggcgcagccagagccaccagcgcagcgctgccatggagcccagcagcaagaagctgacgggtcgcctcatgctggctgtgggaggagcagtgcttggctccctgcagtttggctacaacactggagtcatcaatgccccccagaaggtgatcgaggagttctacaaccagacatgggtccaccgctatggggagagcatcctgcccaccacgctcaccacgctctggtccctctcagtggccatcttttctgttgggggcatgattggctccttctctgtgggccttttcgttaaccgctttggccggcggaattcaatgctgatgatgaacctgctggccttcgtgtccgccgtgctcatgggcttctcgaaactgggcaagtcctttgagatgctgatcctgggccgcttcatcatcggtgtgtactgcggcctgaccacaggcttcgtgcccatgtatgtgggtgaagtgtcacccacagcctttcgtggggccctgggcaccctgcaccagctgggcatcgtcgtcggcatcctcatcgcccaggtgttcggcctggactccatcatgggcaacaaggacctgtggcccctgctgctgagcatcatcttcatcccggccctgctgcagtgcatcgtgctgcccttctgccccgagagtccccgcttcctgctcatcaaccgcaacgaggagaaccgggccaagagtgtgctaaagaagctgcgcgggacagctgacgtgacccatgacctgcaggagatgaaggaagagagtcggcagatgatgcgggagaagaaggtcaccatcctggagctgttccgctcccccgcctaccgccagcccatcctcatcgctgtggtgctgcagctgtcccagcagctgtctggcatcaacgctgtcttctattactccacgagcatcttcgagaaggcgggggtgcagcagcctgtgtatgccaccattggctccggtatcgtcaacacggccttcactgtcgtgtcgctgtttgtggtggagcgagcaggccggcggaccctgcacctcataggcctcgctggcatggcgggttgtgccatactcatgaccatcgcgctagcactgctggagcagctaccctggatgtcctatctgagcatcgtggccatctttggctttgtggccttctttgaagtgggtcctggccccatcccatggttcatcgtggctgaactcttcagccagggtccacgtccagctgccattgccgttgcaggcttctccaactggacctcaaatttcattgtgggcatgtgcttccagtatgtggagcaactgtgtggtccctacgtcttcatcatcttcactgtgctcctggttctgttcttcatcttcacctacttcaaagttcctgagactaaaggccggaccttcgatgagatcgcttccggcttccggcaggggggagccagccaaagtgataagacacccgaggagctgttccatcccctgggggctgattcccaagtgtgagtcgccccagatcaccagcccggcctgctcccagcagccctaaggatctctcaggagcacaggcagctggatgagacttccaaacctgacagatgtcagccgagccgggcctggggctcctttctccagccagcaatgatgtccagaagaatattcaggacttaacggctccaggattttaacaaaagcaagactgttgctcaaatctattcagacaagcaacaggttttataatttttttattactgattttgttatttttatatcagcctgagtctcctgtgcccacatcccaggcttcaccctgaatggttccatgcctgagggtggagactaagccctgtcgagacacttgccttcttcacccagctaatctgtagggctggacctatgtcctaaggacacactaatcgaactatgaactacaaagcttctatcccaggaggtggctatggccacccgttctgctggcctggatctccccactctaggggtcaggctccattaggatttgccccttcccatctcttcctacccaaccactcaaattaatctttctttacctgagaccagttgggagcactggagtgcagggaggagaggggaagggccagtctgggctgccgggttctagtctcctttgcactgagggccacactattaccatgagaagagggcctgtgggagcctgcaaactcactgctcaagaagacatggagactcctgccctgttgtgtatagatgcaagatatttatatatatttttggttgtcaatattaaatacagacactaagttatagtatatctggacaagccaacttgtaaatacaccacctcactcctgttacttacctaaacagatataaatggctggtttttagaaacatggttttgaaatgcttgtggattgagggtaggaggtttggatgggagtgagacagaagtaagtggggttgcaaccactgcaacggcttagacttcgactcaggatccagtcccttacacgtacctctcatcagtgtcctcttgctcaaaaatctgtttgatccctgttacccagagaatatatacattctttatcttgacattcaaggcatttctatcacatatttgatagttggtgttcaaaaaaacactagttttgtgccagccgtgatgctcaggcttgaaatcgcattattttgaatgtgaagggaa

SEQ ID NO: 11 - protein, 492 aa, Homo sapiens
MEPSSKKLTGRLMLAVGGAVLGSLQFGYNTGVINAPQKVIEEFYNQTWVHRYGESILPTTLTTLWSLSVAIFSVGGMIGSFSVGLFVNRFGRRNSMLMMNLLAFVSAVLMGFSKLGKSFEMLILGRFIIGVYCGLTTGFVPMYVGEVSPTAFRGALGTLHQLGIVVGILIAQVFGLDSIMGNKDLWPLLLSIIFIPALLQCIVLPFCPESPRFLLINRNEENRAKSVLKKLRGTADVTHDLQEMKEESRQMMREKKVTILELFRSPAYRQPILIAVVLQLSQQLSGINAVFYYSTSIFEKAGVQQPVYATIGSGIVNTAFTVVSLFVVERAGRRTLHLIGLAGMAGCAILMTIALALLEQLPWMSYLSIVAIFGFVAFFEVGPGPIPWFIVAELFSQGPRPAAIAVAGFSNWTSNFIVGMCFQYVEQLCGPYVFIIFTVLLVLFFIFTYFKVPETKGRTFDEIASGFRQGGASQSDKTPEELFHPLGADSQV

SEQ ID NO: 12 - DNA, 1506 bp, Artificial sequence
  Homo sapiens GLUT1 with HA tag
  CDS (1)..(1506)
atggagcccagcagcaagaagctgacgggtcgcctcatgctggctgtgggaggagcagtgcttggctccctgcagtttggctacaacactggagtcatcaatgccccccagaaggtgatcgaggagttctacaaccagacatgggtccaccgctatggggagagcatctacccatacgacgtcccagactacgctctgcccaccacgctcaccacgctctggtccctctcagtggccatcttttctgttgggggcatgattggctccttctctgtgggccttttcgttaaccgctttggccggcggaattcaatgctgatgatgaacctgctggccttcgtgtccgccgtgctcatgggcttctcgaaactgggcaagtcctttgagatgctgatcctgggccgcttcatcatcggtgtgtactgcggcctgaccacaggcttcgtgcccatgtatgtgggtgaagtgtcacccacagcctttcgtggggccctgggcaccctgcaccagctgggcatcgtcgtcggcatcctcatcgcccaggtgttcggcctggactccatcatgggcaacaaggacctgtggcccctgctgctgagcatcatcttcatcccggccctgctgcagtgcatcgtgctgcccttctgccccgagagtccccgcttcctgctcatcaaccgcaacgaggagaaccgggccaagagtgtgctaaagaagctgcgcgggacagctgacgtgacccatgacctgcaggagatgaaggaagagagtcggcagatgatgcgggagaagaaggtcaccatcctggagctgttccgctcccccgcctaccgccagcccatcctcatcgctgtggtgctgcagctgtcccagcagctgtctggcatcaacgctgtcttctattactccacgagcatcttcgagaaggcgggggtgcagcagcctgtgtatgccaccattggctccggtatcgtcaacacggccttcactgtcgtgtcgctgtttgtggtggagcgagcaggccggcggaccctgcacctcataggcctcgctggcatggcgggttgtgccatactcatgaccatcgcgctagcactgctggagcagctaccctggatgtcctatctgagcatcgtggccatctttggctttgtggccttctttgaagtgggtcctggccccatcccatggttcatcgtggctgaactcttcagccagggtccacgtccagctgccattgccgttgcaggcttctccaactggacctcaaatttcattgtgggcatgtgcttccagtatgtggagcaactgtgtggtccctacgtcttcatcatcttcactgtgctcctggttctgttcttcatcttcacctacttcaaagttcctgagactaaaggccggaccttcgatgagatcgcttccggcttccggcaggggggagccagccaaagtgataagacacccgaggagctgttccatcccctgggggctgattcccaagtgtga

SEQ ID NO: 13 - protein, 501 aa, Artificial sequence
  Synthetic Construct
MEPSSKKLTGRLMLAVGGAVLGSLQFGYNTGVINAPQKVIEEFYNQTWVHRYGESIYPYDVPDYALPTTLTTLWSLSVAIFSVGGMIGSFSVGLFVNRFGRRNSMLMMNLLAFVSAVLMGFSKLGKSFEMLILGRFIIGVYCGLTTGFVPMYVGEVSPTAFRGALGTLHQLGIVVGILIAQVFGLDSIMGNKDLWPLLLSIIFIPALLQCIVLPFCPESPRFLLINRNEENRAKSVLKKLRGTADVTHDLQEMKEESRQMMREKKVTILELFRSPAYRQPILIAVVLQLSQQLSGINAVFYYSTSIFEKAGVQQPVYATIGSGIVNTAFTVVSLFVVERAGRRTLHLIGLAGMAGCAILMTIALALLEQLPWMSYLSIVAIFGFVAFFEVGPGPIPWFIVAELFSQGPRPAAIAVAGFSNWTSNFIVGMCFQYVEQLCGPYVFIIFTVLLVLFFIFTYFKVPETKGRTFDEIASGFRQGGASQSDKTPEELFHPLGADSQV

SEQ ID NO: 14 - protein, 9 aa, Artificial sequence
  HA epitope
YPYDVPDYA

SEQ ID NO: 15 - protein, 14 aa, Artificial sequence
  Simian Virus 5 epitope (SV5)
GKPIPNPLLGLDST

SEQ ID NO: 16 - protein, 6 aa, Artificial sequence
  hexa-his
HHHHHH

SEQ ID NO: 17 - protein, 10 aa, Artificial sequence
  c-myc epitope
FQKLISEEDL

SEQ ID NO: 18 - protein, 9 aa, Artificial sequence
  FLAG epitope
DYKDDDDKC

SEQ ID NO: 19 - protein, 9 aa, Artificial sequence
  Alternative FLAG epitope
MDFKDDDDK

SEQ ID NO: 20 - protein, 9 aa, Artificial sequence
  Alternative FLAG epitope
MDYKAFDNL

SEQ ID NO: 21 - protein, 223 aa, Artificial sequence
  glutathione-S-transferase
MAKLPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDL

SEQ ID NO: 22 - protein, 488 aa, Artificial sequence
  Maltose binding protein
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIDTTENLYFQGAMDPEFKGLRRRAQLVRPLSNLEPAVSRHAVPSLALAVVLQRRDWENPGVTQLNRLAAHPPFASWRNSEEARTDRPSQQLRSLNGEWQLGCFGG

SEQ ID NO: 23 - protein, 168 aa, Artificial sequence
  GAL4
MKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSPEFPGIRRLDALISSRAAAGT

SEQ ID NO: 24 - protein, 1045 aa, Artificial sequence
  Beta-galactosidase
MSFTLTNKNVIFVAGLGGIGLDTSKELLKRDPVVLQRRDWENPGVTQLNRLAAHPPFASWRNSEEARTDRPSQQLRSLNGEWRFAWFPAPEAVPESWLECDLPEADTVVVPSNWQMHGYDAPIYTNVTYPITVNPPFVPTENPTGCYSLTFNVDESWLQEGQTRIIFDGVNSAFHLWCNGRWVGYGQDSRLPSEFDLSAFLRAGENRLAVMVLRWSDGSYLEDQDMWRMSGIFRDVSLLHKPTTQISDFHVATRFNDDFSRAVLEAEVQMCGELRDYLRVTVSLWQGETQVASGTAPFGGEIIDERGGYADRVTLRLNVENPKLWSAEIPNLYRAVVELHTADGTLIEAEACDVGFREVRIENGLLLLNGKPLLIRGVNRHEHHPLHGQVMDEQTMVQDILLMKQNNFNAVRCSHYPNHPLWYTLCDRYGLYVVDEANIETHGMVPMNRLTDDPRWLPAMSERVTRMVQRDRNHPSVIIWSLGNESGHGANHDALYRWIKSVDPSRPVQYEGGGADTTATDIICPMYARVDEDQPFPAVPKWSIKKWLSLPGETRPLILCEYAHAMGNSLGGFAKYWQAFRQYPRLQGGFVWDWVDQSLIKYDENGNPWSAYGGDFGDTPNDRQFCMNGLVFADRTPHPALTEAKHQQQFFQFRLSGQTIEVTSEYLFRHSDNELLHWMVALDGKPLASGEVPLDVAPQGKQLIELPELPQPESAGQLWLTVRVVQPNATAWSEAGHISAWQQWRLAENLSVTLPAASHAIPHLTTSEMDFCIELGNKRWQFNRQSGFLSQMWIGDKKQLLTPLRDQFTRAPLDNDIGVSEATRIDPNAWVERWKAAGHYQAEAALLQCTADTLADAVLITTAHAWQHQGKTLFISRKTYRIDGSGQMAITVDVEVASDTPHPARIGLNCQLAQVAERVNWLGLGPQENYPDRLTAACFDRWDLPLSDMYTPYVFPSENGLRCGTRELNYGPHQWRGDFQFNISRYSQQQLMSHRHLLHAEEGTWLNIDGFHMGIGGDDSWSPSVSAELQLSAGRYHYQLVWCQK

SEQ ID NO: 25 - protein, 238 aa, Artificial sequence
  enhanced green fluorescence protein (eGFP)
MSKGEELFTGVVPILVELDGDVNGHKFSVSGEGEGDATYGKLTLKFICTTGKLPVPWPTLVTTFGYGVQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 26 - protein, 264 aa, Artificial sequence
  Yellow fluorescent protein
MDGTELGSTRDSRGSGGSGGSGGSGMVSKGEELFTGVVPILVELDGDVNGHKFSVSGEGEGDATYGKLTLKFICTTGKLPVPWPTLVTTFGYGLQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYK

SEQ ID NO: 27 - protein, 238 aa, Artificial sequence
  soluble modified blue fluorescent protein
MSKGEELFTGVVPILVELDGDVNGHKFSVSGEGEGDATYGKLTLKFICTTGKLPVPWPTLVTTFSHGVQCFSRYPDHMKRHDFFKSAMPEGYVQERTISFKDDGNYKTRAEVKFEGDTLVNRIELKGIDFKEDGNILGHKLEYNYNSHNVYITADKQKNGIKANFKIRHNIEDGSVQLADHYQQNTPIGDGPVLLPDNHYLSTQSALSKDPNEKRDHMVLLEFVTAAGITHGMDELYK

SEQ ID NO: 28 - protein, 238 aa, Artificial sequence
  soluble-modified red-shifted green fluorescent protein
MSKGEELFTGVVPILVELDGDVNGHKFSVSGEGEGDATYGKLTLKFICTTGKLPVPWPTLVTTFTYGVQCFSRYPDHMKRHDFFKSAMPEGYVQERTISFKDDGNYKTRAEVKFEGDTLVNRIELKGIDFKEDGNILGHKLEYNYNSHNVYITADKQKNGIKANFKIRHNIEDGSVQLADHYQQNTPIGDGPVLLPDNHYLSTQSALSKDPNEKRDHMVLLEFVTAAGITHGMDELYK

SEQ ID NO: 29 - protein, 262 aa, Artificial sequence
  cyan fluorescent protein
MHHHHHHHDGTMVSKGEELFTGVVPILVELDGDVNGHKFSVSGEGEGDATYGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKANFKIRHNIEDGSVQLADHYQQNTPIGDGPVLLPDNHYLSTQSALSKDPNEKRDHMVLLEFVTAAGITLGMDELYSGSGSGSLEGTEL

SEQ ID NO: 30 - protein, 8 aa, Artificial sequence
  streptavidin binding sequence
WSHPQFEK

SEQ ID NO: 31 - protein, 574 aa, Artificial sequence
  strepsolysin-O
MKDMSNKKTFKKYSRVAGLLTAALIIGNLVTANAESNKQNTASTETTTTNEQPKPESSELTTEKAGQKTDDMLNSNDMIKLAPKEMPLESAEKEEKKSEDKKKSEEDHTEEINDKIYSLNYNELEVLAKNGETIENFVPKEGVKKADKFIVIERKKKNINTTPVDISIIDSVTDRTYPAALQLANKGFTENKPDAVVTKRNPQKIHIDLPGMGDKATVEVNDPTYANVSTAIDNLVNQWHDNYSGGNTLPARTQYTESMVYSKSQIEAALNVNSKILDGTLGIDFKSISKGEKKVMIAAYKQIFYTVSANLPNNPADVFDKSVTFKELQRKGVSNEAPPLFVSNVAYGRTVFVKLETSSKSNDVEAAFSAALKGTDVKTNGKYSDILENSSFTAVVLGGDAAEHNKVVTKDFDVIRNVIKDNATFSRKNPAYPISYTSVFLKNNKIAGVNNRTEYVETTSTEYTSGKINLSHRGAYVAQYEILWDEINYDDKGKEVITKRRWDNNWYSKTSPFSTVIPLGANSRNIRIMARECTGLAWEWWRKVIDERDVKLSKEINVNISGSTLSPYGSITYK

SEQ ID NO: 32 - protein, 293 aa, Artificial sequence
  alpha-hemolysin
ADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 33 - protein, 527 aa, Artificial sequence
  tetanolysin-O
MNKNVLKFVSRSLLIFSMTGLISNYNSSNVLAKGNVEEHSLINNGQVVTSNTKCNLAKDNSSDIDKNIYGLSYDPRKILSYNGEQVENFVPAEGFENPDKFIVVKREKKSISDSTADISIIDSINDRTYPGAIQLANRNLMENKPDIISCERKPITISVDLPGMAEDGKKVVNSPTYSSVNSAINSILDTWNSKYSSKYTIPTRMSYSDTMVYSQSQLSAAVGCNFKALNKALNIDFDSIFKGEKKVMLLAYKQIFYTVSVDPPNRPSDLFGDSVTFDELALKGINNNNPPAYVSNVAYGRTIYVKLETTSKSSHVKAAFKALINNQDISSNAEYKDILNQSSFTATVLGGGAQEHNKIITKDFDEIRNIIKNNSVYSPQNPGYPISYTTTFLKDNSIASVNNKTEYIETTATEYTNGKIVLDHSGAYVAQFQVTWDEVSYDEKGNEIVEHKAWEGNNRDRTAHFNTEIYLKGNARNISVKIRECTGLAWEWWRTIVDVKNIPLAKERTFYIWGTTLYPKTSIETKM

SEQ ID NO: 34 - DNA, 6129 bp, Homo sapiens
  CDS (133)..(4575)
aattggaagcaaatgacatcacagcaggtcagagaaaaagggttgagcggcaggcacccagagtagtaggtctttggcattaggagcttgagcccagacggccctagcagggaccccagcgcccgagagaccatgcagaggtcgcctctggaaaaggccagcgttgtctccaaactttttttcagctggaccagaccaattttgaggaaaggatacagacagcgcctggaattgtcagacatataccaaatcccttctgttgattctgctgacaatctatctgaaaaattggaaagagaatgggatagagagctggcttcaaagaaaaatcctaaactcattaatgcccttcggcgatgttttttctggagatttatgttctatggaatctttttatatttaggggaagtcaccaaagcagtacagcctctcttactgggaagaatcatagcttcctatgacccggataacaaggaggaacgctctatcgcgatttatctaggcataggcttatgccttctctttattgtgaggacactgctcctacacccagccatttttggccttcatcacattggaatgcagatgagaatagctatgtttagtttgatttataagaagactttaaagctgtcaagccgtgttctagataaaataagtattggacaacttgttagtctcctttccaacaacctgaacaaatttgatgaaggacttgcattggcacatttcgtgtggatcgctcctttgcaagtggcactcctcatggggctaatctgggagttgttacaggcgtctgccttctgtggacttggtttcctgatagtccttgccctttttcaggctgggctagggagaatgatgatgaagtacagagatcagagagctgggaagatcagtgaaagacttgtgattacctcagaaatgattgaaaatatccaatctgttaaggcatactgctgggaagaagcaatggaaaaaatgattgaaaacttaagacaaacagaactgaaactgactcggaaggcagcctatgtgagatacttcaatagctcagccttcttcttctcagggttctttgtggtgtttttatctgtgcttccctatgcactaatcaaaggaatcatcctccggaaaatattcaccaccatctcattctgcattgttctgcgcatggcggtcactcggcaatttccctgggctgtacaaacatggtatgactctcttggagcaataaacaaaatacaggatttcttacaaaagcaagaatataagacattggaatataacttaacgactacagaagtagtgatggagaatgtaacagccttctgggaggagggatttggggaattatttgagaaagcaaaacaaaacaataacaatagaaaaacttctaatggtgatgacagcctcttcttcagtaatttctcacttcttggtactcctgtcctgaaagatattaatttcaagatagaaagaggacagttgttggcggttgctggatccactggagcaggcaagacttcacttctaatgatgattatgggagaactggagccttcagagggtaaaattaagcacagtggaagaatttcattctgttctcagttttcctggattatgcctggcaccattaaagaaaatatcatctttggtgtttcctatgatgaatatagatacagaagcgtcatcaaagcatgccaactagaagaggacatctccaagtttgcagagaaagacaatatagttcttggagaaggtggaatcacactgagtggaggtcaacgagcaagaatttctttagcaagagcagtatacaaagatgctgatttgtatttattagactctccttttggatacctagatgttttaacagaaaaagaaatatttgaaagctgtgtctgtaaactgatggctaacaaaactaggattttggtcacttctaaaatggaacatttaaagaaagctgacaaaatattaattttgaatgaaggtagcagctatttttatgggacattttcagaactccaaaatctacagccagactttagctcaaaactcatgggatgtgattctttcgaccaatttagtgcagaaagaagaaattcaatcctaactgagaccttacaccgtttctcattagaaggagatgctcctgtctcctggacagaaacaaaaaaacaatcttttaaacagactggagagtttggggaaaaaaggaagaattctattctcaatccaatcaactctatacgaaaattttccattgtgcaaaagactcccttacaaatgaatggcatcgaagaggattctgatgagcctttagagagaaggctgtccttagtaccagattctgagcagggagaggcgatactgcctcgcatcagcgtgatcagcactggccccacgcttcaggcacgaaggaggcagtctgtcctgaacctgatgacacactcagttaaccaaggtcagaacattcaccgaaagacaacagcatccacacgaaaagtgtcactggcccctcaggcaaacttgactgaactggatatatattcaagaaggttatctcaagaaactggcttggaaataagtgaagaaattaacgaagaagacttaaaggagtgcctttttgatgatatggagagcataccagcagtgactacatggaacacataccttcgatatattactgtccacaagagcttaatttttgtgctaatttggtgcttagtaatttttctggcagaggtggctgcttctttggttgtgctgtggctccttggaaacactcctcttcaagacaaagggaatagtactcatagtagaaataacagctatgcagtgattatcaccagcaccagttcgtattatgtgttttacatttacgtgggagtagccgacactttgcttgctatgggattcttcagaggtctaccactggtgcatactctaatcacagtgtcgaaaattttacaccacaaaatgttacattctgttcttcaagcacctatgtcaaccctcaacacgttgaaagcaggtgggattcttaatagattctccaaagatatagcaattttggatgaccttctgcctcttaccatatttgacttcatccagttgttattaattgtgattggagctatagcagttgtcgcagttttacaaccctacatctttgttgcaacagtgccagtgatagtggcttttattatgttgagagcatatttcctccaaacctcacagcaactcaaacaactggaatctgaaggcaggagtccaattttcactcatcttgttacaagcttaaaaggactatggacacttcgtgccttcggacggcagccttactttgaaactctgttccacaaagctctgaatttacatactgccaactggttcttgtacctgtcaacactgcgctggttccaaatgagaatagaaatgatttttgtcatcttcttcattgctgttaccttcatttccattttaacaacaggagaaggagaaggaagagttggtattatcctgactttagccatgaatatcatgagtacattgcagtgggctgtaaactccagcatagatgtggatagcttgatgcgatctgtgagccgagtctttaagttcattgacatgccaacagaaggtaaacctaccaagtcaaccaaaccatacaagaatggccaactctcgaaagttatgattattgagaattcacacgtgaagaaagatgacatctggccctcagggggccaaatgactgtcaaagatctcacagcaaaatacacagaaggtggaaatgccatattagagaacatttccttctcaataagtcctggccagagggtgggcctcttgggaagaactggatcagggaagagtactttgttatcagcttttttgagactactgaacactgaaggagaaatccagatcgatggtgtgtcttgggattcaataactttgcaacagtggaggaaagcctttggagtgataccacagaaagtatttattttttctggaacatttagaaaaaacttggatccctatgaacagtggagtgatcaagaaatatggaaagttgcagatgaggttgggctcagatctgtgatagaacagtttcctgggaagcttgactttgtccttgtggatgggggctgtgtcctaagccatggccacaagcagttgatgtgcttggctagatctgttctcagtaaggcgaagatcttgctgcttgatgaacccagtgctcatttggatccagtaacataccaaataattagaagaactctaaaacaagcatttgctgattgcacagtaattctctgtgaacacaggatagaagcaatgctggaatgccaacaatttttggtcatagaagagaacaaagtgcggcagtacgattccatccagaaactgctgaacgagaggagcctcttccggcaagccatcagcccctccgacagggtgaagctctttccccaccggaactcaagcaagtgcaagtctaagccccagattgctgctctgaaagaggagacagaagaagaggtgcaagatacaaggctttagagagcagcataaatgttgacatgggacatttgctcatggaattggagctcgtgggacagtcacctcatggaattggagctcgtggaacagttacctctgcctcagaaaacaaggatgaattaagtttttttttaaaaaagaaacatttggtaaggggaattgaggacactgatatgggtcttgataaatggcttcctggcaatagtcaaattgtgtgaaaggtacttcaaatccttgaagatttaccacttgtgttttgcaagccagattttcctgaaaacccttgccatgtgctagtaattggaaaggcagctctaaatgtcaatcagcctagttgatcagcttattgtctagtgaaactcgttaatttgtagtgttggagaagaactgaaatcatacttcttagggttatgattaagtaatgataactggaaacttcagcggtttatataagcttgtattcctttttctctcctctccccatgatgtttagaaacacaactatattgtttgctaagcattccaactatctcatttccaagcaagtattagaataccacaggaaccacaagactgcacatcaaaatatgccccattcaacatctagtgagcagtcaggaaagagaacttccagatcctggaaatcagggttagtattgtccaggtctaccaaaaatctcaatatttcagataatcacaatacatcccttacctgggaaagggctgttataatctttcacaggggacaggatggttcccttgatgaagaagttgatatgccttttcccaactccagaaagtgacaagctcacagacctttgaactagagtttagctggaaaagtatgttagtgcaaattgtcacaggacagcccttctttccacagaagctccaggtagagggtgtgtaagtagataggccatgggcactgtgggtagacacacatgaagtccaagcatttagatgtataggttgatggtggtatgttttcaggctagatgtatgtacttcatgctgtctacactaagagagaatgagagacacactgaagaagcaccaatcatgaattagttttatatgcttctgttttataattttgtgaagcaaaattttttctctaggaaatatttattttaataatgtttcaaacatatattacaatgctgtattttaaaagaatgattatgaattacatttgtataaaataatttttatatttgaaatattgactttttatggcactagtatttttatgaaatattatgttaaaactgggacaggggagaacctagggtgatattaaccaggggccatgaatcaccttttggtctggagggaagccttggggctgatcgagttgttgcccacagctgtatgattcccagccagacacagcctcttagatgcagttctgaagaagatggtaccaccagtctgactgtttccatcaagggtacactgccttctcaactccaaactgactcttaagaagactgcattatatttattactgtaagaaaatatcacttgtcaataaaatccatacatttgtgt

SEQ ID NO: 35 - protein, 1480 aa, Homo sapiens
MQRSPLEKASVVSKLFFSWTRPILRKGYRQRLELSDIYQIPSVDSADNLSEKLEREWDRELASKKNPKLINALRRCFFWRFMFYGIFLYLGEVTKAVQPLLLGRIIASYDPDNKEERSIAIYLGIGLCLLFIVRTLLLHPAIFGLHHIGMQMRIAMFSLIYKKTLKLSSRVLDKISIGQLVSLLSNNLNKFDEGLALAHFVWIAPLQVALLMGLIWELLQASAFCGLGFLIVLALFQAGLGRMMMKYRDQRAGKISERLVITSEMIENIQSVKAYCWEEAMEKMIENLRQTELKLTRKAAYVRYFNSSAFFFSGFFVVFLSVLPYALIKGIILRKIFTTISFCIVLRMAVTRQFPWAVQTWYDSLGAINKIQDFLQKQEYKTLEYNLTTTEVVMENVTAFWEEGFGELFEKAKQNNNNRKTSNGDDSLFFSNFSLLGTPVLKDINFKIERGQLLAVAGSTGAGKTSLLMMIMGELEPSEGKIKHSGRISFCSQFSWIMPGTIKENIIFGVSYDEYRYRSVIKACQLEEDISKFAEKDNIVLGEGGITLSGGQRARISLARAVYKDADLYLLDSPFGYLDVLTEKEIFESCVCKLMANKTRILVTSKMEHLKKADKILILNEGSSYFYGTFSELQNLQPDFSSKLMGCDSFDQFSAERRNSILTETLHRFSLEGDAPVSWTETKKQSFKQTGEFGEKRKNSILNPINSIRKFSIVQKTPLQMNGIEEDSDEPLERRLSLVPDSEQGEAILPRISVISTGPTLQARRRQSVLNLMTHSVNQGQNIHRKTTASTRKVSLAPQANLTELDIYSRRLSQETGLEISEEINEEDLKECLFDDMESIPAVTTWNTYLRYITVHKSLIFVLIWCLVIFLAEVAASLVVLWLLGNTPLQDKGNSTHSRNNSYAVIITSTSSYYVFYIYVGVADTLLAMGFFRGLPLVHTLITVSKILHHKMLHSVLQAPMSTLNTLKAGGILNRFSKDIAILDDLLPLTIFDFIQLLLIVIGAIAVVAVLQPYIFVATVPVIVAFIMLRAYFLQTSQQLKQLESEGRSPIFTHLVTSLKGLWTLRAFGRQPYFETLFHKALNLHTANWFLYLSTLRWFQMRIEMIFVIFFIAVTFISILTTGEGEGRVGIILTLAMNIMSTLQWAVNSSIDVDSLMRSVSRVFKFIDMPTEGKPTKSTKPYKNGQLSKVMIIENSHVKKDDIWPSGGQMTVKDLTAKYTEGGNAILENISFSISPGQRVGLLGRTGSGKSTLLSAFLRLLNTEGEIQIDGVSWDSITLQQWRKAFGVIPQKVFIFSGTFRKNLDPYEQWSDQEIWKVADEVGLRSVIEQFPGKLDFVLVDGGCVLSHGHKQLMCLARSVLSKAKILLLDEPSAHLDPVTYQIIRRTLKQAFADCTVILCEHRIEAMLECQQFLVIEENKVRQYDSIQKLLNERSLFRQAISPSDRVKLFPHRNSSKCKSKPQIAALKEETEEEVQDTRL

SEQ ID NO: 36 - DNA, 3168 bp, Homo sapiens
  CDS (39)..(1613)
cacaagacctggaattgacaggactcccaactagtacaatgacagaagataaggtcactgggaccctggttttcactgtcatcactgctgtgctgggttccttccagtttggatatgacattggtgtgatcaatgcacctcaacaggtaataatatctcactatagacatgttttgggtgttccactggatgaccgaaaagctatcaacaactatgttatcaacagtacagatgaactgcccacaatctcatactcaatgaacccaaaaccaaccccttgggctgaggaagagactgtggcagctgctcaactaatcaccatgctctggtccctgtctgtatccagctttgcagttggtggaatgactgcatcattctttggtgggtggcttggggacacacttggaagaatcaaagccatgttagtagcaaacattctgtcattagttggagctctcttgatggggttttcaaaattgggaccatctcatatacttataattgctggaagaagcatatcaggactatattgtgggctaatttcaggcctggttcctatgtatatcggtgaaattgctccaaccgctctcaggggagcacttggcacttttcatcagctggccatcgtcacgggcattcttattagtcagattattggtcttgaatttatcttgggcaattatgatctgtggcacatcctgcttggcctgtctggtgtgcgagccatccttcagtctctgctactctttttctgtccagaaagccccagatacctttacatcaagttagatgaggaagtcaaagcaaaacaaagcttgaaaagactcagaggatatgatgatgtcaccaaagatattaatgaaatgagaaaagaaagagaagaagcatcgagtgagcagaaagtctctataattcagctcttcaccaattccagctaccgacagcctattctagtggcactgatgctgcatgtggctcagcaattttccggaatcaatggcattttttactactcaaccagcatttttcagacggctggtatcagcaaacctgtttatgcaaccattggagttggcgctgtaaacatggttttcactgctgtctctgtattccttgtggagaaggcagggcgacgttctctctttctaattggaatgagtgggatgtttgtttgtgccatcttcatgtcagtgggacttgtgctgctgaataagttctcttggatgagttatgtgagcatgatagccatcttcctctttgtcagcttctttgaaattgggccaggcccgatcccctggttcatggtggctgagtttttcagtcaaggaccacgtcctgctgctttagcaatagctgcattcagcaattggacctgcaatttcattgtagctctgtgtttccagtacattgcggacttctgtggaccttatgtgtttttcctctttgctggagtgctcctggcctttaccctgttcacattttttaaagttccagaaaccaaaggaaagtcttttgaggaaattgctgcagaattccaaaagaagagtggctcagcccacaggccaaaagctgctgtagaaatgaaattcctaggagctacagagactgtgtaaaaaaaaaaccctgctttttgacatgaacagaaacaataagggaaccgtctgtttttaaatgatgattccttgagcattttatatccacatctttaagtattgttttatttttatgtgctctcatcagaaatgtcatcaaatattaccaaaaaagtatttttttaagttagagaatatatttttgatggtaagactgtaattaagtaaaccaaaaaggctagtttattttgttacactaaagggcaggtggttctaatatttttagctctgttctttataacaaggttcttctaaaattgaagagatttcaacatatcatttttttaacacataactagaaacctgaggatgcaacaaatatttatatatttgaatatcattaaattggaattttcttacccatatatcttatgttaaaggagatatggctagtggcaataagttccatgttaaaatagacaactcttccatttattgcactcagcttttttcttgagtactagaatttgtattttgcttaaaattttacttttgttctgtattttcatgtggaatggattatagagtatactaaaaaatgtctatagagaaaaactttcatttttggtaggcttatcaaaatctttcagcactcagaaaagaaaaccattttagttcctttatttaatggccaaatggtttttgcaagatttaacactaaaaaggtttcacctgatcatatagcgtgggttatcagttaacattaacatctattataaaaccatgttgattcccttctggtacaatcctttgagttatagtttgctttgctttttaattgaggacagcctggttttcacatacactcaaacaatcatgagtcagacatttggtatattacctcaaattcctaataagtttgatcaaatctaatgtaagaaaatttgaagtaaaggattgatcactttgttaaaaatattttctgaattattatgtctcaaaataagttgaaaaggtagggtttgaggattcctgagtgtgggcttctgaaacttcataaatgttcagcttcagacttttatcaaaatccctatttaattttcctggaaagactgattgttttatggtgtgttcctaacataaaataatcgtctcctttgacatttccttctttgtcttagctgtatacagattctagccaaactattctatggccattactaacacgcattgtacactatctatctgcctttacctacataggcaaattggaaatacacagatgattaaacagactttagcttacagtcaattttacaattatggaaatatagttctgatgggtcccaaaagcttagcagggtgctaacgtatctctaggctgttttctccaccaactggagcactgatcaatccttcttatgtttgctttaatgtgtattgaagaaaagcactttttaaaaagtactctttaagagtgaaataattaaaaaccactgaacatttgctttgttttctaaagttgttcacatatatgtaatttagcagtccaaagaacaagaaattgtttcttttc

SEQ ID NO: 37 - protein, 524 aa, Homo sapiens
MTEDKVTGTLVFTVITAVLGSFQFGYDIGVINAPQQVIISHYRHVLGVPLDDRKAINNYVINSTDELPTISYSMNPKPTPWAEEETVAAAQLITMLWSLSVSSFAVGGMTASFFGGWLGDTLGRIKAMLVANILSLVGALLMGFSKLGPSHILIIAGRSISGLYCGLISGLVPMYIGEIAPTALRGALGTFHQLAIVTGILISQIIGLEFILGNYDLWHILLGLSGVRAILQSLLLFFCPESPRYLYIKLDEEVKAKQSLKRLRGYDDVTKDINEMRKEREEASSEQKVSIIQLFTNSSYRQPILVALMLHVAQQFSGINGIFYYSTSIFQTAGISKPVYATIGVGAVNMVFTAVSVFLVEKAGRRSLFLIGMSGMFVCAIFMSVGLVLLNKFSWMSYVSMIAIFLFVSFFEIGPGPIPWFMVAEFFSQGPRPAALAIAAFSNWTCNFIVALCFQYIADFCGPYVFFLFAGVLLAFTLFTFFKVPETKGKSFEEIAAEFQKKSGSAHRPKAAVEMKFLGATETV

SEQ ID NO: 38 - DNA, 3915 bp, Homo sapiens
  CDS (243)..(1733)
gtggggtggggtggggctgggggcttgtcgccctttcaggctccaccctttgcggagattataaatagtcatgatcccagcgagacccagagatgcctgtaatggtgagactttggatccttcctgaggacgtggagaaaactttctgctgagaaggacattttgaaggttttgttggctgaaaaagctgtttctggaatcacccctagatctttcttgaagacttgaattagattacagcgatggggacacagaaggtcaccccagctctgatatttgccatcacagttgctacaatcggctctttccaatttggctacaacactggggtcatcaatgctcctgagaagatcataaaggaatttatcaataaaactttgacggacaagggaaatgccccaccctctgaggtgctgctcacgtctctctggtccttgtctgtggccatattttccgtcgggggtatgatcggctccttttccgtcggactcttcgtcaaccgctttggcaggcgcaattcaatgctgattgtcaacctgttggctgtcactggtggctgctttatgggactgtgtaaagtagctaagtcggttgaaatgctgatcctgggtcgcttggttattggcctcttctgcggactctgcacaggttttgtgcccatgtacattggagagatctcgcctactgccctgcggggtgcctttggcactctcaaccagctgggcatcgttgttggaattctggtggcccagatctttggtctggaattcatccttgggtctgaagagctatggccgctgctactgggttttaccatccttcctgctatcctacaaagtgcagcccttccattttgccctgaaagtcccagatttttgctcattaacagaaaagaagaggagaatgctaagcagatcctccagcggttgtggggcacccaggatgtatcccaagacatccaggagatgaaagatgagagtgcaaggatgtcacaagaaaagcaagtcaccgtgctagagctctttagagtgtccagctaccgacagcccatcatcatttccattgtgctccagctctctcagcagctctctgggatcaatgctgtgttctattactcaacaggaatcttcaaggatgcaggtgttcaagagcccatctatgccaccatcggcgcgggtgtggttaatactatcttcactgtagtttctctatttctggtggaaagggcaggaagaaggactctgcatatgataggccttggagggatggctttttgttccacgctcatgactgtttctttgttattaaaggataactataatgggatgagctttgtctgtattggggctatcttggtctttgtagccttctttgaaattggaccaggccccattccctggtttattgtggccgaactcttcagccagggcccccgcccagctgcgatggcagtggccggctgctccaactggacctccaacttcctagtcggattgctcttcccctccgctgctcactatttaggagcctacgtttttattatcttcaccggcttcctcattaccttcttggcttttaccttcttcaaagtccctgagacccgtggcaggacttttgaggatatcacacgggcctttgaagggcaggcacacggtgcagatagatctggaaaggacggcgtcatggagatgaacagcatcgagcctgctaaggagaccaccaccaatgtctaagtcgtgcctccttccacctccctcccggcatgggaaagccacctctccctcaacaagggagagacctcatcaggatgaacccaggacgcttctgaatgctgctacttaattcctttctcatcccacgcactccatgagcaccccaaggctgcggtttgttggatcttcaatggctttttaaattttatttcctggacatcctcttctgcttaggagagaccgagtgaacctaccttcatttcaggagggattggccgcttggcacatgacaactttgccagcttttcctcccttgggttctgatattgccgcactaggggatataggagaggaaaagtaaggtgcagttcccccaacctcagacttaccaggaagcagatacatatgagtgtggaagccggagggtgtttatgtaagagcaccttcctcacttccatacagctctacgtggcaaattaacttgagttttatttattttatcctctggtttaattacataatttttttttttttactttaagtttcaggatacatgtgccgaatgtgcaggtttgttacataggtatatatatgccatgatggaaatatttatttttttaagcgtaattttgccaaataataaaaacagaaggaaattgagattagagggaggtgtttaaagagaggttatagagtagaagatttgatgctggagaggttaaggtgcaataagaatttagggagaaatgttgttcattattggagggtaaatgatgtggtgcctgaggtctgtacgttacctcttaacaatttctgtccttcagatggaaactctttaacttctcgtaaaagtcatatacctatataataaagctactgatttccttggagcttttttctttaagataatagtttacatgtagtagtacttgaaatctaggattattaactaatatgggcattgtagttaatgatggttgatgggttctaattttggatggagtccagggaagagaaagtgatttctagaaagcctgttcccctcactggatgaaataactccttcttgtagtagtctcattacttttgaagtaatcccgccacctatctcgtgggagagccatccaaataagaaacctaaaataattggttcttggtagagattcattatttttccactttgttctttaggagattttaggtgttgattttctgttgtattttaactcatacctttaaaggaattccccaaagaatgtttatagcaaacttggaatttgtaacctcagctctgggagaggatttttttctgagcgattattatctaaagtgtgttgttgctttaggctcacggcacgcttgcgtatgtctgttaccatgtcactgtggtcctatgccgaatgccctcaggggacttgaatctttccaataaaccaggtttagacagtatgagtcaatgtgcagtgtagcccacacttgagaggatgaatgtatgtgcactgtcactttgctctgggtggaagtacgttattgttgacttattttctctgtgtttgttcctacagcccctttttcatatgttgctcagtctccctttcccttcttggtgcttacacatctcagaccctttagccaaacccttgtcagtgacagtattttggttcttagttctcactgttccctctgctcctggagcctttgaataaaaatgcacgtagctgaggccggatgcggtggctcacgcctgtaatcccagcactttgggaggcctaggcgggcggtcaggggttcgagaccagtctggccaacatcgtgaaaccctgtctctactaaaaatgcaaaaattagccgggcgtggtggcgggcgcctgtaatcccagctacttgggaagctgaggcgggagaatcatgtgaacccgggacgcaggggttgcagtgagcggagatcgcatcattgcactctagcctgggccacagggcgagactccgtctcaaaaaaaaaaaaatgcacatagctatcgagtgtgctttagcttgaaaaggtgaccttgcaacttcatgtcaactttctggctcctcaaacagtaggttggcagtaaggcagggtcccatttctcactgagaagattgtgaatatttccatatggattttctattgttactctggttctttgttttaaaataaaaattctgaatgtacacg

SEQ ID NO: 39 - protein, 496 aa, Homo sapiens
MGTQKVTPALIFAITVATIGSFQFGYNTGVINAPEKIIKEFINKTLTDKGNAPPSEVLLTSLWSLSVAIFSVGGMIGSFSVGLFVNRFGRRNSMLIVNLLAVTGGCFMGLCKVAKSVEMLILGRLVIGLFCGLCTGFVPMYIGEISPTALRGAFGTLNQLGIVVGILVAQIFGLEFILGSEELWPLLLGFTILPAILQSAALPFCPESPRFLLINRKEEENAKQILQRLWGTQDVSQDIQEMKDESARMSQEKQVTVLELFRVSSYRQPIIISIVLQLSQQLSGINAVFYYSTGIFKDAGVQEPIYATIGAGVVNTIFTVVSLFLVERAGRRTLHMIGLGGMAFCSTLMTVSLLLKDNYNGMSFVCIGAILVFVAFFEIGPGPIPWFIVAELFSQGPRPAAMAVAGCSNWTSNFLVGLLFPSAAHYLGAYVFIIFTGFLITFLAFTFFKVPETRGRTFEDITRAFEGQAHGADRSGKDGVMEMNSIEPAKETTTNV

SEQ ID NO: 40 - DNA, 2218 bp, Homo sapiens
  CDS (1)..(1581)
cttctctctccattcagtgcacgcgttactttggctaaaaggaggtgagcggcactctgcccttccagagcaagcatggagcaacaggatcagagcatgaaggaagggaggctgacgcttgtgcttgccctggcaaccctgatagctgcctttgggtcatccttccagtatgggtacaacgtggctgctgtcaactccccagcactgctcatgcaacaattttacaatgagacttactatggtaggaccggtgaattcatggaagacttccccttgacgttgctgtggtctgtaaccgtgtccatgtttccatttggagggtttatcggatccctcctggtcggccccttggtgaataaatttggcagaaaaggggccttgctgttcaacaacatattttctatcgtgcctgcgatcttaatgggatgcagcagagtcgccacatcatttgagcttatcattatttccagacttttggtgggaatatgtgcaggtgtatcttccaacgtggtccccatgtacttaggggagctggcccctaaaaacctgcggggggctctcggggtggtgccccagctcttcatcactgttggcatccttgtggcccagatctttggtcttcggaatctccttgcaaacgtagatggctggccgatcctgctggggctgaccggggtccccgcggcgctgcagctccttctgctgcccttcttccccgagagccccaggtacctgctgattcagaagaaagacgaagcggccgccaagaaagccctacagacgctgcgcggctgggactctgtggacagggaggtggccgagatccggcaggaggatgaggcagagaaggccgcgggcttcatctccgtgctgaagctgttccggatgcgctcgctgcgctggcagctgctgtccatcatcgtcctcatgggcggccagcagctgtcgggcgtcaacgctatctactactacgcggaccagatctacctgagcgccggcgtgccggaggagcacgtgcagtacgtgacggccggcaccggggccgtgaacgtggtcatgaccttctgcgccgtgttcgtggtggagctcctgggtcggaggctgctgctgctgctgggcttctccatctgcctcatagcctgctgcgtgctcactgcagctctggcactgcaggacacagtgtcctggatgccatacatcagcatcgtctgtgtcatctcctacgtcataggacatgccctcgggcccagtcccatacccgcgctgctcatcactgagatcttcctgcagtcctctcggccatctgccttcatggtggggggcagtgtgcactggctctccaacttcaccgtgggcttgatcttcccgttcatccaggagggcctcggcccgtacagcttcattgtcttcgccgtgatctgcctcctcaccaccatctacatcttcttgattgtcccggagaccaaggccaagacgttcatagagatcaaccagattttcaccaagatgaataaggtgtctgaagtgtacccggaaaaggaggaactgaaagagcttccacctgtcacttcggaacagtgactctggagaggaagccagtggagctggtctgccaggggcttcccactttggcttatttttctgacttctagctgtctgtgaatatccagaaataaaacaactctgatgtggaatgcagtcctcatctccagcctccccaccccagtgggaactgtgcaaagggctgccttgctgttcttgaagctgggctgtctctctccatgttggcctgtcaccagacccgagtcaattaaacagctggtcctccactttgctggttcagccttcgtgtggctcctggtaacgtggctccaccttgatgggtcaacctttgtgtggctcctggtaacataacaacaacagttactatagtggtgagatggaaggaatcaaattttgccagagaaactaactcggtggccccaacaggtcttccggggccatgggcatttgtttagagccaaattcatcctcttaccagatccttttccagaaatacctgtctaggaaggtgtgatgtcagaaacaatgacatccagaaagctgaggaacaggttcctgtggagacactgagtcagaattcttcatccaaattattttgttagtggaaaatggaattgcttctgtgtagtcaataaaatgaacctgatcacttttc

SEQ ID NO: 41 - protein, 15 aa, Homo sapiens
LLSPFSARVTLAKRR

SEQ ID NO: 42 - protein, 510 aa, Homo sapiens
AALCPSRASMEQQDQSMKEGRLTLVLALATLIAAFGSSFQYGYNVAAVNSPALLMQQFYNETYYGRTGEFMEDFPLTLLWSVTVSMFPFGGFIGSLLVGPLVNKFGRKGALLFNNIFSIVPAILMGCSRVATSFELIIISRLLVGICAGVSSNVVPMYLGELAPKNLRGALGVVPQLFITVGILVAQIFGLRNLLANVDGWPILLGLTGVPAALQLLLLPFFPESPRYLLIQKKDEAAAKKALQTLRGWDSVDREVAEIRQEDEAEKAAGFISVLKLFRMRSLRWQLLSIIVLMGGQQLSGVNAIYYYADQIYLSAGVPEEHVQYVTAGTGAVNVVMTFCAVFVVELLGRRLLLLLGFSICLIACCVLTAALALQDTVSWMPYISIVCVISYVIGHALGPSPIPALLITEIFLQSSRPSAFMVGGSVHWLSNFTVGLIFPFIQEGLGPYSFIVFAVICLLTTIYIFLIVPETKAKTFIEINQIFTKMNKVSEVYPEKEELKELPPVTSEQ

SEQ ID NO: 43 - DNA, 1580 bp, Homo sapiens
  CDS (47)..(1570)
tgctccagtctgagcgccctccgctcgccccgagagagacccggccatgcaggagccgctgctgggagccgagggcccggactacgacaccttccccgagaagccgcccccgtcgccaggggacagggcgcgggtcgggaccctgcagaacaaaagggtgttcctggccaccttcgccgcagtgctcggcaatttcagctttgggtatgccctggtctacacatcccctgtcatcccagccctggagcgctccttggatcctgacctgcatctgaccaaatcccaggcatcctggtttgggtccgtgttcaccctgggagcagcggccggaggcctgagtgccatgatcctcaacgacctcctgggccggaagctgagcatcatgttctcagctgtgccgtcggcggccggctatgcgctcatggcgggtgcgcacggcctctggatgctgctgctcggaaggacgctgacgggcttcgccggggggctcacagctgcctgcatcccggtgtacgtgtctgagattgctcccccaggcgttcgtggggctctgggggccacaccccagctcatggcagtgttcggatccctgtccctctacgcccttggcctcctgctgccgtggcgctggctggctgtggccggggaggcgcctgtgctcatcatgatcctgctgctcagcttcatgcccaactcgccgcgcttcctgctctctcggggcagggacgaagaggccctgcgggcgctggcctggctgcgtgggacggacgtcgatgtccactgggagttcgagcagatccaggacaacgtccggagacagagcagccgagtatcgtgggctgaggcacgggccccacacgtgtgccggcccatcaccgtggccttgctgatgcgcctcctgcagcagctgacgggcatcacgcccatcctggtctacctgcagtccatcttcgacagcaccgctgtcctgctgccccccaaggacgacgcagccatcgttggggccgtgcggctcctgtccgtgctgatcgccgccctcaccatggacctcgcaggccgcaaggtgctgctcttcgtctcagcggccatcatgtttgctgccaacctgactctggggctgtacatccactttggccccaggcctctgagccccaacagcactgcgggcctggaaagcgagtcctggggggacttggcgcagcccctggcagcacccgctggctacctcaccctggtgcccctgctggccaccatgctcttcatcatgggctacgccgtgggctggggtcccatcacctggctgctcatgtctgaggtcctgcccctgcgtgcccgtggcgtggcctcagggctctgcgtgctggccagctggctcaccgccttcgtcctcaccaagtccttcctgccagtggtgagcaccttcggcctccaggtgcctttcttcttcttcgcggccatctgcttggtgagcctggtgttcacaggctgctgtgtgcccgagaccaagggacggtccctggagcagatcgagtccttcttccgcatggggagaaggtccttcttgcgctaggtcaaggtcc

SEQ ID NO: 44 - protein, 507 aa, Homo sapiens
MQEPLLGAEGPDYDTFPEKPPPSPGDRARVGTLQNKRVFLATFAAVLGNFSFGYALVYTSPVIPALERSLDPDLHLTKSQASWFGSVFTLGAAAGGLSAMILNDLLGRKLSIMFSAVPSAAGYALMAGAHGLWMLLLGRTLTGFAGGLTAACIPVYVSEIAPPGVRGALGATPQLMAVFGSLSLYALGLLLPWRWLAVAGEAPVLIMILLLSFMPNSPRFLLSRGRDEEALRALAWLRGTDVDVHWEFEQIQDNVRRQSSRVSWAEARAPHVCRPITVALLMRLLQQLTGITPILVYLQSIFDSTAVLLPPKDDAAIVGAVRLLSVLIAALTMDLAGRKVLLFVSAAIMFAANLTLGLYIHFGPRPLSPNSTAGLESESWGDLAQPLAAPAGYLTLVPLLATMLFIMGYAVGWGPITWLLMSEVLPLRARGVASGLCVLASWLTAFVLTKSFLPVVSTFGLQVPFFFFAAICLVSLVFTGCCVPETKGRSLEQIESFFRMGRRSFLR

SEQ ID NO: 45 - DNA, 1575 bp, Homo sapiens
  CDS (1)..(1575)
atggagaacaaagaggcgggaacccctccacccattccatccagggaggggcggctccagccgacgctgttgctggcgacactgagcgcggcctttggctcagccttccagtacggctacaacctctctgtggtcaacacgccgcacaaggtgggcacaagctgtggatggggcaatgttttccaggtcttcaagtcattttacaacgaaacctactttgagcgacacgcaacattcatggacgggaagctcatgctgcttctatggtcttgcaccgtctccatgtttcctctgggcggcctgttggggtcattgctcgtgggcctgctggttgatagctgcggcagaaaggggaccctgctgatcaacaacatctttgccatcatccccgccatcctgatgggagtcagcaaagtggccaaggcttttgagctgatcgtcttttcccgagtggtgctgggagtctgtgcaggcatctcctacagcgcccttcccatgtacctgggagaactggcccccaagaacctgagaggcatggtgggaacaatgaccgaggttttcgtcatcgttggagtcttcctagcacagatcttcagcctccaggccatcttgggcaacccggcaggctggccggtgcttctggcgctcacaggggtgcccgccctgctgcagctgctgaccctgcccttcttccccgaaagcccccgctactccctgattcagaaaggagatgaagccacagcgcgacaagctctgaggaggctgagaggccacacggacatggaggccgagctggaggacatgcgtgcggaggcccgggccgagcgcgccgagggccacctgtctgtgctgcacctctgtgccctgcggtccctgcgctggcagctcctctccatcatcgtgctcatggccggccagcagctgtcgggcatcaatgcgatcaactactatgcggacaccatctacacatctgcgggcgtggaggccgctcactcccaatatgtaacggtgggctctggcgtcgtcaacatagtgatgaccatcacctcggctgtccttgtggagcggctgggacggcggcacctcctgctggccggctacggcatctgcggctctgcctgcctggtgctgacggtggtgctcctattccagaacagggtccccgagctgtcctacctcggcatcatctgtgtctttgcctacatcgcgggacattccattgggcccagtcctgtcccctcggtggtgaggaccgagatcttcctgcagtcctcccggcgggcagctttcatggtggacggggcagtgcactggctcaccaacttcatcataggcttcctgttcccatccatccaggaggccatcggtgcctacagtttcatcatctttgccggaatctgcctcctcactgcgatttacatctacgtggttattccggagaccaagggcaaaacatttgtggagataaaccgcatttttgccaagagaaacagggtgaagcttccagaggagaaagaagaaaccattgatgctgggcctcccacagcctctcctgccaaggaaacttccttttag

SEQ ID NO: 46 - protein, 524 aa, Homo sapiens
MENKEAGTPPPIPSREGRLQPTLLLATLSAAFGSAFQYGYNLSVVNTPHKVGTSCGWGNVFQVFKSFYNETYFERHATFMDGKLMLLLWSCTVSMFPLGGLLGSLLVGLLVDSCGRKGTLLINNIFAIIPAILMGVSKVAKAFELIVFSRVVLGVCAGISYSALPMYLGELAPKNLRGMVGTMTEVFVIVGVFLAQIFSLQAILGNPAGWPVLLALTGVPALLQLLTLPFFPESPRYSLIQKGDEATARQALRRLRGHTDMEAELEDMRAEARAERAEGHLSVLHLCALRSLRWQLLSIIVLMAGQQLSGINAINYYADTIYTSAGVEAAHSQYVTVGSGVVNIVMTITSAVLVERLGRRHLLLAGYGICGSACLVLTVVLLFQNRVPELSYLGIICVFAYIAGHSIGPSPVPSVVRTEIFLQSSRRAAFMVDGAVHWLTNFIIGFLFPSIQEAIGAYSFIIFAGICLLTAIYIYVVIPETKGKTFVEINRIFAKRNRVKLPEEKEETIDAGPPTASPAKETSF

SEQ ID NO: 47 - DNA, 1508 bp, Homo sapiens
  CDS (27)..(1460)
agctggccgatgcgttggccgccgacatgacgcccgaggacccagaggaaacccagccgcttctggggcctcctggcggcagcgcgccccgcggccgccgcgtcttcctcgccgccttcgccgctgccctgggcccactcagcttcggcttcgcgctcggctacagctccccggccatccctagcctgcagcgcgccgcgcccccggccccgcgcctggacgacgccgccgcctcctggttcggggctgtcgtgaccctgggtgccgcggcggggggagtgctgggcggctggctggtggaccgcgccgggcgcaagctgagcctcttgctgtgctccgtgcccttcgtggccggctttgccgtcatcaccgcggcccaggacgtgtggatgctgctggggggccgcctcctcaccggcctggcctgcggtgttgcctccctagtggccccggtctacatctccgaaatcgcctacccagcagtccgggggttgctcggctcctgtgtgcagctaatggtcgtcgtcggcatcctcctggcctacctggcaggctgggtgctggagtggcgctggctggctgtgctgggctgcgtgcccccctccctcatgctgcttctcatgtgcttcatgcccgagaccccgcgcttcctgctgactcagcacaggcgccaggaggccatggccgccctgcggttcctgtggggctccgagcagggctgggaagacccccccatcggggctgagcagagctttcacctggccctgctgcggcagcccggcatctacaagcccttcatcatcggcgtctccctgatggccttccagcagctgtcgggggtcaacgccgtcatgttctatgcagagaccatctttgaagaggccaagttcaaggacagcagcctggcctcggtcgtcgtgggtgtcatccaggtgctgttcacagctgtggcggctctcatcatggacagagcagggcggaggctgctcctggtcttgtcaggtgtggtcatggtgttcagcacgagtgccttcggcgcctacttcaagctgacccagggtggccctggcaactcctcgcacgtggccatctcggcgcctgtctctgcacagcctgttgatgccagcgtggggctggcctggctggccgtgggcagcatgtgcctcttcatcgccggctttgcggtgggctgggggcccatcccctggctcctcatgtcagagatcttccctctgcatgtcaagggcgtggcgacaggcatctgcgtcctcaccaactggctcatggcctttctcgtgaccaaggagttcagcagcctcatggaggtcctcaggccctatggagccttctggcttgcctccgctttctgcatcttcagtgtccttttcactttttcctgtgtccctgaaactaaaggaaagactctggaacaaatcacagcccattttgaggggcgatgacagccactcactaggggatggagcaagcctgtgactccaagctgggcc

SEQ ID NO: 48 - protein, 477 aa, Homo sapiens [sequence omitted]

SEQ ID NO: 49 - DNA, 1863 bp, Homo sapiens
  CDS (55)..(1677)
cttggcagagtctggggtccctggactgagccatcagctgggtcactgagacccatggcaaggaaacaaaataggaattccaaggaactgggcctagttcccctcacagatgacaccagccacgccaggcctccagggccagggagggcactgctggagtgtgaccacctgaggagtggggtgccaggtggaaggagaagaaaggactggtcctgctcgctcctcgtggcctccctcgcgggcgccttcggctcctccttcctctacggctacaacctgtcggtggtgaatgcccccaccccgtacatcaaggccttttacaatgagtcatgggaaagaaggcatggacgtccaatagacccagacactctgactctgctctggtctgtgactgtgtccatattcgccatcggtggacttgtggggacattaattgtgaagatgattggaaaggttcttgggaggaagcacactttgctggccaataatgggtttgcaatttctgctgcattgctgatggcctgctcgctccaggcaggagcctttgaaatgctcatcgtgggacgcttcatcatgggcatagatggaggcgtcgccctcagtgtgctccccatgtacctcagtgagatctcacccaaggagatccgtggctctctggggcaggtgactgccatctttatctgcattggcgtgttcactgggcagcttctgggcctgcccgagctgctgggaaaggagagtacctggccatacctgtttggagtgattgtggtccctgccgttgtccagctgctgagccttccctttctcccggacagcccacgctacctgctcttggagaagcacaacgaggcaagagctgtgaaagccttccaaacgttcttgggtaaagcagacgtttcccaagaggtagaggaggtcctggctgagagccgcgtgcagaggagcatccgcctggtgtccgtgctggagctgctgagagctccctacgtccgctggcaggtggtcaccgtgattgtcaccatggcctgctaccagctctgtggcctcaatgcaatttggttctataccaacagcatctttggaaaagctgggatccctctggcaaagatcccatacgtcaccttgagtacagggggcatcgagactttggctgccgtcttctctggtttggtcattgagcacctgggacggagacccctcctcattggtggctttgggctcatgggcctcttctttgggaccctcaccatcacgctgaccctgcaggaccacgccccctgggtcccctacctgagtatcgtgggcattctggccatcatcgcctctttctgcagtgggccaggtggcatcccgttcatcttgactggtgagttcttccagcaatctcagcggccggctgccttcatcattgcaggcaccgtcaactggctctccaactttgctgttgggctcctcttcccattcattcagaaaagtctggacacctactgtttcctagtctttgctacaatttgtatcacaggtgctatctacctgtattttgtgctgcctgagaccaaaaacagaacctatgcagaaatcagccaggcattttccaaaaggaacaaagcatacccaccagaagagaaaatcgactcagctgtcactgatggtaagataaatggaaggccttaacaagtttcctcctccacgttggacaattatgtcaaaaacaggattgtctacatggatgatctcacttttcaggaaacttaaaatttacccattattgggaagcttaaatgaattgaagctatgcaagtcttttatattattaaatatttaaaagtaaacctgtactaatctaaaaaaaaaaaaaaa

SEQ ID NO: 50 - protein, 540 aa, Homo sapiens
MARKQNRNSKELGLVPLTDDTSHARPPGPGRALLECDHLRSGVPGGRRRKDWSCSLLVASLAGAFGSSFLYGYNLSVVNAPTPYIKAFYNESWERRHGRPIDPDTLTLLWSVTVSIFAIGGLVGTLIVKMIGKVLGRKHTLLANNGFAISAALLMACSLQAGAFEMLIVGRFIMGIDGGVALSVLPMYLSEISPKEIRGSLGQVTAIFICIGVFTGQLLGLPELLGKESTWPYLFGVIVVPAVVQLLSLPFLPDSPRYLLLEKHNEARAVKAFQTFLGKADVSQEVEEVLAESRVQRSIRLVSVLELLRAPYVRWQVVTVIVTMACYQLCGLNAIWFYTNSIFGKAGIPLAKIPYVTLSTGGIETLAAVFSGLVIEHLGRRPLLIGGFGLMGLFFGTLTITLTLQDHAPWVPYLSIVGILAIIASFCSGPGGIPFILTGEFFQQSQRPAAFIIAGTVNWLSNFAVGLLFPFIQKSLDTYCFLVFATICITGAIYLYFVLPETKNRTYAEISQAFSKRNKAYPPEEKIDSAVTDGKINGRP

SEQ ID NO: 51 - DNA, 4167 bp, Homo sapiens
  CDS (53)..(1678)
atgcgcgcccggcccctcagcgcccccagcacgccgccgagtcccgctcgccatgggccactccccacctgtcctgcctttgtgtgcctctgtgtctttgctgggtggcctgacctttggttatgaactggcagtcatatcaggtgccctgctgccactgcagcttgactttgggctaagctgcttggagcaggagttcctggtgggcagcctgctcctgggggctctcctcgcctccctggttggtggcttcctcattgactgctatggcaggaagcaagccatcctcgggagcaacttggtgctgctggcaggcagcctgaccctgggcctggctggttccctggcctggctggtcctgggccgcgctgtggttggcttcgccatttccctctcctccatggcttgctgtatctacgtgtcagagctggtggggccacggcagcggggagtgctggtgtccctctatgaggcaggcatcaccgtgggcatcctgctctcctatgccctcaactatgcactggctggtaccccctggggatggaggcacatgttcggctgggccactgcacctgctgtcctgcaatccctcagcctcctcttcctccctgctggtacagatgagactgcaacacacaaggacctcatcccactccagggaggtgaggcccccaagctgggcccggggaggccacggtactcctttctggacctcttcagggcacgcgataacatgcgaggccggaccacagtgggcctggggctggtgctcttccagcaactaacagggcagcccaacgtgctgtgctatgcctccaccatcttcagctccgttggtttccatgggggatcctcagccgtgctggcctctgtggggcttggcgcagtgaaggtggcagctaccctgaccgccatggggctggtggaccgtgcaggccgcagggctctgttgctagctggctgtgccctcatggccctgtccgtcagtggcataggcctcgtcagctttgccgtgcccatggactcaggcccaagctgtctggctgtgcccaatgccaccgggcagacaggcctccctggagactctggcctgctgcaggactcctctctacctcccattccaaggaccaatgaggaccaaagggagccaatcttgtccactgctaagaaaaccaagccccatcccagatctggagacccctcagcccctcctcggctggccctgagctctgccctccctgggccccctctgcccgctcgggggcatgcactgctgcgctggaccgcactgctgtgcctgatggtctttgtcagtgccttctcctttgggtttgggccagtgacctggcttgtcctcagcgagatctaccctgtggagatacgaggaagagccttcgccttctgcaacagcttcaactgggcggccaacctcttcatcagcctctccttcctcgatctcattggcaccatcggcttgtcctggaccttcctgctctacggactgaccgctgtcctcggcctgggcttcatctatttatttgttcctgaaacaaaaggccagtcgttggcagagatagaccagcagttccagaagagacggttcaccctgagctttggccacaggcagaactccactggcatcccgtacagccgcatcgagatctctgcggcctcctgaggaatccgtctgcctggaaattctggaactgtggctttggcagaccatctccagcatcctgcttcctaggccccagagcacaagttccagctggtcttttgggagtggcccctgcccccaaaggtggtctgcttttgctggggtaaaaaggatgaaagtctgagaatgcccaactcttcattttgagtctcaggccctgaaggttcctgaggatctagcttcatgcctcagtttccccattgacttgcacatctctgcagtatttataagaagaatattctatgaagtctttgttgcaccatggacttttctcaaagaatctcaagggtaccaatcctggcaggaagtctctcccgatatcacccctaaatccaaatgaggatatcatcttttctaatctcttttttcaactggctgggacattttcggaagggggaagtctctttttttactcttatcattttttttttttgaggtggagtctcattctgttgcccaggctggcctgatcttggctcactgcaacctccacctcctgagttcaagcgattcttgtgcctcagcctcctaagcagctgggactacaggcgcatgcaaccatacccagctaatttatttttagcagagatggggtttcactgtgttggccaggctggtcgtgaactcctgagctcaagtgatccacccacctcagcctcccagagtgctaggattacaggccttttgactcttttatctgagttttattgacccctctaattctcttacccagaatatttatccttcaccagcaactctgactctttgacgggaggcctcagttctagtccttggtctgctggtgtcattgctgtaggaatgaccacgggcctcagtttccccatttgtataatgggaagcctgtaccaggtcattcttaagatttctcctgactccagtgagctggaattctaaatgctggtctaggagctgtctccaggatggtgcaggatggctttgcggaaaggagatgggtttggaggccaacaaacctgcttgtcaatattgcctttgcctcttggcagcccttgaacttgagtaaataacaactccctgaacctcagtttcctcatctgcagaatggggataattatgtcccaggggtatatttagaccctgtttcctttcaggagggtccccagctggtccagggcctgggaaatttctacttatcctcattacccaggtccctcctttggaccctgtaaagggtcagggtgaatcagatgggggactgagcaagtagctatgactgcagatcatgtaaggaagggactgacaagaagctcccagatgctggggagaatgaagagctaaaatagatcctaggtgctggatgctttgtcatccatgcgtgcacatatgggtgctggcagagcccccaaggactctggcctctcgagttctcctatcttctccattctagatgcttcccttgtatccagtgatgtgctggagctggctttgccaagcttgtgagagctggttgctacattttcaggatttttacaagttggtaaacacagccattataaaaaattaaatgatttaaatttataattaagtaaattacattaaaacaaaaaaattatactcaaaattcattacttaattttactacctgttactattatctgtgcttttgaggctatttctacatagtaactcttatggagacctaggggagacaccgcgcatctcttcctgattccccactcaatgacatcatgttagtctttggttgcttaactggctgtggggagtgtttttgtatcacaaagattagagaggactacacatcagggcttgatttattgtttgttgattttctagacttcagaacatgctggataaaatgtcagtaatgcaaattaaactttaaagtatgtcttgtttgtagccaatacatggtgtatagcaccaaaaaatggagggattattcttccagtagttgaacactgtcatccgtttcagctgacagctgctcaaatcatttaagaaggagttctgacattcattttcattgttttacttttgtcttcctcactagtgtaaacaaaaatttcaaccagcattcatgccgaacctatacccattcttcagtgcctagctgtacagttatcagggatttttatttgtagtctaattttgtcaaatcatggccaaatcgcagtgatagttgactttggatacaaggtttggcaaaaaaaaaaatattaacaaaatattctgtaagaatcaattgtctatatggaatttaggataaagaatatttacaataaagaatatttacaataaagagtttattattatttgtaagttgtgtgcaacaaacataccctttatctctgtaaaatttatacacacaaaaattaacaaaagattctgtaagaattaattggctatatggaatttaggatagaatatttacaataaagagtatttacaat

SEQ ID NO: 52 - protein, 541 aa, Homo sapiens
MGHSPPVLPLCASVSLLGGLTFGYELAVISGALLPLQLDFGLSCLEQEFLVGSLLLGALLASLVGGFLIDCYGRKQAILGSNLVLLAGSLTLGLAGSLAWLVLGRAVVGFAISLSSMACCIYVSELVGPRQRGVLVSLYEAGITVGILLSYALNYALAGTPWGWRHMFGWATAPAVLQSLSLLFLPAGTDETATHKDLIPLQGGEAPKLGPGRPRYSFLDLFRARDNMRGRTTVGLGLVLFQQLTGQPNVLCYASTIFSSVGFHGGSSAVLASVGLGAVKVAATLTAMGLVDRAGRRALLLAGCALMALSVSGIGLVSFAVPMDSGPSCLAVPNATGQTGLPGDSGLLQDSSLPPIPRTNEDQREPILSTAKKTKPHPRSGDPSAPPRLALSSALPGPPLPARGHALLRWTALLCLMVFVSAFSFGFGPVTWLVLSEIYPVEIRGRAFAFCNSFNWAANLFISLSFLDLIGTIGLSWTFLLYGLTAVLGLGFIYLFVPETKGQSLAEIDQQFQKRRFTLSFGHRQNSTGIPYSRIEISAAS

SEQ ID NO: 53 - DNA, 1608 bp, Homo sapiens
  CDS (100)..(1590)
cggacctgcctctcacgcaatggatcccctggcggcaacccgagaccggttctcctacccgcattccgccaagtctctcgctctgcccaggacgcacagatgagagcgctccgaagactgattcagggcaggatcctgctcctgaccatctgcgctgccggcattggtgggacttttcagtttggctataacctctctatcatcaatgccccgaccttgcacattcaggaattcaccaatgagacatggcaggcgcgtactggagagccactgcccgatcacctagtcctgcttatgtggtccctcatcgtgtctctgtatcccctgggaggcctctttggagcactgcttgcaggtcccttggccatcacgctgggaaggaagaagtccctcctggtgaataacatctttgtggtgtcagcagcaatcctgtttggattcagccgcaaagcaggctcctttgagatgatcatgctgggaagactgctcgtgggagtcaatgcaggtgtgagcatgaacatccagcccatgtacctgggggagagcgcccctaaggagctccgaggagctgtggccatgagctcagccatctttacggctctggggatcgtgatgggacaggtggtcggactcagggagctcctaggtggccctcaggcctggcccctgctgctggccagctgcctggtgcccggggcgctccagctcgcctccctgcctctgctccctgaaagcccgcgctacctcctcattgactgtggagacaccgaggcctgcctggcagcactacggcggctccggggctccggggacttggcaggggagctggaggagctggaggaggagcgcgctgcctgccagggctgccgtgcccggcgcccatgggagctgttccagcatcgggccctgaggagacaggtgacaagcctcgtggttctgggcagtgccatggagctctgcgggaatgactcggtgtacgcctacgcctcctccgtgttccggaaggcaggagtgccggaagcgaagatccagtacgcgatcatcgggactgggagctgcgagctgctcacggcggttgttagttgtgtggtaatcgagagggtgggtcggcgcgtgctgctcatcggtgggtacagcctgatgacctgctgggggagcatcttcactgtggccctgtgcctgcagagctccttcccctggacactctacctggccatggcctgcatctttgccttcatcctcagctttggcattggccctgccggagtgacggggatcctggccacagagctgtttgaccagatggccaggcctgctgcctgcatggtctgcggggcgctcatgtggatcatgctcatcctggtcggcctgggatttccctttatcatggaggccttgtcccacttcctctatgtccctttccttggtgtctgtgtctgtggggccatctacactggcctgttccttcctgagaccaaaggcaagaccttccaagagatctccaaggaattacacagactcaacttccccaggcgggcccagggccccacgtggaggagcctggaggttatccagtcaacagaactctagtcccaaaggggtggccag

SEQ ID NO: 54 - protein, 496 aa, Homo sapiens
MRALRRLIQGRILLLTICAAGIGGTFQFGYNLSIINAPTLHIQEFTNETWQARTGEPLPDHLVLLMWSLIVSLYPLGGLFGALLAGPLAITLGRKKSLLVNNIFVVSAAILFGFSRKAGSFEMIMLGRLLVGVNAGVSMNIQPMYLGESAPKELRGAVAMSSAIFTALGIVMGQVVGLRELLGGPQAWPLLLASCLVPGALQLASLPLLPESPRYLLIDCGDTEACLAALRRLRGSGDLAGELEELEEERAACQGCRARRPWELFQHRALRRQVTSLVVLGSAMELCGNDSVYAYASSVFRKAGVPEAKIQYAIIGTGSCELLTAVVSCVVIERVGRRVLLIGGYSLMTCWGSIFTVALCLQSSFPWTLYLAMACIFAFILSFGIGPAGVTGILATELFDQMARPAACMVCGALMWIMLILVGLGFPFIMEALSHFLYVPFLGVCVCGAIYTGLFLPETKGKTFQEISKELHRLNFPRRAQGPTWRSLEVIQSTEL

SEQ ID NO: 55 - DNA, 2223 bp, Homo sapiens
  CDS (106)..(1959)
acactcttctttagcatgctattatggggaaagtgaccactcctgggagcgggggtggtcggggcggtttggtggcggggaagcggctgtaacttctacgtgaccatggtacctgttgaaaacaccgagggccccagtctgctgaaccagaaggggacagccgtggagacggagggcagcggcagccggcatcctccctgggcgagaggctgcggcatgtttaccttcctgtcatctgtcactgctgctgtcagtggcctcctggtgggttatgaacttgggatcatctctggggctcttcttcagatcaaaaccttattagccctgagctgccatgagcaggaaatggttgtgagctccctcgtcattggagccctccttgcctcactcaccggaggggtcctgatagacagatatggaagaaggacagcaatcatcttgtcatcctgcctgcttggactcggaagcttagtcttgatcctcagtttatcctacacggttcttatagtgggacgcattgccataggggtctccatctccctctcttccattgccacttgtgtttacatcgcagagattgctcctcaacacagaagaggccttcttgtgtcactgaatgagctgatgattgtcatcggcattctttctgcctatatttcaaattacgcatttgccaatgttttccatggctggaagtacatgtttggtcttgtgattcccttgggagttttgcaagcaattgcaatgtattttcttcctccaagccctcggtttctggtgatgaaaggacaagagggagctgctagcaaggttcttggaaggttaagagcactctcagatacaactgaggaactcactgtgatcaaatcctccctgaaagatgaatatcagtacagtttttgggatctgtttcgttcaaaagacaacatgcggacccgaataatgataggactaacactagtattttttgtacaaatcactggccaaccaaacatattgttctatgcatcaactgttttgaagtcagttggatttcaaagcaatgaggcagctagcctcgcctccactggggttggagtcgtcaaggtcattagcaccatccctgccactcttcttgtagaccatgttggcagcaaaacattcctctgcattggctcctctgtgatggcagcttcgttggtgaccatgggcatcgtaaatctcaacatccacatgaacttcacccatatctgcagaagccacaattctatcaaccagtccttggatgagtctgtgatttatggaccaggaaacctgtcaaccaacaacaatactctcagagaccacttcaaagggatttcttcccatagcagaagctcactcatgcccctgagaaatgatgtggataagagaggggagacgacctcagcatccttgctaaatgctggattaagccacactgaataccagatagtcacagaccctggggacgtcccagcttttttgaaatggctgtccttagccagcttgcttgtttatgttgctgctttttcaattggtctaggaccaatgccctggctggtgctcagcgagatctttcctggtgggatcagaggacgagccatggctttaacttctagcatgaactggggcatcaatctcctcatctcgctgacatttttgactgtaactgatcttattggcctgccatgggtgtgctttatatatacaatcatgagtctagcatccctgctttttgttgttatgtttatacctgagacaaagggatgctctttggaacaaatatcaatggagctagcaaaagtgaactatgtgaaaaacaacatttgttttatgagtcatcaccaagaagaattagtgccaaaacagcctcaaaaaagaaaaccccaggagcagctcttggagtgtaacaagctgtgtggtaggggccaatccaggcagctttctccagagacctaatggcctcaacaccttctgaacgtggatagtgccagaacacttaggagggtgtctttggaccaatgcatagttgcgactcctgtgctctcttttcagtgtcatggaactggttttgaagagacactctgaaatgataaagacagcctttaatccccctcctccccagaaggaacctcaaaaggtagatgaggtacaaggtcctaagtgatctctttttctgagcaggatatcaggcaaaaaaaaaaaaaaaaaaaaaaaaaaaaa

SEQ ID NO: 56 - protein, 617 aa, Homo sapiens
MVPVENTEGPSLLNQKGTAVETEGSGSRHPPWARGCGMFTFLSSVTAAVSGLLVGYELGIISGALLQIKTLLALSCHEQEMVVSSLVIGALLASLTGGVLIDRYGRRTAIILSSCLLGLGSLVLILSLSYTVLIVGRIAIGVSISLSSIATCVYIAEIAPQHRRGLLVSLNELMIVIGILSAYISNYAFANVFHGWKYMFGLVIPLGVLQAIAMYFLPPSPRFLVMKGQEGAASKVLGRLRALSDTTEELTVIKSSLKDEYQYSFWDLFRSKDNMRTRIMIGLTLVFFVQITGQPNILFYASTVLKSVGFQSNEAASLASTGVGVVKVISTIPATLLVDHVGSKTFLCIGSSVMAASLVTMGIVNLNIHMNFTHICRSHNSINQSLDESVIYGPGNLSTNNNTLRDHFKGISSHSRSSLMPLRNDVDKRGETTSASLLNAGLSHTEYQIVTDPGDVPAFLKWLSLASLLVYVAAFSIGLGPMPWLVLSEIFPGGIRGRAMALTSSMNWGINLLISLTFLTVTDLIGLPWVCFIYTIMSLASLLFVVMFIPETKGCSLEQISMELAKVNYVKNNICFMSHHQEELVPKQPQKRKPQEQLLECNKLCGRGQSRQLSPET

SEQ ID NO: 57 - DNA, 3261 bp, Artificial sequence
  GLUT 13
  CDS (109)..(1998)
  VARIANT 227: Xaa = Ala, Thr
  VARIANT 436: Xaa = Tyr, Phe
ctgccggcccgcggagccgcgtccccccgggcagccccgggcccctgccctatgtcccgcaaggcaagcgagaatgtggagtacacgctgcggagcctgagcagcctgatgggcgagcggcgcaggaagcagccggagccggacgcggcgagcgcggccggggagtgcagcctcctggctgccgccgaatcgagcaccagcctgcagagcgcgggcgcgggcggcggcggcgtcggggacctggagcgcgcggcgcggcggcagttccagcaggacgagacccccgccttcgtgtacgtggtggccgtcttctccgcgctgggcggcttcctgtttggctatgacaccggggtggtgtcaggggccatgctgctgctcaagcggcagctcagtctggacgcgctgtggcaggagctgctggtgtccagcacggtgggggcggctgccgtctcggcgctggccggaggcgccctcaacggcgtcttcggccgccgcgctgccatcctcctggccagtgccctcttcaccgccggctccgcggtgctggctgcggccaacaacaaggagacactgctcgccggccgcctggtcgtgggactcggcatcggcattgcttctatgacagtgccagtgtacattgcggaggtctcaccacccaatttaagaggccgattagtcaccattaataccctcttcatcacaggagggcagttctttgcaagtgttgttgatggagccttcagttatctccagaaggatggatggaggtacatgttgggacttgcarcagttccggcggttatacagttttttggctttctctttttgcctgaaagccctcgatggcttattcagaaaggacagactcagaaggcccgtagaattttatctcagatgcgtggtaaccagaccattgatgaggaatatgatagcatcaaaaacaacattgaagaggaggaaaaagaggttggctcagctggacctgtgatctgcagaatgctgagttatcccccaactcgccgagctttaattgtgggttgtggcctacaaatgttccagcagctctcaggcattaacaccatcatgtactacagtgcaaccattctgcagatgtctggtgttgaagatgatagacttgcaatatggctggcttcagttacagccttcacaaatttcattttcacacttgtgggagtctggcttgttgagaaggtgggccgcagaaagcttacctttggtagtttagcaggtaccaccgtagcactcattattcttgccttgggatttgtgctatcagcccaagtttccccacgcatcacttttaagccaatagctccgtcaggtcagaacgccacttgcacaagatacagttactgtaatgaatgtatgttggatccagactgcggtttctgctwcaagatgaacaaatcaactgtcattgactcctcctgtgttccagttaataaagcatctacaaatgaggcagcctggggcaggtgtgaaaatgaaaccaagttcaaaacagaagatatattttgggcttacaatttctgccctactccatactcctggactgcacttctgggccttattttatatcttgtcttctttgcacctggaatgggaccaatgccttggactgtgaattctgaaatatatcccctttgggcaagaagtacaggaaatgcatgttcatctggaataaactggattttcaatgtcctggtttcactaacatttttacacacagcagagtatcttacatactatggagctttcttcctctatgctggatttgctgctgtgggactccttttcatctatggctgtcttcctgagaccaaaggcaaaaaattagaggaaattgaatcactctttgacaacaggctatgtacatgtggcacttcagattctgatgaagggagatatattgaatatattcgggtaaagggaagtaactatcatctttctgacaatgatgcttctgatgtggaataattttcagctgctcatatatttagttatttaaacaaactggggggagaagaacagcaattggtgacttcactgccctgcttctaatctggttctttccacagcctagttttgattgacttcatattctagaatacttgattaggaggaagatacaaccatgatgactttttttttccacaaggaacaatattttaaaaaatatttacagagattttaatctaataattcttaagcaaatgtgtgtaatgccttcctgaaatagtctaaaatgaatattgtacccagtgacttcagtggtatccttttttcctaagaccatttataattattagtggcaacagagtcagtgctaatctagccaaattacatatgtataatatatttataaaggattctgggagatggtccaagggtgttctgtgtcaaaagatggcctattggccctcagttttcctacagagtagtggcttatctctgatcagctgttacaaactaaattccatgtaagctttcatcaacaaattccaaagtgcctcctacaagggcacagctgtccgtatctcctttggattccatatttttgtttctctccaattcagatattgggagttcttcagatactgactctgcacactatcttttgaacattatgaaaatataatctgtcgggttgttttcatacttcctttttttgcatcagtgataaggcatacatttgcgcagatgagctaggaagttttgagaagtaccttttatgttcagaaagtaacagattaaaggtttttatgggttccaaattggttagtcacttaggtatgcaattataaatgcaaaatagtgccagatctttaactgtatatcagggtcagtttttgttcatgcccaaatcactacaccttcctcattggccttggagtcctactattttgtgccttcatttgtgttacatacatagctgcaagcaaaccatttttcccctttcttttattcaaacaataatttttgaaacaaaaaagaggaaggaaatcagtggcagaaataatcctgctgttattggtgtttgtttaataaaaataatgggacttttttcttaactttttattagctcttcctaagggaaatgtcacatattattatttaattgtacttgtctttttttactttaagagcataaactcgtttttattttgcacacttttctcattttcctgagaatttaccagaaaaaaaaagatacatagatttgtctctgtgtttttctta

SEQ ID NO: 58 - protein, 629 aa, Artificial sequence
  misc_feature (227)..(227): The ′Xaa′ at location 227 stands for Ala, or Thr.
  misc_feature (436)..(436): The ′Xaa′ at location 436 stands for Tyr, or Phe.
  Synthetic Construct
MGERRRKQPEPDAASAAGECSLLAAAESSTSLQSAGAGGGGVGDLERAARRQFQQDETPAFVYVVAVFSALGGFLFGYDTGVVSGAMLLLKRQLSLDALWQELLVSSTVGAAAVSALAGGALNGVFGRRAAILLASALFTAGSAVLAAANNKETLLAGRLVVGLGIGIASMTVPVYIAEVSPPNLRGRLVTINTLFITGGQFFASVVDGAFSYLQKDGWRYMLGLAXVPAVIQFFGFLFLPESPRWLIQKGQTQKARRILSQMRGNQTIDEEYDSIKNNIEEEEKEVGSAGPVICRMLSYPPTRRALIVGCGLQMFQQLSGINTIMYYSATILQMSGVEDDRLAIWLASVTAFTNFIFTLVGVWLVEKVGRRKLTFGSLAGTTVALIILALGFVLSAQVSPRITFKPIAPSGQNATCTRYSYCNECMLDPDCGFCXKMNKSTVIDSSCVPVNKASTNEAAWGRCENETKFKTEDIFWAYNFCPTPYSWTALLGLILYLVFFAPGMGPMPWTVNSEIYPLWARSTGNACSSGINWIFNVLVSLTFLHTAEYLTYYGAFFLYAGFAAVGLLFIYGCLPETKGKKLEEIESLFDNRLCTCGTSDSDEGRYIEYIRVKGSNYHLSDNDASDVE

SEQ ID NO: 59 - DNA, 2118 bp, Homo sapiens
  CDS (110)..(1603)
agcggggctgagcgaagccgcggggcccaaccgcagtcgcggggtctgggaggaagcagtaccttgaagagaaattggagagggagtcaattcctaggatagcagagagatggacaacagacagaatgtcaccccagctctgatctttgccatcacagttgctacaatcggctctttccagtttggctacaacactggggtcatcaatgctcctgagacgatcataaaggaatttatcaataaaactttgacggacaaggcaaatgcccctccctctgaggtgctgctcacgaatctctggtccttgtctgtggccatattttccgtcgggggtatgatcggctccttttccgtcggactctttgttaaccgctttggcaggcgcaattcaatgctgattgtcaacctgttggctgccactggtggctgccttatgggactgtgtaaaatagctgagtcagttgaaatgctgatcctgggccgcttggttattggcctcttctgcggactctgcacaggttttgtgcccatgtacattggagagatctcgcctactgccctgaggggtgcctttggcactctcaaccagctgggcatagttattggaattctggtggcccagatctttggtctggaactcatccttgggtctgaagagctatggccggtgctattaggctttaccatccttccagctatcctgcaaagtgcagcccttccatgttgccctgaaagtcccagatttttgctcattaacagaaaaaaagaggagaatgctacgcggatcctccagcggttgtggggcacccaggatgtatcccaagacatccaggagatgaaagatgagagtgcaaggatgtcacaagaaaagcaagtcaccgtgctggagctctttagagtgtccagctaccgacagcccatcatcatttccattgtgctccagctctctcagcagctctctgggatcaatgctgtgttctattactcaacaggaatcttcaaggatgcaggtgttcaacagcccatctatgccaccatcagcgcgggtgtggttaatactatcttcactttactttctctatttctggtggaaagggcaggaagaaggactctgcatatgataggccttggagggatggctttttgttccacgctcatgactgtttctttgttattaaagaatcactataatgggatgagctttgtctgtattggggctatcttggtctttgtggcctgttttgaaattggaccaggccccattccctggtttattgtggccgaactcttcagccagggcccccgcccagctgcgatggcagtggccggctgctccaactggacctccaacttcctagtcggattgctcttcccctctgctgcttactatttaggagcctacgtttttattatcttcaccggcttcctcattaccttcttggcctttaccttcttcaaagtccctgagacccgtggcaggacttttgaggatatcacacgggcctttgaagggcaggcacacggtgcagatagatctgggaaggacggcgtcatggggatgaacagcatcgagcctgctaaggagaccaccaccaatgtctaagtcatgcctccttccacctccctcccggcatgggaaagccacctctccctcaacaagggagagactttatcaggatgaacccaggactgcttctgaatgctgctacttgatttctttctcatcccacgcactccatgagcaccccaaggctgcagtttgttggatcttcaatggctttttaaattttatttcctggacatcctcttctgcttaggagagaccgagtgaacctaccttcatttcaggagggattggccgcttggcacatgacaactttgccagcttttcctcccttgggttctgatattgccgcactagaggatataggagaggaaaagtaaggtgcagttgccccaacctcagacttaccaggaagcagatacatatgagtgtggaagccggagggtgtttatgtaagagcaccttcctcacttccatacagctctacgcggcaaattaacttgagttttatttatcttatcctctggtttaattacataaatatttatttttta

SEQ ID NO: 60 - protein, 497 aa, Homo sapiens
MDNRQNVTPALIFAITVATIGSFQFGYNTGVINAPETIIKEFINKTLTDKANAPPSEVLLTNLWSLSVAIFSVGGMIGSFSVGLFVNRFGRRNSMLIVNLLAATGGCLMGLCKIAESVEMLILGRLVIGLFCGLCTGFVPMYIGEISPTALRGAFGTLNQLGIVIGILVAQIFGLELILGSEELWPVLLGFTILPAILQSAALPCCPESPRFLLINRKKEENATRILQRLWGTQDVSQDIQEMKDESARMSQEKQVTVLELFRVSSYRQPIIISIVLQLSQQLSGINAVFYYSTGIFKDAGVQQPIYATISAGVVNTIFTLLSLFLVERAGRRTLHMIGLGGMAFCSTLMTVSLLLKNHYNGMSFVCIGAILVFVACFEIGPGPIPWFIVAELFSQGPRPAAMAVAGCSNWTSNFLVGLLFPSAAYYLGAYVFIIFTGFLITFLAFTFFKVPETRGRTFEDITRAFEGQAHGADRSGKDGVMGMNSIEPAKETTTNV

SEQ ID NO: 61 - DNA, 6126 bp, Homo sapiens
  CDS (133)..(4572)
aattggaagcaaatgacatcacagcaggtcagagaaaaagggttgagcggcaggcacccagagtagtaggtctttggcattaggagcttgagcccagacggccctagcagggaccccagcgcccgagagaccatgcagaggtcgcctctggaaaaggccagcgttgtctccaaactttttttcagctggaccagaccaattttgaggaaaggatacagacagcgcctggaattgtcagacatataccaaatcccttctgttgattctgctgacaatctatctgaaaaattggaaagagaatgggatagagagctggcttcaaagaaaaatcctaaactcattaatgcccttcggcgatgttttttctggagatttatgttctatggaatctttttatatttaggggaagtcaccaaagcagtacagcctctcttactgggaagaatcatagcttcctatgacccggataacaaggaggaacgctctatcgcgatttatctaggcataggcttatgccttctctttattgtgaggacactgctcctacacccagccatttttggccttcatcacattggaatgcagatgagaatagctatgtttagtttgatttataagaagactttaaagctgtcaagccgtgttctagataaaataagtattggacaacttgttagtctcctttccaacaacctgaacaaatttgatgaaggacttgcattggcacatttcgtgtggatcgctcctttgcaagtggcactcctcatggggctaatctgggagttgttacaggcgtctgccttctgtggacttggtttcctgatagtccttgccctttttcaggctgggctagggagaatgatgatgaagtacagagatcagagagctgggaagatcagtgaaagacttgtgattacctcagaaatgattgaaaatatccaatctgttaaggcatactgctgggaagaagcaatggaaaaaatgattgaaaacttaagacaaacagaactgaaactgactcggaaggcagcctatgtgagatacttcaatagctcagccttcttcttctcagggttctttgtggtgtttttatctgtgcttccctatgcactaatcaaaggaatcatcctccggaaaatattcaccaccatctcattctgcattgttctgcgcatggcggtcactcggcaatttccctgggctgtacaaacatggtatgactctcttggagcaataaacaaaatacaggatttcttacaaaagcaagaatataagacattggaatataacttaacgactacagaagtagtgatggagaatgtaacagccttctgggaggagggatttggggaattatttgagaaagcaaaacaaaacaataacaatagaaaaacttctaatggtgatgacagcctcttcttcagtaatttctcacttcttggtactcctgtcctgaaagatattaatttcaagatagaaagaggacagttgttggcggttgctggatccactggagcaggcaagacttcacttctaatgatgattatgggagaactggagccttcagagggtaaaattaagcacagtggaagaatttcattctgttctcagttttcctggattatgcctggcaccattaaagaaaatatcatcggtgtttcctatgatgaatatagatacagaagcgtcatcaaagcatgccaactagaagaggacatctccaagtttgcagagaaagacaatatagttcttggagaaggtggaatcacactgagtggaggtcaacgagcaagaatttctttagcaagagcagtatacaaagatgctgatttgtatttattagactctccttttggatacctagatgttttaacagaaaaagaaatatttgaaagctgtgtctgtaaactgatggctaacaaaactaggattttggtcacttctaaaatggaacatttaaagaaagctgacaaaatattaattttgaatgaaggtagcagctatttttatgggacattttcagaactccaaaatctacagccagactttagctcaaaactcatgggatgtgattctttcgaccaatttagtgcagaaagaagaaattcaatcctaactgagaccttacaccgtttctcattagaaggagatgctcctgtctcctggacagaaacaaaaaaacaatcttttaaacagactggagagtttggggaaaaaaggaagaattctattctcaatccaatcaactctatacgaaaattttccattgtgcaaaagactcccttacaaatgaatggcatcgaagaggattctgatgagcctttagagagaaggctgtccttagtaccagattctgagcagggagaggcgatactgcctcgcatcagcgtgatcagcactggccccacgcttcaggcacgaaggaggcagtctgtcctgaacctgatgacacactcagttaaccaaggtcagaacattcaccgaaagacaacagcatccacacgaaaagtgtcactggcccctcaggcaaacttgactgaactggatatatattcaagaaggttatctcaagaaactggcttggaaataagtgaagaaattaacgaagaagacttaaaggagtgcctttttgatgatatggagagcataccagcagtgactacatggaacacataccttcgatatattactgtccacaagagcttaatttttgtgctaatttggtgcttagtaatttttctggcagaggtggctgcttctttggttgtgctgtggctccttggaaacactcctcttcaagacaaagggaatagtactcatagtagaaataacagctatgcagtgattatcaccagcaccagttcgtattatgtgttttacatttacgtgggagtagccgacactttgcttgctatgggattcttcagaggtctaccactggtgcatactctaatcacagtgtcgaaaattttacaccacaaaatgttacattctgttcttcaagcacctatgtcaaccctcaacacgttgaaagcaggtgggattcttaatagattctccaaagatatagcaattttggatgaccttctgcctcttaccatatttgacttcatccagttgttattaattgtgattggagctatagcagttgtcgcagttttacaaccctacatctttgttgcaacagtgccagtgatagtggcttttattatgttgagagcatatttcctccaaacctcacagcaactcaaacaactggaatctgaaggcaggagtccaattttcactcatcttgttacaagcttaaaaggactatggacacttcgtgccttcggacggcagccttactttgaaactctgttccacaaagctctgaatttacatactgccaactggttcttgtacctgtcaacactgcgctggttccaaatgagaatagaaatgatttttgtcatcttcttcattgctgttaccttcatttccattttaacaacaggagaaggagaaggaagagttggtattatcctgactttagccatgaatatcatgagtacattgcagtgggctgtaaactccagcatagatgtggatagcttgatgcgatctgtgagccgagtctttaagttcattgacatgccaacagaaggtaaacctaccaagtcaaccaaaccatacaagaatggccaactctcgaaagttatgattattgagaattcacacgtgaagaaagatgacatctggccctcagggggccaaatgactgtcaaagatctcacagcaaaatacacagaaggtggaaatgccatattagagaacatttccttctcaataagtcctggccagagggtgggcctcttgggaagaactggatcagggaagagtactttgttatcagcttttttgagactactgaacactgaaggagaaatccagatcgatggtgtgtcttgggattcaataactttgcaacagtggaggaaagcctttggagtgataccacagaaagtatttattttttctggaacatttagaaaaaacttggatccctatgaacagtggagtgatcaagaaatatggaaagttgcagatgaggttgggctcagatctgtgatagaacagtttcctgggaagcttgactttgtccttgtggatgggggctgtgtcctaagccatggccacaagcagttgatgtgcttggctagatctgttctcagtaaggcgaagatcttgctgcttgatgaacccagtgctcatttggatccagtaacataccaaataattagaagaactctaaaacaagcatttgctgattgcacagtaattctctgtgaacacaggatagaagcaatgctggaatgccaacaatttttggtcatagaagagaacaaagtgcggcagtacgattccatccagaaactgctgaacgagaggagcctcttccggcaagccatcagcccctccgacagggtgaagctctttccccaccggaactcaagcaagtgcaagtctaagccccagattgctgctctgaaagaggagacagaagaagaggtgcaagatacaaggctttagagagcagcataaatgttgacatgggacatttgctcatggaattggagctcgtgggacagtcacctcatggaattggagctcgtggaacagttacctctgcctcagaaaacaaggatgaattaagtttttttttaaaaaagaaacatttggtaaggggaattgaggacactgatatgggtcttgataaatggcttcctggcaatagtcaaattgtgtgaaaggtacttcaaatccttgaagatttaccacttgtgttttgcaagccagattttcctgaaaacccttgccatgtgctagtaattggaaaggcagctctaaatgtcaatcagcctagttgatcagcttattgtctagtgaaactcgttaatttgtagtgttggagaagaactgaaatcatacttcttagggttatgattaagtaatgataactggaaacttcagcggtttatataagcttgtattcctttttctctcctctccccatgatgtttagaaacacaactatattgtttgctaagcattccaactatctcatttccaagcaagtattagaataccacaggaaccacaagactgcacatcaaaatatgccccattcaacatctagtgagcagtcaggaaagagaacttccagatcctggaaatcagggttagtattgtccaggtctaccaaaaatctcaatatttcagataatcacaatacatcccttacctgggaaagggctgttataatctttcacaggggacaggatggttcccttgatgaagaagttgatatgccttttcccaactccagaaagtgacaagctcacagacctttgaactagagtttagctggaaaagtatgttagtgcaaattgtcacaggacagcccttctttccacagaagctccaggtagagggtgtgtaagtagataggccatgggcactgtgggtagacacacatgaagtccaagcatttagatgtataggttgatggtggtatgttttcaggctagatgtatgtacttcatgctgtctacactaagagagaatgagagacacactgaagaagcaccaatcatgaattagttttatatgcttctgttttataattttgtgaagcaaaattttttctctaggaaatatttattttaataatgtttcaaacatatattacaatgctgtattttaaaagaatgattatgaattacatttgtataaaataatttttatatttgaaatattgactttttatggcactagtatttttatgaaatattatgttaaaactgggacaggggagaacctagggtgatattaaccaggggccatgaatcaccttttggtctggagggaagccttggggctgatcgagttgttgcccacagctgtatgattcccagccagacacagcctcttagatgcagttctgaagaagatggtaccaccagtctgactgtttccatcaagggtacactgccttctcaactccaaactgactcttaagaagactgcattatatttattactgtaagaaaatatcacttgtcaataaaatccatacatttgtgt

SEQ ID NO: 62 - protein, 1479 aa, Homo sapiens
MQRSPLEKASVVSKLFFSWTRPILRKGYRQRLELSDIYQIPSVDSADNLSEKLEREWDRELASKKNPKLINALRRCFFWRFMFYGIFLYLGEVTKAVQPLLLGRIIASYDPDNKEERSIAIYLGIGLCLLFIVRTLLLHPAIFGLHHIGMQMRIAMFSLIYKKTLKLSSRVLDKISIGQLVSLLSNNLNKFDEGLALAHFVWIAPLQVALLMGLIWELLQASAFCGLGFLIVLALFQAGLGRMMMKYRDQRAGKISERLVITSEMIENIQSVKAYCWEEAMEKMIENLRQTELKLTRKAAYVRYFNSSAFFFSGFFVVFLSVLPYALIKGIILRKIFTTISFCIVLRMAVTRQFPWAVQTWYDSLGAINKIQDFLQKQEYKTLEYNLTTTEVVMENVTAFWEEGFGELFEKAKQNNNNRKTSNGDDSLFFSNFSLLGTPVLKDINFKIERGQLLAVAGSTGAGKTSLLMMIMGELEPSEGKIKHSGRISFCSQFSWIMPGTIKENIIGVSYDEYRYRSVIKACQLEEDISKFAEKDNIVLGEGGITLSGGQRARISLARAVYKDADLYLLDSPFGYLDVLTEKEIFESCVCKLMANKTRILVTSKMEHLKKADKILILNEGSSYFYGTFSELQNLQPDFSSKLMGCDSFDQFSAERRNSILTETLHRFSLEGDAPVSWTETKKQSFKQTGEFGEKRKNSILNPINSIRKFSIVQKTPLQMNGIEEDSDEPLERRLSLVPDSEQGEAILPRISVISTGPTLQARRRQSVLNLMTHSVNQGQNIHRKTTASTRKVSLAPQANLTELDIYSRRLSQETGLEISEEINEEDLKECLFDDMESIPAVTTWNTYLRYITVHKSLIFVLIWCLVIFLAEVAASLVVLWLLGNTPLQDKGNSTHSRNNSYAVIITSTSSYYVFYIYVGVADTLLAMGFFRGLPLVHTLITVSKILHHKMLHSVLQAPMSTLNTLKAGGILNRFSKDIAILDDLLPLTIFDFIQLLLIVIGAIAVVAVLQPYIFVATVPVIVAFIMLRAYFLQTSQQLKQLESEGRSPIFTHLVTSLKGLWTLRAFGRQPYFETLFHKALNLHTANWFLYLSTLRWFQMRIEMIFVIFFIAVTFISILTTGEGEGRVGIILTLAMNIMSTLQWAVNSSIDVDSLMRSVSRVFKFIDMPTEGKPTKSTKPYKNGQLSKVMIIENSHVKKDDIWPSGGQMTVKDLTAKYTEGGNAILENISFSISPGQRVGLLGRTGSGKSTLLSAFLRLLNTEGEIQIDGVSWDSITLQQWRKAFGVIPQKVFIFSGTFRKNLDPYEQWSDQEIWKVADEVGLRSVIEQFPGKLDFVLVDGGCVLSHGHKQLMCLARSVLSKAKILLLDEPSAHLDPVTYQIIRRTLKQAFADCTVILCEHRIEAMLECQQFLVIEENKVRQYDSIQKLLNERSLFRQAISPSDRVKLFPHRNSSKCKSKPQIAALKEETEEEVQDTRL

SEQ ID NO: 63 - DNA, 36 bp, Artificial sequence
  Oligonucleotide encoding HA tag
tgagatcgattatccttatgatgttcctgattatgg

SEQ ID NO: 64 - DNA, 36 bp, Artificial sequence
  oligonucleotide encoding HA tag
tcagcataatcaggaacatcataaggataatcgatc

(19) United States
(12) Patent Application Publication
(10) Pub. No.: US 2007/0141635 A1
(43) Pub. Date: Jun. 21, 2007

(54) Novel translocation assay

(76) Inventor: David James, Clontarf (AU)

Correspondence Address: KNOBBE MARTENS OLSON & BEAR LLP, 2040 MAIN STREET, FOURTEENTH FLOOR, IRVINE, CA, 92614, US

(21) Appl. No.: 10/567,894

(22) PCT Filed: Aug. 9, 2004
(86) PCT No.: PCT/AU04/01057

§ 371 Date: Feb. 5, 2007

(30) Foreign Application Priority Data
Aug. 8, 2003 (AU) 2003904237

(51) Int. Cl. G01N 33/567

(52) U.S. Cl. 435/7.2

(57) ABSTRACT

The present invention relates to a novel in vitro assay for determining the level of a protein, in particular, a membrane transport protein that is located at the plasma membrane of a cell compared to the level of the protein in the cell. The process of the invention is also useful for determining the level of recycling of a membrane transport protein. The present invention additionally provides a process for identifying an agent that modulates the translocation of a protein, in particular, a membrane transport protein, to the plasma membrane and, as a consequence, the activity of that protein.

### FIELD OF THE INVENTION

The present invention relates to a novel in vitro assay for determining the level of a protein, in particular, a membrane transport protein that is located at the plasma membrane of a cell compared to the level of the protein in the cell. In one embodiment, the present invention provides a method for identifying an agent that modulates the translocation of a protein, in particular, a membrane transport protein, to the plasma membrane and, as a consequence, the activity of that protein.

### BACKGROUND OF THE INVENTION

General

This specification contains nucleotide and amino acid sequence information prepared using Patent in Version 3.1, presented herein after the claims. Each nucleotide sequence is identified in the sequence listing by the numeric indicator <210> followed by the sequence identifier (e.g. <210>1, <210>2, <210>3, etc). The length and type of sequence (DNA, protein (PRT), etc), and source organism for each nucleotide sequence, are indicated by information provided in the numeric indicator fields <211>, <212> and <213>, respectively. Nucleotide sequences referred to in the specification are defined by the term “SEQ ID NO:”, followed by the sequence identifier (eg. SEQ ID NO: 1 refers to the sequence in the sequence listing designated as <400>1).

The designation of nucleotide residues referred to herein are those recommended by the IUPAC-IUB Biochemical Nomenclature Commission-, wherein A represents Adenine, C represents Cytosine, G represents Guanine, T represents thymine, Y represents a pyrimidine residue, R represents a purine residue, M represents Adenine or Cytosine, K represents Guanine or Thymine, S represents Guanine or Cytosine, W represents Adenine or Thymine, H represents a nucleotide other than Guanine, B represents a nucleotide other than Adenine, V represents a nucleotide other than Thymine, D represents a nucleotide other than Cytosine and N represents any nucleotide residue.

As used herein the term “derived from” shall be taken to indicate that a specified integer may be obtained from a particular source albeit not necessarily directly from that source.

Throughout this specification, unless the context requires otherwise, the word “comprise”, or variations such as “comprises” or “comprising”, will be understood to imply the inclusion of a stated step or element or integer or group of steps or elements or integers but not the exclusion of any other step or element or integer or group of elements or integers.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Each embodiment described herein is to be applied mutatis mutandis to each and every other embodiment unless specifically stated otherwise.

Those skilled in the art will appreciate that the invention described herein is susceptible to variations and modifications other than those specifically described. It is to be understood that the invention includes all such variations, and modifications. The invention also includes all of the steps, features, compositions and compounds referred to or indicated in this specification, individually or collectively, and any and all combinations or any two or more of said steps or features.

The present invention is not to be limited in scope by the specific embodiments described herein, which are intended for the purpose of exemplification only. Functionally-equivalent products, compositions and methods are clearly within the scope of the invention, as described herein.

The present invention is performed without undue experimentation using, unless otherwise indicated, conventional techniques of molecular biology, microbiology, virology, recombinant DNA technology, peptide synthesis in solution, solid phase peptide synthesis, and immunology. Such procedures are described, for example, in the following texts that are incorporated by reference:

Sambrook, Fritsch & Maniatis, Molecular Cloning: A Laboratory Manual, Cold Spring Harbor Laboratories, New York, Second Edition (1989), whole of Vols I, II, and III;

DNA Cloning: A Practical Approach, Vols. I and II (D. N. Glover, ed., 1985), IRL Press, Oxford, whole of text;

Oligonucleotide Synthesis: A Practical Approach (M. J. Gait, ed., 1984) IRL Press, Oxford, whole of text, and particularly the papers therein by Gait, pp1-22; Atkinson et al., pp35-81; Sproat et al., pp 83-115; and Wu et al., pp 135-151;

Nucleic Acid Hybridization: A Practical Approach (B. D. Hames & S. J. Higgins, eds., 1985) IRL Press, Oxford, whole of text;

Animal Cell Culture: Practical Approach, Third Edition (John R. W. Masters, ed., 2000), ISBN 0199637970, whole of text;

Imnobilized Cells and Enzymes: A Practical Approach (1986) IRL Press, Oxford, whole of text;

Perbal, B., A Practical Guide to Molecular Cloning (1984);

Methods In Enzymology (S. Colowick and N. Kaplan, eds., Academic Press, Inc.), whole of series;

J. F. Ramalho Ortigão, “The Chemistry of Peptide Synthesis” In: Knowledge database of Access to Virtual Laboratory website (Interactiva, Germany);

Sakakibara, D., Teichman, J., Lien, E. Land Fenichel, R. L. (1976). Biochem. Biophys. Res. Commun. 73 336-342

Merrifield, R. B. (1963). J. Am. Chem. Soc. 85, 2149-2154.

Barany, G. and Merrifield, R. B. (1979) in The Peptides (Gross, E. and Meienhofer, J. eds.), vol. 2, pp. 1-284, Academic Press, New York.

Wünsch, E., ed. (1974) Synthese von Peptiden in Houben-Weyls Metoden der Organischen Chemie (Müler, E., ed.), vol. 15, 4th edn., Parts 1 and 2, Thieme, Stuttgart.

Bodanszky, M. (1984) Principles of Peptide Synthesis, Springer-Verlag, Heidelberg. Bodanszky, M. & Bodanszky, A. (1984) The Practice of Peptide Synthesis, Springer-Verlag, Heidelberg.

Bodanszky, M. (1985) Int. J. Peptide Protein Res. 25, 449474.

Handbook of Experimental Immunology, Vols. I-IV (D. M. Weir and C. C. Blackwell, eds., 1986, Blackwell Scientific Publications).

Description of the Related Art

An important activity performed by any cell is the transport of materials across the plasma membrane. This activity is essential for the survival of all organisms, from simple unicellular organisms, e.g. bacteria, to complex multicellular organisms, e.g. humans. Not only does membrane transport facilitate the uptake of, for example, nutrients and ions, but also the excretion of waste products, and the secretion of signaling molecules.

The process of membrane transport itself is performed by a large class of proteins known as transporters” “membrane transporters” “membrane transport proteins”. A number of these proteins function by forming protein channels in the plasma membrane of a cell. This class of proteins includes a vast number of proteins that are related by their ability to transport other molecules across a cell membrane. It is hypothesized that the number of proteins involved in membrane transport constitute approximately 5% to 10% of known open reading frames in most sequenced genomes.

Membrane transport proteins are generally localized both intracellularly and within the plasma membrane. However, as the membrane-localized form is capable of transport activity, the amount of any membrane transport protein present in the plasma membrane limits the transport of substrates (both naturally-occurring substrates and small molecules) into and/or outside of the cell. Exemplary membrane transport proteins include the glucose-transporters (e.g. GLUT1, GLUT4), water transporters (e.g., aquaporins) and ion transporters that transport Cl⁻, K⁺, Na⁺, Cu²⁺ or S0₄²⁻ ions, amongst others (e.g. cystic fibrosis transmembrane regulator (CFTR), pendrin, human ether-a-go-go (HERG)). As will be known to those skilled in the art, membrane transport proteins may function in the transport of multiple substrates for example, in the same direction (e.g., symport) across the plasma membrane or in the opposite direction (eg., antiport) across the plasma membrane.

Cells utilize a number of transport mechanisms, all of which are controlled by transport proteins.

Facilitated diffusion utilizes membrane protein channels to allow charged molecules (which otherwise could not diffuse across a plasma membrane) to freely move across a plasma membrane. For example, K+, Na+, and Cl− are transported across a plasma membrane by such membrane protein channels.

Facilitative transport molecules convey molecules, such as, for example, sugars down a concentration gradient, i.e. from a region of high concentration of that molecule to a region of low concentration, in a process that does not require energy.

In contrast, active transport requires the expenditure of energy to transport the molecule across the membrane. Similar to facilitated transport, active transport is limited by the number of membrane transport proteins present at the membrane.

Active, or coupled, membrane transporters transport substrates against a concentration gradient in a process that either requires energy expenditure or the use of another concentration gradient. For example, sodium dependent glucose transporters couple the transport of one molecule of glucose to two molecules of sodium. Sodium ions are transported down their concentration in a process that generates sufficient free energy to transport glucose against its concentration gradient allowing for a significant increase in the concentration of glucose in a cell.

As membrane transport proteins are involved in such a variety of functions that are essential to the survival of an organism, it is not surprising that several of these proteins have been found to be associated with disease in humans. For example, several forms of hearing loss in humans are associated with mutations in genes encoding transport proteins such as, for example, connexin 26, and pendrin, a proposed sulfate transporter. Defects in ion transporters are associated with a predisposition to cardiac arrhythmia, Menke's disease, Wilson's disease, familial generalized epilepsy, benign infantile epilepsy, spinocerebellar ataxia and familial hemiplegic migraine amongst many others.

Additionally, deficiency of the water channel protein aquaporin 2 hinders its translocation to the apical surface of the cell abolishing reabsorption of water from the collecting duct and resulting in nephrogenic diabetes insipidus.

Diabetes is associated with a dysfunctional glucose uptake into muscle and fat cells due to the impaired ability of insulin to stimulate glucose transporters.

In addition to mutations that directly affect the activity of a protein, any defect that 30 inhibits the trafficking of the relevant membrane transport protein to the correct subcellular location has also been shown to be linked with human disease. For example, it has been suggested that the membrane transport protein GLUT4 is abnormally localized in type II diabetes (Bryant et al, Nature Reviews Molecular Cell Biology, 3, 267-277, 2002). In a normal cell GLUT4, which transports glucose across the plasma membrane, is thought to be almost entirely intracellular in the absence of insulin Upon the addition of insulin, GLUT4 translocates to the plasma membrane.

However, in skeletal muscle cells from some type II diabetes mellitus subjects (Kelley et al, J. Clin. Invest. 97, 2705-2713, 1996) GLUT4 translocation has been shown to be drastically reduced. These results suggest impaired glucose transport as a consequence of impaired GLUT4 translocation may play a role in insulin resistance in type II diabetes.

The most common mutations in the cystic fibrosis transmembrane regulator (CFTR) gene (the ΔF508 mutation, A1507 mutation, K464M mutation, F508R mutation, and S5491 mutation, which account for approximately 70% of CF patients) have been suggested to cause abnormal localization of the CFTR protein to the endoplasmic reticulum, where it is subsequently degraded (Cheng et al, Cell, 63(4), 827-834, 1990). Such mutant forms of the CFTR protein have been observed to be localized at the apical region of the cytosol of cells, rather than within the plasma membrane. As the CFTR protein is a chloride channel, the reduction in the amount of this channel in the membrane is associated with reduced movement of both sodium and water into the cell. The mislocalization of the CFTR protein has also been suggested as a possible causative factor in the reduced movement of sodium and water observed in the lungs and intestines of subjects suffering from cystic fibrosis.

In the case of cardiac arrhythinia, mutations have been found in the genes encoding the potassium channels, human ether-a-go-go-related gene (HERG), and KVLQT1. The HERG protein is the pore-forming subunit of the cardiac rapidly activating delayed rectifier potassium channel. In both cases, mutations in the gene encoding each protein are associated with a reduction with trafficking of the protein and, as a consequence, a reduction in the amount of the protein being integrated into the plasma membrane. As a result, cardiac cells expressing the mutant protein show reduced amplitude and altered voltage dependence of activation (Zhou et al, J. Biol. Chem., 274(44), 31123-31126, 1999).

Mutations in various other membrane transport proteins have also been suggested to cause a number of disorders due to altered or incorrect trafficldng/translocation of the mutant protein, for example, glucose-galactose malabsorption, changes in cholesterol homeostasis, and defects in the multi-drug transporter β-glycoprotein.

As membrane transport proteins are involved in several essential cellular processes, and mutations affecting the function and/or localization of these proteins are involved in the etiology of certain human diseases, there is a clear need in the art for methods of detecting mutations in these proteins and/or modulatory agents that affect their subcellular localization and/or turnover/recycling.

Known methods of determining the activity of a membrane transport protein generally involve the mere measurement of the movement of a specific substrate across a lipid bilayer, such as that found at the membrane of a cell. These methods are imprecise, as any redundancy in the transport process of interest, e.g. if a cell expresses multiple proteins that transport the same molecule, may mask or reduce the effect of a mutation of one of the constituents (i.e. transport proteins) of the process. For example, there are at least 12 hexose transporters encoded by the genes in the human genome and most mammalian cell types express more than one member of this family.

Alternatively, plasma membranes are isolated and low density microsomal fractions prepared. The membrane transport proteins are then photolabeled (e.g. bis-mannose photolabeling of GLUT4 located on the cell surface), and subsequently immunoprecipitated e.g. as described in Homan et al., J. Biol. Chem. 26:5 18172-18179 (1990).

Alternatively, plasma membrane sheets are prepared for use in microscopic analysis essentially as described in Cushman and Wardzala., J. Biol Chem. 255:47584762 (1980), or by isolation of plasma membrane sheets or lawns for use in microscopic analysis as described in Robinson, et al., J Cell Biol. 117:1181-1196 (1992).

These assays are both laborious and subject to inter-assay variability, and furthermore, are only semi-quantitative. Accordingly, the quantitative nature of these assays is limited. Furthermore, these assays are not readily adapted to high-throughput analysis, for example, for screening compounds that modulate translocation of a membrane transport protein.

Accordingly, there is a clear need in the art for a straightforward, reproducible method for the detection and estimation of the level of a membrane transport protein translocated to the plasma membrane. Preferred assays will not require sub-cellular fractionation or multiple labeling. Preferred assays will also be useful for determining mutations and/or agents that affect translocation of the membrane transport protein, for example, in a high-throughput assay.

### SUMMARY OF THE INVENTION

In work leading up to the present invention, the inventors sought to develop an assay that detects the level of a membrane transport protein incorporated into the plasma membrane of a cell compared to the total level of said membrane transport protein within the cell. Furthermore, the inventors sought to use this assay to determine the level of trafficking and/or turnover of the membrane transport protein at the plasma membrane.

For example, the present inventors have developed an assay useful for determining the level of GLUT4 translocation in a cell. The assay uses a GLUT4 protein that is labeled with a tag or marker that facilitates detection of the GLUT4. Preferably, the tag or marker is located within an extracellular domain of the GLUT4 protein. The location of the tag or marker facilitates detection of the GLUT4 protein at the plasma membrane of an intact cell. By determining the level of tagged/marked GLUT4 protein at the plasma membrane of a cell relative to the level of tagged/marked GLUT4 in the cell, the level of GLUT4 translocation is determined.

The present inventors have additionally shown that the process of the present invention is amenable to performance in 96-well and 384-well formats. Accordingly, this assay provides a high throughput screen to determine a modulator of translocation of a membrane transport protein. Such a modulator represents a candidate therapeutic for the treatment of a disease associated with translocation (e.g. aberrant translocation) of a membrane transport protein.

Furtherrnore, the present inventors have developed a model of insulin resistance observed in subjects suffering from type-II diabetes. This assay provides the basis for a screen to determine a candidate compound for the treatment of insulin resistance e.g. that associated with type-II diabetes.

The present invention provides a process for determining the level of a membrane transport protein translocated to the plasma membrane of a cell, said method comprising:
(a) determining the level of a membrane transport protein at the plasma membrane of the cell using a method comprising:
   (i) contacting the cell with a ligand that binds to an extracellular domain of the membrane transport protein for a time and under conditions sufficient for the ligand to bind to the membrane transport protein at the plasma membrane of the cell; and
   (ii) determining the level of ligand bound to the membrane transport protein;
b) (i) permeabilizing or disrupting the plasma membrane of a cell and contacting the membrane transport protein within the cell with the ligand for a time and under conditions sufficient for the ligand to bind to the membrane transport protein; and
   (ii) determining the level of ligand bound to the membrane transport protein; and
(c) comparing the level of ligand determined at (a) (ii) and (b) (ii) to determine the level of the membrane transport protein at the plasma membrane relative to the level of the membrane transport protein inside the cell.

For example, the membrane transport protein is a glucose transport (GLUT) protein.

In an example, the membrane transport protein is GLUT4, e.g., the GLUT4 comprises an amino acid sequence at least 80% identical to the amino acid sequence set forth in SEQ ID NO: 2.

In another example, the membrane transport protein is GLUT1 e.g., the GLUT1 comprises an amino acid sequence at least 80% identical to the amino acid sequence set forth in SEQ ID NO: 12.

In yet another example, the membrane transport protein is a mutant membrane transport protein having a reduced rate of recycling or transporter internalization compared to a wild-type form of the membrane transport protein.

For example, the mutant membrane transport protein is a mutant glucose transport (GLUT) protein having a reduced rate of recycling or transporter internalization compared to a wild-type form of the membrane transport protein.

For instance, the reduced rate of recycling or transporter internalization of the mutant membrane transport protein increases the level of the mutant membrane transport protein at the plasma membrane of a cell compared to the level of a wild-type form of the membrane transport protein.

In an example, the mutant GLUT protein is a mutant GLUT4 protein, e.g., the mutant GLUT4 protein comprises an amino acid sequence at least 80% identical to an amino acid sequence selected from the group consisting of SEQ ID NO: 6, SEQ ID NO: 8 and SEQ ID NO: 10.

For example, the membrane transport protein is labeled to facilitate binding of the ligand to the membrane transport protein.

In an example, the label comprises one or more copies of a peptide, polypeptide or protein that is heterologous to the membrane transport protein. For example, the label comprises one or more copies of a peptide, polypeptide or protein selected from the group consisting of influenza virus hemagglutinin (HA) (SEQ ID NO: 15), Simian Virus 5 (V5) (SEQ ID NO: 16), polyhistidine (SEQ ID NO: 17), c-myc (SEQ ID NO: 18), FLAG (SEQ ID NO: 19), GST (SEQ ID NO: 22), MBP (SEQ ID NO: 23), GAL4 (SEQ ID NO: 24), β-galactosidase (SEQ ID NO: 25), enhanced green fluorescence protein (eGFP) (SEQ ID NO: 26), yellow fluorescent protein (SEQ ID NO: 27), soluble modified blue fluorescent protein (SEQ ID NO: 28), soluble-modified red-shifted green fluorescent protein (SEQ ID NO: 29), cyan fluorescent protein (SEQ ID NO: 30), biotin, strepavidin, a peptide comprising the amino acid sequence set forth in SEQ ID NO: 20, a peptide comprising the amino acid sequence set forth in SEQ ID NO: 21, a peptide comprising the amino acid sequence set forth in SEQ ID NO: 31 and mixtures thereof.

In one exemplified form of the invention, the label comprises the amino acid sequence set forth in SEQ ID NO: 8.

For example, the label is positioned within an extracellular domain of the membrane transport protein, e.g., the label is positioned within the first extracellular domain of a GLUT protein or a mutant thereof.

For example, the labeled membrane transport protein is a GLLUT4 protein or a mutant GLUT4 protein that comprises an amino acid sequence at least 80% identical to an amino acid sequence selected from the group consisting of SEQ ID NO 4, SEQ ID NO: 6, SEQ ID NO: 8 and SEQ ID NO: 10.

In another example, the labeled membrane transport protein is a GLUT1 protein that comprises an amino acid sequence at least 80% identical to the amino acid sequence set forth in SEQ ID NO: 13.

In an example of the invention, the cell is a eukaryotic cell, for example, the cell is a mammalian cell, e.g., a cell selected from the group consisting of a 3T3-L1 fibroblast cell, a 3T3-L1 adipocyte cell and a C2C12 cell.

In an example, the ligand capable of binding to the membrane transport protein is an antibody. For example, the antibody is a monoclonal antibody, e.g., an anti-HA tag antibody.

For example, the antibody is labeled with a detectable marker selected from the group consisting of an enzyme label, a radiolabel and a fluorescent label, e.g., the antibody is labeled with a fluorescent label.

In an example, the plasma membrane is permeablilized or disrupted by contacting the plasma membrane with an agent that permeabilizes or disrupts a membrane for a time and under conditions sufficient for permeabilization or disruption to occur. For example, the agent that permeabilizes or disrupts a membrane is selected from the group consisting of saponin, n-octyl-glucopyranoside, n-Dodecyl β-D-maltoside, N-Dodecanoyl-N-methylglycine sodium salt, hexadecyltrimethylammonium bromide, deoxycholate, a non-ionic detergent, streptolysin-O (SEQ ID NO: 32), α-hemolysin (SEQ ID NO: 33), tetanolysin (SEQ ID NO: 34) and mixtures thereof, e.g., the agent that permeabilizes or disrupts the membrane is saponin.

In an example of the invention, the level of the ligand bound to the membrane transport protein is determined by a process comprising contacting the ligand with an antibody that specifically binds to the ligand for a time and under conditions sufficient for an antibody-antigen complex to form and determining the level of the complex wherein the level of the complex indicates the level of the ligand bound to the membrane transport protein.

For example, the level of the ligand bound to the membrane transport protein is determined using an assay selected from the group consisting of immunfluorescence, immunohistochemistry, and an immunosorbent assay, e.g., the level of the ligand bound to the membrane transport protein is determined using a fluorescence linked immunosorbent assay.

In one example, the process of the invention additionally comprises providing the cell expressing the membrane transport protein. For example, providing the cell expressing the membrane protein comprises transforming or transfecting the cell with an expression construct that encodes the membrane protein.

In an example, the process additionally comprises fixing the cell. For example, the cell is fixed prior to or at the same time as permeabilizing or disrupting the plasma membrane of the cell.

In an example, the cell is fixed with a compound selected from the group consisting of formaldehyde, paraformaldehyde, alcohol, methanol and glutaraldehyde, e.g., the cell is fixed with formaldehyde.

In another example, the present invention additionally comprises inducing translocation of the membrane transport protein to the plasma membrane. For example, inducing translocation of the membrane transport protein to the plasma membrane comprises contacting the cell with an amount of one or more peptides, polypeptides, proteins or compounds sufficient to induce translocation of the membrane transport protein for a time and under conditions sufficient for translocation to occur.

For instance the cell is contacted with sucrose and/or insulin, e.g., the cell is contacted with sucrose and/or insulin in the presence of serum.

In another example, the process additionally comprises inducing resistance to translocation of the membrane transport protein in the cell. For example, the membrane transport is a GLUT protein or a mutant GLUT protein and wherein inducing resistance to translocation of the membrane transport protein in the cell comprises contacting the cell with an amount of insulin sufficient to induce resistance to insulin induced translocation for a time and under conditions sufficient for resistance to insulin induced translocation to occur.

For example, the cell is contacted with insulin in the absence of serum, e.g., the cell is contacted with insulin for between about 24 hours and about 48 hours.

The present invention provides a process for determining the level of a membrane transport protein translocated to the plasma membrane of a cell, said process comprising:
(a) determining the level of the membrane transport protein at the plasma membrane of a cell using a method comprising:
   (i) contacting a cell with a ligand that binds to an extracellular domain of the membrane transport protein for a time and under conditions sufficient for the ligand to bind to the membrane transport protein; and
   (ii) determining the level of ligand bound to the membrane transport protein;
(b) determining the level of the membrane transport protein within another cell using a method comprising:
   (i) permeabilizng or disrupting the other cell;
   (ii) contacting the membrane transport protein within the cell with the ligand for a time and under conditions sufficient for the ligand to bind the membrane transport protein;
   (iii) determining the level of ligand bound to the membrane transport protein; and
(c) comparing the level of ligand detected at (a) (ii) and (b) (iii) to determine the level of the labeled membrane transport protein at the plasma membrane relative to the total level of labeled membrane transport protein.

For example, the cells are isogenic or from the same cell line.

For instance, the cells are cultured under substantially similar conditions.

In an example, the level of the membrane transport protein at the plasma membrane of the cell and the level of membrane transport protein within the cell are each determined in a plurality of cells.

For example, the process of the invention additionally comprises normalizing the determined level of ligand bound to the membrane transport protein with regard to the number of cells in which the level of ligand bound to the membrane transport protein is determined.

For example, the number of cells is determined by a method comprising contacting the cells with an antibody or ligand capable of binding to a cell or component thereof for a time and under conditions sufficient for binding of the antibody or ligand to the cell or component thereof and determining the level of antibody bound to the cells, wherein the level of antibody or ligand bound to the cells is indicative of the number of cells, e.g., the ligand is wheat germ agglutinin.

The present invention additionally provides a process for determining the level of a labeled GLUT4 protein or labeled mutant GLUT4 protein translocated to the plasma membrane of a cell, wherein said labeled mutant GLUT4 protein has a reduced rate of recycling or transporter internalization compared to a wild-type form of the membrane transport protein, said process comprising:
(a) determining the level of the labeled GLUT4.protein or labeled mutant GLUT4 protein at the plasma membrane of a cell expressing the labeled GLUT4 protein or labeled mutant GLUT4 protein using a method comprising:
   (i) contacting the cell with a ligand that binds to the label for a time and under conditions sufficient for the ligand to bind to the labeled GLUT4 protein or labeled mutant GLUT4 protein; and
   (ii) determining the level of ligand bound to the labeled GLUT4 protein or labeled mutant GLUT4 protein;
(b) determining the level of membrane transport protein within another cell expressing the labeled GLUT4 protein or labeled mutant GLUT4 protein using a method comprising:
   (i) permeabilizing or disrupting the other cell;
   (ii) contacting the labeled GLUT4 protein or labeled mutant GLUT4 protein within the cell with a ligand that binds to the label for a time and under conditions sufficient for the ligand to bind to the labeled GLUT4 protein or labeled mutant GLUT4 protein;
   (iii) determining the level of ligand bound to the labeled GLUT4 protein or labeled mutant GLUT4 protein; and
(c) comparing the level of ligand detected at (a) (ii) and (b) (iii) to determine the level of the labeled GLUT4 protein or labeled mutant GLUT4 protein at the plasma membrane relative to the total level of labeled GLUT4 protein or labeled mutant GLUT4 protein.

The present invention additionally provides a process for determining the level of a labeled GLUT4 protein or a labeled mutant GLUT4 protein translocated to the plasma membrane of a cell that is resistant to insulin induced GLUT4 translocation, wherein said labeled mutant GLUT4 protein has a reduced rate of recycling or transporter internalization compared to a wild-type form of the membrane transport protein, said process comprising:
(a) contacting a plurality of cells expressing a labeled GLUT4 protein or a labeled mutant GLUT4 protein with an amount of insulin sufficient to induce resistance to insulin induced translocation for a time and under conditions sufficient to induce resistance to insulin induced GLUT4 translocation in the cell, wherein the cells are contacted with insulin in the absence of serum and wherein the cells are contacted with insulin for a period of time from about 24 hours to about 48 hours;
(b) determining the level of the labeled GLUT4 protein or labeled mutant GLUT4 protein at the plasma membrane of a cell (a) using a method comprising:
   (i) contacting the cell with a ligand that binds to the label for a time and under conditions sufficient for the ligand to bind to the labeled GLUT4 protein or labeled mutant GLUT4 protein; and
   (ii) determining the level of ligand bound to the labeled GLUT4 protein or labeled mutant GLUT4 protein;
(c) determining the level of labeled GLUT4 protein or labeled mutant GLUT4 protein in another cell (a) using a method comprising:
   (i) permeabilizing or disrupting the other cell;
   (ii) contacting the labeled GLUT4 protein or labeled mutant GLUT4 protein within the cell with a ligand that binds to the label for a time and under conditions sufficient for the ligand to bind to the labeled GLUT4 protein or labeled mutant GLUT4 protein;
   (iii) determining the level of ligand bound to the labeled GLUT4 protein or labeled mutant GLUT4 protein; and
(d) comparing the level of ligand detected at (b) (ii) and (c) (iii) to determine the level of the labeled GLUT4 protein or labeled mutant GLUT4 protein at the plasma membrane relative to the total level of labeled GLUT4 protein or labeled mutant GLUT4 protein.

The present invention additionally provides a process for determining the level of recycling of a membrane transport protein in a cell comprising:
(a) determining the level of the membrane transport protein translocated to the plasma membrane of a cell using the process of the invention;
(b) determining the level of the membrane transport protein translocated to the plasma membrane of another cell using the process of the invention, wherein the other cell is cultured for a longer period of time than the cell (a); and
(c) comparing the level of the membrane transport protein translocated to the plasma membrane at (a) and (b) to determine the level of recycling of the membrane transport protein in the cell.

The present invention additionally provides a process for determining a change in the level of recycling, of a membrane transport in a cell comprising:
(a) determining the level of the membrane transport protein translocated to the plasma membrane of a cell using the process of the invention;
(b) determining the level of the membrane transport protein translocated to the plasma membrane of another cell using the process of the invention, wherein the other cell is cultured for a longer period of time than the cell (a); and
(c) comparing the level of the membrane transport protein translocated to the plasma membrane at (a) and (b),
   wherein a change in the level of the membrane transport protein translocated to the plasma membrane indicates a change in the level of recycling of a membrane transport protein.

The present invention additionally provides a process for determining a mutation in a nucleic acid encoding a mutant membrane transport protein, wherein said mutation modulates translocation of said membrane transport protein, said method comprising:
(i) determining the level of the mutant membrane transport protein translocated to the plasma membrane of a cell using the process of the invention; and
(ii) determining the level of the wild-type form of the membrane transport protein translocated to the plasma membrane of a cell using the process of the invention,
   wherein an enhanced or suppressed level of translocation of the membrane transport protein at (a) compared to (b) indicates that the nucleic acid comprises a mutation that modulates the level of level of translocation of the membrane transport protein to the plasma membrane.

The present invention additionally provides a process for determining an agent that modulates translocation of a membrane transport protein to the plasma membrane of a cell, said process comprising:
(a) determining the level of a membrane transport protein translocated to the plasma membrane of a cell in the absence of a candidate agent by performing the process of the invention;
(b) determining the level of the membrane transport protein translocated to the plasma membrane of a cell in the presence of the candidate agent by performing the process of the invention, wherein a difference in the level of the membrane transport protein translocated to the plasma membrane of a cell at (a) compared to (b) indicates that the candidate agent modulates translocation of the membrane transport protein.
(c) optionally, determining the structure of the candidate agent;
(d) optionally, providing the name or structure of the candidate agent; and
(e) optionally, providing, the candidate agent.

The present invention further provides a process for determining a candidate compound for the treatment of insulin resistance comprising:
(a) determining the level of the labeled GLUT4 protein or the labeled mutant GLUT4 protein translocated to the plasma membrane of a cell in the absence of a candidate agent by performing the process for determining the level of a labeled GLUT4 protein or a labeled mutant GLUT4 protein translocated to the plasma membrane of a cell that is resistant to insulin induced GLUT4 translocation, wherein said labeled mutant GLUT4 protein has a reduced rate of recycling or transporter internalization compared to a wild-type form of the membrane transport protein; and
(b) determining the level of the labeled GLUT4 protein or a labeled mutant GLUT4 protein translocated to the plasma membrane of another cell in the presence of the candidate agent by performing the process for determining the level of a labeled GLUT4 protein or a labeled mutant GLUT4 protein translocated to the plasma membrane of a cell that is resistant to insulin induced GLUT4 translocation, wherein said labeled mutant GLUT4 protein has a reduced rate of recycling or transporter internalization compared to a wild-type form of the membrane transport protein and wherein a candidate agent that enhances the level of translocation of the labeled GLUT4 protein or a labeled mutant GLUT4 protein is a candidate agent for the treatment of insulin resistance.
(c) optionally, determining the structure of the candidate agent;
(d) optionally, providing the name or structure of the candidate agent; and
(e) optionally, providing, the candidate agent.

For example, the insulin resistance is associated with diabetes, e.g., the diabetes is type II diabetes.

The present invention additionally provides a process for manufacturing a medicament for the treatment of insulin resistance comprising:
(a) determining a candidate compound for the treatment of insulin resistance using a process comprising:
   (i) determining the level of the labeled GLUT4 protein or the labeled mutant GLUT4 protein translocated to the plasma membrane of a cell in the absence of a candidate agent by performing the process for determining the level of a labeled GLUT4 protein or a labeled mutant GLUT4 protein translocated to the plasma membrane of a cell that is resistant to insulin induced GLUT4 translocation, wherein said labeled mutant GLUT4 protein has a reduced rate of recycling or transporter internalization compared to a wild-type form of the membrane transport protein; and
   (ii) determining the level of the labeled GLUT4 protein or a labeled mutant GLUT4 protein translocated to the plasma membrane of another cell in the presence of the candidate agent by performing the process for determining the level of a labeled GLUT4 protein or a labeled mutant GLUT4 protein translocated to the plasma membrane of a cell that is resistant to insulin induced GLUT4 translocation, wherein said labeled mutant GLUT4 protein has a reduced rate of recycling or transporter internalization compared to a wild-type form of the membrane transport protein and wherein a candidate agent that enhances the level of translocation of the labeled GLUT4 protein or a labeled mutant GLUT4 protein is a candidate agent for the treatment of insulin resistance.
(b) optionally, isolating the candidate agent;
(c) optionally, providing the name or structure of the candidate agent;
(d) optionally, providing the candidate agent; and
(e) using the candidate agent in the manufacture of a medicament for the treatment of insulin resistance.

### BRIEF DESCRIPTION OF THE FIGURES

FIG. 1A is a schematic representation of a recombinant GLUT4 protein that is labeled with a HA epitope. Note that when expressed in a cell the HA epitope is within the first extracellular domain of the protein. This location of the HA epitope facilitates detection of the GLUT4 protein when translocated to the plasma membrane without disrupting said plasma membrane.

FIG. 1B is a schematic representation showing the various forms of GLUT4 used in the analysis of translocation of GLUT4 to the plasma membrane. WT represents the wild-type form of GLUT4 (SEQ ID NO: 1) TAIL represents a mutant form of GLUT4 in which the residues at the C-terminus of GLUT4 have been mutated (SEQ ID NO: 5); L489,490A represents a mutant form of GLUT4 in which a di-leucine motif at the C-terminal end of GLUT4 has been mutated to a di-Alanine motif (SEQ ID NO: 6); and F5A represents a mutant form of GLUT4 in which the phenylalanine at amino acid number 5 of GLUT4 has been mutated to Alanine (SEQ ID NO: 7), wherein each of these proteins have been labeled with a HA epitope tag (SEQ ID NO: 18) in an intracellular domain, for example, the sequence of a WT, GLUT4 labeled with an HA epitope tag is represented by SEQ ID NO: 3.

FIG. 1C is a schematic representation of one example of the method of detecting the amount of GLUT4 that has translocated to the plasma membrane. The left hand side of the figure shows a cell that is stained to determine the amount of GLUT4 that has translocated to the membrane. Recombinant GLUT4 labeled with a HA epitope is expressed in the cell; the cell is then fixed and the GLUT4 that has translocated to the plasma membrane is detected with an anti-HA antibody; the cell is then permeablilized with saponin and the anti-HA antibody detected with a fluorescent secondary antibody. The right hand side of the figure shows a cell that is used to determine the total amount of GLUT4 in a cell. Recombinant GLUT4 labeled with a HA epitope is expressed in the cell; the cell is then fixed; and permeablilized with saponin. The HA epitope is then detected with an anti-HA antibody, which is now able to enter the cell. The anti-HA epitope is then detected with a fluorescent secondary antibody. Comparing the results obtained from the two cells shows the amount of GLUT4 that has translocated to the plasma membrane as a function of total GLUT4.

FIG. 1D is a copy of a photographic representation showing 3T3-L1 adipocytes expressing HA-GLUT4 WT immunolabeled with an anti-HA or anti-GLUT4 for the detection of HA-GLUT4 or total GLUT4 content respectively.

FIG. 1E is a copy of a photographic representation showing an immunoblot on which cell extracts from 3T3-L1 fibroblasts (F) or 3T3-L1 adipocytes (A) expressing the indicated HA-tagged GLUT4 protein were analyzed using the indicated antibody (left hand side).

FIG. 1F is a graphical representation showing the level of expression of each of the HA-tagged GLUT4 proteins shown in FIG. 1C

FIG. 1G is a copy of a photographic representation of various cells used to analyze the translocation of GLUT4. The top row of cells are 3T3-L1 fibroblasts and the bottom row 3T3-L1 adipocytes. From left to right the cells were not transduced (i.e. do not express a tagged GLUT4); were transduced with a tagged WT, GLUT4; were transduced with a tagged TAIL mutant GLUT4; were transduced with a tagged L489,490A mutant GLUT4; or were transduced with a tagged F5A mutant GLUT4.

FIG. 2A is a graphical representation of the effect of insulin that do not express HA-tagged GLUT4. The amount of fluorescence detected using the anti-HA antibody (HA) was the same as that detected with a non-relevant (NR) antibody, indicating that the anti-HA antibody does not non-specifically bind a protein in the cell.

FIG. 2B is a graphical representation of the amount of HA tagged GLUT4 detected at the plasma membrane of 3T3-L1 adipocytes incubated in the presence of 200 nM insulin. Over time, the amount of HA-tagged GLUT4 (squares) detected at the plasma membrane increased, while the amount of the non-relevant protein (triangles) remained constant. This indicates that insulin induces GLUT4 translocation to the plasma membrane.

FIG. 2C is a graphical representation of the percentage of total GLUT4 in a cell that has translocated the plasma membrane in the presence of 200 nM insulin. Using the method described herein the amount of HA tagged GLUT4 that was translocated to the plasma membrane in the presence of insulin was determined relative to the total HA-tagged GLUT4 in a cell.

FIG. 2D is a graphical representation of the percentage of total GLUT4 in a cell that has translocated to the plasma membrane in the presence of various concentrations of insulin. Using the method described herein the effect of insulin concentration on the amount of HA-tagged GLUT4 translocation to the plasma membrane relative to the total HA-tagged GLUT4 was determined (triangle). In the presence of wortmanin (squares) insulin induced translocation of GLUT4 was almost totally abrogated.

FIG. 3A is a graphical representation showing the amount of a HA-tagged form of GLUT4 (from left to right: WT; TAIL; L489; 490A; and F5A) detected at the plasma membrane of 3T3-L1 fibroblasts at relative to the total HA-tagged form of GLUT4. Clearly GLUT4 translocation is induced by insulin in fibroblasts.

FIG. 3B is a graphical representation showing the percentage of a HA-tagged form of GLUT4 (from left to right: WT; TAIL; L489; 490A; and F5A) at the plasma membrane of 3T3-L1 adipocytes in the presence of 200 nM insulin. Interestingly, the L489; L490A and F5A mutants, which are believed to be impaired in their internalization/recycling, show an increase in adipocytes compared with fibroblasts (FIG. 3A).

FIG. 4 is a graphical representation showing the internalization kinetics of HA-GLUT4 in 3T3-L1 adipocytes. Adipocytes expressing the indicated GLUT4 molecule were incubated for 20 min with 200 nM insulin at 37° C. and for 1 h with anti-HA antibody on ice. Excess antibody was washed away, and cells were incubated for the indicated periods at 37° C. in the presence of either 100 nM wortmanin, to measure GLUT4 internalization in the basal state, or 200 nM insulin. Cells were exposed to fixative and incubated with fluorescent secondary antibody in the absence of permeabilg agent to allow measurement of the time-dependent disappearance of anti-HA-labeled GLUT4 from the cell surface.

FIG. 5A is a copy of a photographic representation showing the subcellular localization of HA-tagged GLUT4 in adipocytes incubated for 2 hours with 200 nM insulin and subsequently for 2 hours without insulin and then 20 minutes without insulin.

FIG. 5B is a copy of a photographic representation showing the subcellular localization of HA-tagged GLUT4 in adipocytes incubated for 2 hours with 200 nM insulin and subsequently for 2 hours with insulin and then 20 minutes without insulin.

FIG. 5C is a copy of a photographic representation showing the subcellular localization of HA-tagged GLUT4 in adipocytes incubated for 2 hours with 200 nM insulin and anti-HA antibody and subsequently for 2 hours without insulin and anti-HA antibody and then 20 minutes without insulin.

FIG. 5D is a copy of a photographic representation showing the subcellular localization of HA-tagged GLUT4 in adipocytes incubated for 2 hours with 200 nM insulin and anti-HA antibody and subsequently for 2 hours without insulin and anti-HA antibody and then 20 minutes with insulin.

FIG. 5E shows graphical representations showing levels of antibody uptake in fibroblasts or adipocytes as indicated at the left hand-side of the figure expressing the indicated HA-GLUT4 protein. Cells were incubated with (squares) or without (triangles) 200 nM insulin for 20 min, after which anti-HA antibody was added. Cells were incubated for up to 180 minutes, fixed permeablilized and incubated with a fluorescently labeled secondary antibody. The level of anti-HA antibody taken up by the cells is expressed as a percentage of total post-fixation anti-HA labeling.

FIG. 6A is a graphical representation demonstrating the existence of a non-recycling pool of HA-GLUT4 WT in a cell. Cells were incubated in the presence of insulin for an extended period of time (180 min) and the level of HA-GLUT4 at the plasma membrane relative to the total level detected in the cell was determined.

FIG. 6B is a graphical representation showing the level of HA-GLUT4 in the cells used to determine the level of HA-GLUT4 in the cell (FIG. 6A) following an additional incubation with fixative.

FIG. 6C is a graphical representation showing the level of HA-GLUT4 detected at the plasma membrane of cells in which the level of HA-GLUT4 at the plasma membrane was previously determined (FIG. 6A) following an additional incubation with an anti-HA antibody (and detection of the level of bound anti-HA antibody).

FIG. 6D is a graphical representation showing the level of of HA-GLUT4 detected within cells previously fixed and permeablilized following an additional incubation with an anti-HA antibody (and detection of the level of bound anti-HA antibody).

FIG. 6E is a graphical representation showing the relative level (percentage of total) level of HA-GLUT4 WT detected at the plasma membrane of a cell using various concentrations of anti-HA antibody.

FIG. 6F is a graphical representation showing the relative level (percentage of total) of HA-GLUT4 WT detected at the plasma membrane of a cell following a 2 hour incubation in the presence of cycloheximide.

FIG. 6G is a graphical representation showing the effect of endosomal pH on the binding of the anti-HA antibody to HA-GLUT4. Cells were incubated for 30 min at 37□C in hypertonic medium (0.45 M sucrose, pH 7.4), on ice with antibody in the same medium, and at 37□C in hypertonic buffer at pH 7.4 or pH 5.5 in the absence of antibody. Release of antibody from the PM at neutral or endosomal pH was determined by incubating fixed non-permeablilized cells with fluorescent secondary antibody.

FIG. 6H is a graphical representation showing the effect of incubating a cell in the presence of insulin for an extended period of time. Cells were incubated in the presence of 200 nM insulin for up to 3 hours and the relative level (percentage of total) of HA-GLUT4 at the plasma membrane determined.

FIG. 7 shows graphical and photographic representations showing GLUT4 recycling during the differentiation of 3T3-L1 fibroblasts into adipocytes. FIG. 5. Cells were analyzed at different stages during differentiation as indicated. After incubation for 18 h in medium containing fetal bovine serum and for 2 h in the absence of serum, the cells were incubated in the continuous presence of anti-HA antibody as described for FIG. 4. Parallel cultures were incubated similarly but analyzed by immunofluorescence confocal microscopy Deft microscopy panels). Non-infected cells were analyzed for endogenous GLUT4 and lipid droplet content during differentiation (right microscopy panels). Bottom right microscopy panels show Z section image of the cells. White dotted lines mark the contours of the cells.

FIG. 8A is a graphical representation showing a correlation between insulin concentration and the size of the non-recycling GLUT4 pool in 3T3-L1 adipocytes. 3T3-L1 adipocytes expressing HA-GLUT WT or HA-GLUT TRAIL were incubated at 37° C. with anti-HA antibody and the indicated concentration of insulin and the level of cell associated HA antibody was determined.

FIG. 8B is a graphical representation showing 3T3-L1 adipocytes expressing HA-GLUT4 WT or HA-GLUT4 TAIL that were incubated for 20 min at 37° C. with 0.032, 0.24, 3.2, 15 or 200 nM insulin and amounts of GLUT4 at the PM were determined and expressed as percentage of maximal insulin-induced GLUT4 translocation.

FIG. 8C is a copy of a photographic representation showing HA-GLUT4-expressing 3T3-L1 adipocytes incubated for 3 h with anti-HA antibody and the indicated concentrations of insulin. Cells were fixed, permeablilized, incubated with fluorescent secondary antibody and analyzed by confocal immunofluorescence microscopy.

FIG. 9 is a graphical representation showing the translocation of HA-GLUT4 in 3T3-L1 adipocytes grown and differentiated in a 384-well plate compared to cells grown and differentiated in a Petri dish and transferred to a 384-well plate. Axes are time of insulin exposure (min, X-axis) and percentage of total HA-GLUT4 detected at the plasma membrane (Y-axis).

FIG. 10 is a graphical representation showing the effect of amino acid concentration on the level of HA-GLUT4 translocated to the plasma membrane of a cell. HA-GLUT4 expressing adipocytes were serum starved for 2 hours in Krebs Ringer Phosphate buffer or in the same buffer supplemented with amino acid concentrations used in Dulbecco's modified eagle medium of Gibco (2× amino acids) or with half of the amino acid concentration (1× amino acids) as indicated. Axes are time of insulin exposure (min, X-axis) and percentage of total HA-GLUT4 detected at the plasma membrane (Y-axis).

FIG. 11 is a graphical representation showing the effect of insulin and sucrose on HA-GLUT4 translocation. 3T3-L1 adipocytes expressing HA-GLUT4 WT were serum starved for 2 hours at 37° C. Following 20 minutes of acute insulin stimulation with 200 nM, cells were incubated for additional 2 hours in serum free medium supplemented with 0.2% BSA and 0.3 or 0.6M sucrose as indicated. After post-fixation anti-HA immunolabeling the amount of cell surface HA-GLUT4 levels was determined. Axes are insulin concentration (nM, X-axis) and percentage of total HA-GLUT4 detected at the plasma membrane (Y-axis).

FIG. 12A is a graphical representation showing the induction of insulin resistance in 3T3-L1 adipocytes. 3T3-L1 adipocytes retrovirally infected with HA-GLUT4 were incubated 24 hours or 48 hours either with 600 nM insulin or with medium alone. After this chronic insulin stimulation for the indicated periods of time, cells were washed and 200 nM insulin added for additional 10 or 30 minutes and cell surface levels of HA-GLUT4 were measured using the fluorescence based assay. Treatment groups are indicated. Y axis shows the percentage of total HA-GLUT4 detected at the plasma membrane.

FIG. 12B is a graphical representation showing the induction of insulin resistance in 3T3-L1 adipocytes expressing a mutant GLUT4. 3T3-L1 adipocytes retrovirally infected with HA-GLUT4 TAIL mutant were incubated 24 hours or 48 hours either with 600 nM insulin or with medium alone. After this chronic insulin stimulation for the indicated periods of time, cells were washed and 200 nM insulin added for additional 10 or 30 minutes and cell surface levels of HA-GLUT4 TAIL were measured using the fluorescence based assay. Treatment groups are indicated. Y axis shows the percentage of total HA-GLUT4 detected at the plasma membrane.

FIG. 13 is a graphical representation showing the effect of wortmanin on acute and chronic insulin induced GLUT4 translocation. HA-GLUT4 expressing 3T3-L1 adipocytes were grown in 96 well plates, incubated for 2 hours or overnight in medium supplemented with 10% fetal calf serum or no serum. 200 nM insulin in case of acute stimulation and 600 nM insulin in case of chronic stimulation were used (as indicated). Following overnight stimulation cells were washed and 200 nM fresh insulin was added for 10 or 30 min. Both medium conditions were tested in the presence and absence of 100 nM wortmanin. Y axis shows the percentage of total HA-GLUT4 detected at the plasma membrane.

### DETAILED DESCRIPTION OF THE PREFERRED EMBODIMENTS

The present invention provides a process for determining the level of a membrane transport protein translocated to the plasma membrane of a cell, said method comprising:
(a) determining the level of a membrane transport protein at the plasma membrane using a method comprising:
   (i) contacting the membrane transport protein with a ligand that binds to an extracellular domain of the membrane transport protein for a time and under conditions sufficient for the ligand to bind to the membrane transport protein; and
   (ii) determining the level of ligand bound to the membrane transport protein;
(b) (i) permeabilizing or disrupting the plasma membrane of a cell and contacting the membrane transport protein within the cell with a ligand that binds to an extracellular domain of the membrane transport protein for a time and under conditions sufficient for the ligand to bind to the membrane transport protein; and
(ii) determining the level of ligand bound to the membrane transport protein within the cell; and
(c) comparing the level of ligand detected at (a) (ii) and (b) (ii) to determine the level of the membrane transport protein at the plasma membrane relative to the level of the membrane transport protein inside the cell.

For example, a ligand of a membrane transport protein that binds to an extracellular domain of the membrane transport protein is, for example, an antibody. Antibodies that bind an extracellular domain of a membrane protein are known in the art. For example, monoclonal antibody mAb5 or mAb263 that specifically bind an extracellular region of the growth hormone receptor protein (available from AGEN Limited, Acacia Ridge, Queensland, Australia). A polyclonal antibody that bind to an extracellular domain of GLUT2 is available from Alpha Diagnostics International Inc., San Antonio, Tex., USA. An antibody that binds to an extracellular domain of GLUT1 is described in Carbó et al., Clinical and Experimental Pharmacology and Physiology 30: 64, 2003. Alternatively, the antibody or ligand is produced by a method known in the art and/or described herein.

Membrane Transport Proteins

As used herein, the term “membrane transport protein” shall be taken to mean a peptide, polypeptide or protein that catalyzes the movement of a molecule across a membrane, whether this movement is by diffusion (simple or facilitated) or active transport. Membrane transport proteins in the present context exist as intracellular proteins and are capable of being membrane-localized. Such a protein may be, for example, a channel, a transporter, an ATP pump, a symporter or an antiporter. The term “membrane transport protein” shall be taken to include mutant forms of a membrane transport protein (for example, a mutant form of a membrane transport protein capable of translocating to the plasma membrane of a cell) and/or a labeled membrane transport protein. For example, a labeled membrane transport protein described herein.

For example, a membrane transport protein useful in performance of the invention is a protein from a family of proteins selected from the group consisting of amino acid/auxin permease (AAAP) family, amino acid-polyamine-organocationi (APC) family, cation-chloride cotransporter (CCC) family, hydroxy/aromatic amino acid permease (HAAAP) family, bile acid:NA⁺ symporter (BASS) family, arsenical resistance-3 (ARC3) family, monovalent cation:protein antiporter-l (CPA1) family, monovalent cation:proton antiporter-2 (CPA2) family, Na⁺-transporting carboxylic acid decarboxylase (NaT-DC) family, citrate-Mg²⁺:H⁺ (MitM) citrate-Ca²⁺:H⁺ (CitH) symporter (CitMHS) family, C₄-dicarboxylate uptake (Dcu) family, lactate permease (LctP) family, NhaB Na⁺:H⁺ antiporter (NhaB) family, NhaC Na⁺:H⁺ antiporter (NhaC) family, arsenite-antimonite (ArsB) efflux family, divalent anion:Na⁺ symporter (DASS) family, tripartite ATP-independent periplasmic transporter (IRAP-T) family, C₄-dicarboxylate uptake C (DcuC) family, NhaD Na⁺:H⁺ antiporter (NhaD) family, p-aminobenzyol-glutamate transporter (AbgT) family, gluconate:H⁺ symporter (GntP) family, L-lysine exporter (LysE) family, major facilitator superfamily (MFS), proton-dependent oligopeptide transporter (POT) family, organo-anion transporter (OAT) family, folate-biopterin transporter (FBT) family, PTS galactilol (Gat) family, PTS L-ascorbate (L-Asc) family, PTS glucose-glucoside (Glc) family, PTS fructose-mannitol (Fru) family, voltage-gated ion channel (VIC) family, glutamate gated ion channel (GIC) family of neurotransmitter receptors, animal inward rectifier K⁺ channel (RIR-CaC) family, ryanodine-inositol 1,4,5-triphosphate receptor Ca²⁺ channel (RIR-CaC) family and K⁺ transporter (Trk) family. Information concerning the structure and/or function of a membrane transport protein (e.g., a membrane transport protein from a family described supra) is found in, for example, the Transport Classification Database available from University of California, San Diego, La Jolla, Calif., USA.

For example, the membrane transport protein is a human membrane transport protein. For example, a human membrane transport protein selected from the group consisting of a human annexin, a human ATP-binding cassette transporter, a human ATPase, a human calcium channel, a human potassium channel, a human sodium channel and a human solute carrier.

For example, the membrane transport protein is a protein that translocates to a plasma membrane of a cell under normal physiological conditions, or following stimulation by a condition or agent, such as, for example, glucose or insulin. Preferably the membrane transport protein is, for example, an ABC transporter protein, a P class ATP pump, a F class ATP pump, a V class ATP pump, a Cl⁻ channel, a H⁺ channel and Ca⁺⁺ channel, a K⁺ channel, an uniporter a symporter or an antiporter. For example, the membrane transport protein is a membrane transport protein selected from the group consisting of ABC1, ABCA2, ABCA3, ABCR, ABCA5, ABCA6, ABCA7, ABCA8, ABCA9, ABCA10, ABCA12, ABCA13, PGY1, TAP1, TAP2, PGY3, ABCB5, ABCB6, ABC7, M-ABC1, ABCB9, ABCB10, BSEP, MRP1, MRP2, MRP3, MRP4, MRP5, MRP6, CFTR, SUR1, SUR2, ABCC10, ABCC11, ABCC12, ABCC13, ALD, ALDL1, ABCD2, PXMP1, PXMP1L, RNASEL1, ABC50, ABCF2, ABCF3, ABCG1, ABCG2, ABCG4, ABCG5, ABCG8, KCNA1, CACNL1A4, KCNQ2, KCNQ3, SCN1B, CHRNA4, GLRA1, KCNE1, KCNQ4, SCN4A, CACNL1A3, CLCN1, CNCN1, RYR1, CHRNA1, KCNQ1, HERG, SCN5A, KCNE1, SCN5A, KCNE1, GLUT1, GLUT2, GLUT3, GLUT4, GLUT5, GLUT6, GLUT7, GLUT8, GLUT9, GLUT10, GLUT11, GLUT12, HMIT and GLUT14.

As used herein, the nomenclature for GLUT proteins and HMIT is described by Joost et al, 2001, Am. J. Physiol. Endocrinol. Metab. 282: E974-E976, 2002.

In an example of the invention, the membrane transport protein is a glucose transport protein or a facilitated glucose transport protein (GLUT). As used herein the term “glucose transport protein” or “facilitated glucose transport protein” or “GLUT” shall be taken to mean a member of the SCLC2A family of solute carrier proteins. Individual member of this family have similar predicted secondary structures with 12 transmembrane domains. Both N and C-termini are predicted to be cytoplasmic. There is a large extracellular domain between transmembrane region 1 and transmembrane region 2 and a large cytoplasmic domain between transmembrane region 6 and transmembrane region 7.

GLUT isoforms differ in their tissue expression, substrate specificity and kinetic characteristics. Table 1 outlines many of the characteristics of GLUT isoforms.

TABLE 1
GLUT isoforms

| GLUT | |
|---|---|
| Isoform | Characteristics |
| GLUT1 | mediates glucose transport into red cells, and throughout |
| | the blood brain barrier. It is ubiquitously expressed and |
| | transports glucose in most cells |
| GLUT2 | provides glucose to the liver and pancreatic cells |
| GLUT3 | the main glucose transporter in neurons |
| GLUT4 | primarily expressed in muscle and adipose tissue and |
| | regulated by insulin |
| GLUT5 | transports fructose in intestine and testis |
| GLUT6 | highly expressed in brain, spleen, and leukocytes. |
| GLUT8 | High levels are found in adult testis and placenta |
| GLUT9 | expressed in kidney, liver, placenta, lung, blood leukocytes, |
| | heart, and skeletal muscle |
| GLUT10 | widely expressed with highest levels in liver and pancreas |
| GLUT11 | expressed in heart and skeletal muscle |
| GLUT12 | expressed in skeletal muscle, adipose tissue, and small |
| | intestine |
| GLUT13 | (aka. H+ myo-inositol transporter, HMIT) predominantly |
| | expressed in brain |

For example, the process of the invention is performed with a GLUT protein selected from the group consisting of a GLUT1 protein, a GLUT2 protein, a GLUT3 protein, a GLUT4 protein, a GLUT5 protein, a GLUT6 protein, a GLUT7 protein, a GLUT8 protein, a GLUT9 protein, a GLUT10 protein, a GLUT11 protein, a GLUT12 protein, a GLUT13 (AMI) protein, a GLUT14 protein.

As used herein, the term “GLUT1 protein” shall be taken to mean a protein that comprises an amino acid sequence at least about 80% identical to the sequence set forth in SEQ ID NO: 12. For example, the protein comprises an amino acid sequence at least about 85% or at least about 90% or at least about 95% or at least about 98% or at least about 99% identical to the amino acid sequence set forth in SEQ ID NO: 12.

In one example, the GLUT1 protein is a human GLUT1 protein.

Alternatively, or in addition, a GLUT 1 protein is a protein encoded by a nucleic acid that comprises a nucleotide sequence at least about 80% identical to the nucleotide sequence set forth in SEQ ID NO: 11. For example, the nucleic acid comprises a nucleotide sequence at least about 85% or at least about 90% or at least about 95% or at least about 98% or at least about 99% identical to the nucleotide sequence set forth in SEQ ID NO: 11.

As used herein, the term “GLUT2 protein” shall be taken to mean a protein that comprises an amino acid sequence at least about 80% identical to the sequence set forth in SEQ ID NO: 38. For example, the protein comprises an amino acid sequence at least about 85% or at least about 90% or at least about 95% or at least about 98% or at least about 99% identical to the amino acid sequence set forth in SEQ ID NO: 38.

In one example, the GLU72 protein is a human GLUT2 protein.

Alternatively, or in addition, a GLUT2 protein is a protein encoded by a nucleic acid that comprises a nucleotide sequence at least about 80% identical to the nucleotide sequence set forth in SEQ ID NO: 37. For example, the nucleic acid comprises a nucleotide sequence at least about 85% or at least about 90% or at least about 95% or at least about 98% or at least about 99% identical to the nucleotide sequence set forth in SEQ ID NO: 37.

As used herein, the term “GLUT3 protein” shall be taken to mean a protein that comprises an amino acid sequence at least about 80% identical to the sequence set forth in SEQ ID NO: 40. For example, the protein comprises an amino acid sequence at least about 85% or at least about 90% or at least about 95% or at least about 98% or at least about 99% identical to the amino acid sequence set forth in SEQ ID NO: 40.

In one example, the GLUT3 protein is a human GLUT3 protein.

Alternatively, or in addition, a GLUT3 protein is a protein encoded by a nucleic acid that comprises a nucleotide sequence at least about 80% identical to the nucleotide sequence set forth in SEQ ID NO: 39. For example, the nucleic acid comprises a nucleotide sequence at least about 85% or at least about 90% or at least about 95% or at least about 98% or at least about 99% identical to the nucleotide sequence set forth in SEQ ID NO: 39.

As used herein, the term “GLUT4 protein” shall be taken to mean a protein that comprises an amino acid sequence at least about 80% identical to the sequence set forth in SEQ ID NO: 2. For example, the protein comprises an amino acid sequence at least about 85% or at least about 90% or at least about 95% or at least about 98% or at least about 99% identical to the amino acid sequence set forth in SEQ ID NO: 2.

In one example, the GLUT4 protein is a human GLUT4 protein.

Alternatively, or in addition, a GLUT 4 protein is a protein encoded by a nucleic acid that comprises a nucleotide sequence at least about 80% identical to the nucleotide sequence set forth in SEQ ID NO: 1. For example, the nucleic acid comprises a nucleotide sequence at least about 85% or at least about 90% or at least about 95% or at least about 98% or at least about 99% identical to the nucleotide sequence set forth in SEQ ID NO: 1.

As used herein, the term “GLUT5 protein” shall be taken to mean a protein that comprises an amino acid sequence at least about 80% identical to the sequence set forth in SEQ ID NO: 2. For example, the protein comprises an amino acid sequence at least about 85% or at least about 90% or at least about 95% or at least about 98% or at least about 99% identical to the amino acid sequence set forth in SEQ ID NO: 42.

In one example, the GLUT5 protein is a human GLUT5 protein.

Alternatively, or in addition, a GLUT5 protein is a protein encoded by a nucleic acid that comprises a nucleotide sequence at least about 80% identical to the nucleotide sequence set forth in SEQ ID NO: 41. For example, the nucleic acid comprises a nucleotide sequence at least about 85% or at least about 90% or at least about 95% or at least about 98% or at least about 99% identical to the nucleotide sequence set forth in SEQ D NO: 41.

As used herein, the term “GLUT6 protein” shall be taken to mean a protein that comprises an amino acid sequence at least about 80% identical to the sequence set forth in SEQ ID NO: 44. For example, the protein comprises an amino acid sequence at least about 85% or at least about 90% or at least about 95% or at least about 98% or at least about 99% identical to the amino acid sequence set forth in SEQ ID NO: 44.

In one example, the GLUT6 protein is a human GLUT6 protein.

Alternatively, or in addition, a GLUT6 protein is a protein encoded by a nucleic acid that comprises a nucleotide sequence at least about 80% identical to the nucleotide sequence set forth in SEQ ID NO: 43. For example, the nucleic acid comprises a nucleotide sequence at least about 85% or at least about 90% or at least about 95% or at least about 98% or at least about 99% identical to the nucleotide sequence set forth in SEQ ID NO: 43.

As used herein, the term “GLUT7 protein” shall be taken to mean a protein that comprises an amino acid sequence at least about 80% identical to the sequence set forth in SEQ ID NO: 46. For example, the protein comprises an amino acid sequence at least about 85% or at least about 90%/o or at least about 95% or at least about 98% or at least about 99% identical to the amino acid sequence set forth in SEQ ID NO: 46.

In one example, the GLUT7 protein is a human GLUT7 protein.

Alternatively, or in addition, a GLUT7 protein is a protein encoded by a nucleic acid that comprises a nucleotide sequence at least about 80% identical to the nucleotide sequence set forth in SEQ ID NO: 45. For example, the nucleic acid comprises a nucleotide sequence at least about 85%.or at least about 90% or at least about 95% or at least about 98% or at least about 99% identical to the nucleotide sequence set forth in SEQ ID NO: 45.

As used herein, the term “GLUT8 protein” shall be taken to mean a protein that comprises an amino acid sequence at least about 80% identical to the sequence set forth in SEQ ID NO: 48. For example, the protein comprises an amino acid sequence at least about 85% or at least about 90% or at least about 95% or at least about 98% or at least about 99% identical to the amino acid sequence set forth in SEQ ID NO: 48.

In one example, the GLUT8 protein is a human GLUT“8 protein.

Alternatively, or in addition, a GLUT8 protein is a protein encoded by a nucleic acid that comprises a nucleotide sequence at least about 80% identical to the nucleotide sequence set forth in SEQ ID NO: 47. For example, the nucleic acid comprises a nucleotide sequence at least about 85% or at least about 90% or at least about 95% or at least about 98% or at least about 99% identical to the nucleotide sequence set forth in SEQ ID NO: 4.

As used herein, the term “GLUT9 protein” shall be taken to mean a protein that comprises an amino acid sequence at least about 80% identical to the sequence set forth in SEQ ID NO: 50. For example, the protein comprises an amino acid sequence at least about 85% or at least about 90% or at least about 95% or at least about 98% or at least about 99% identical to the amino acid sequence set forth in SEQ ID NO: 50.

In one example, the GLUT9 protein is a human GLUT9 protein.

Alternatively, or in addition, a GLUT9 protein is a protein encoded by a nucleic acid that comprises a nucleotide sequence at least about 80% identical to the nucleotide sequence set forth in SEQ ID NO: 49. For example, the nucleic acid comprises a nucleotide sequence at least about 85% or at least about 90% or at least about 95% or at least about 98% or at least about 99% identical to the nucleotide sequence set forth in SEQ ID NO: 49.

As used herein, the term “GLUT10 protein” shall be taken to mean a protein that comprises an amino acid sequence at least about 80% identical to the sequence set forth in SEQ ID NO: 52. For example, the protein comprises an amino acid sequence at least about 85% or at least about 90% or at least about 95% or at least about 98% or at least about 99% identical to the amino acid sequence set forth in SEQ ID NO: 52.

In one example, the GLUT10 protein is a human GLUT10 protein.

Alternatively, or in addition, a GLUT10 protein is a protein encoded by a nucleic acid that comprises a nucleotide sequence at least about 80% identical to the nucleotide sequence set forth in SEQ ID NO: 51. For example, the nucleic acid comprises a nucleotide sequence at least about 85% or at least about 90% or at least about 95% or at least about 98% or at least about 99% identical to the nucleotide sequence set forth in SEQ ID NO: 51.

As used herein, the term “GLUT11 protein” shall be taken to mean a protein that comprises an amino acid sequence at least about 80% identical to the sequence set forth in SEQ ID NO: 54. For example, the protein comprises an amino acid sequence at least about 85% or at least about 90% or at least about 95%. or at least about 98% or at least about 99% identical to the amino acid sequence set forth in SEQ ID NO: 54.

In one example, the GLUT11 protein is a human GLUT11 protein.

Alternatively, or in addition, a GLUT11 protein is a protein encoded by a nucleic acid that comprises a nucleotide sequence at least about 80% identical to the nucleotide sequence set forth in SEQ ID NO: 53. For example, the nucleic acid comprises a nucleotide sequence at least about 85% or at least about 90% or at least about 95% or at least about 98% or at least about 99% identical to the nucleotide sequence set forth in SEQ ID NO: 53.

As used herein, the term “GLUT12 protein” shall be taken to mean a protein that comprises an amino acid sequence at least about 80% identical to the sequence set forth in SEQ ID NO: 56. For example, the protein comprises an amino acid sequence at least about 85% or at least about 90% or at least about 95% or at least about 98% or at least about 99% identical to the amino acid sequence set forth in SEQ ID NO: 56.

In one example, the GLUT12 protein is a human GLUT12 protein.

Alternatively, or in addition, a GLUT12 protein is a protein encoded by a nucleic acid that comprises a nucleotide sequence at least about 80% identical to the nucleotide sequence set forth in SEQ ID NO: 55. For example, the nucleic acid comprises a nucleotide sequence at least about 85% or at least about 90% or at least about 95% or at least about 98% or at least about 99% identical to the nucleotide sequence set forth in SEQ ID NO: 55.

As used herein, the term “GLUT13 protein” or “HMIT” shall be taken to mean a protein that comprises an amino acid sequence at least about 80% identical to the sequence set forth in SEQ ID NO: 57. For example, the protein comprises an amino acid sequence at least about 85% or at least about 90% or at least about-95% or at least about 98% or at least about 99% identical to the amino acid sequence set forth in SEQ ID NO: 57.

In one example, the GLUT13 or HMIT protein is a human GLUT13 or HMIT protein.

Alternatively, or in addition, a GLUT13 or HMIT protein is a protein encoded by a nucleic acid that comprises a nucleotide sequence at least about 80% identical to the nucleotide sequence set forth in SEQ ID NO: 56. For example, the nucleic acid comprises a nucleotide sequence at least about 85% or at least about 90% or at least about 95% or at least about 98% or at least about 99% identical to the nucleotide sequence set forth in SEQ ID NO: 56.

As used herein, the term “GLUT14 protein” shall be taken to mean a protein that comprises an amino acid sequence at least about 80% identical to the sequence set forth in SEQ ID NO: 59. For example, the protein comprises an amino acid sequence at least about 85% or at least about 90% or at least about 95% or at least about 98% or at least about 99% identical to the amino acid sequence set forth in SEQ ID NO: 59.

In one example, the GLUT14 protein is a human GLUT14 protein.

Alternatively, or in addition, a GLUT1 4 protein is a protein encoded by a nucleic acid that comprises a nucleotide sequence at least about 80% identical to the nucleotide sequence set forth in SEQ ID NO: 58. For example, the nucleic acid comprises a nucleotide sequence at least about 85% or at least about 90% or at least about 95% or at least about 98% or at least about 99% identical to the nucleotide sequence set forth in SEQ ID NO: 58.

In an exemplified form of the invention, the membrane transport protein is a GLUT4 transport protein or a GLUT1 transport protein.

In determining whether or not two amino acid sequences fall within the defined percentage identity limits supra, those skilled in the art will be aware that it is possible to conduct a side-by-side comparison of the amino acid sequences. In such comparisons or alignments, differences will arise in the positioning of non-identical residues depending upon the algorithm used to perform the alignment. In the present context, references to percentage identities and similarities between two or more amino acid sequences shall be taken to refer to the number of identical and similar residues respectively, between said sequences as determined using any standard algorithm known to those skilled in the art. In particular, amino acid identities and similarities are calculated using software of the Computer Genetics Group, Inc., University Research Park, Maddison, Wis., USA, e.g., using the GAP program of Devereaux et al., Nucl. Acids Res. 12, 387-395, 1984, which utilizes the algorithm of Needleman and Wunsch, J. Mol. Biol. 48, 443-453, 1970. Alternatively, the CLUSTAL W algorithm of Thompson et al., Nucl. Acids Res. 22, 4673-4680, 1994, is used to obtain an alignment of multiple sequences, wherein it is necessary or desirable to maximize the number of identical similar residues and to minimize the number and/or length of sequence gaps in the alignment.

Alternatively, a suite of commonly used and freely available sequence comparison algorithms is provided by the National Center for Biotechnology Information (NCBI) Basic Local Alignment Search Tool (BLAST) (Altschul et al. J. Mol. Biol. 215: 403-410, 1990), which is available from several sources, including the NCBI, Bethesda, Md. The BLAST software suite includes various sequence analysis programs including “blastn,” that is used to align a known nucleotide sequence with other polynucleotide sequences from a variety of databases and “blastp” used to align a known amino acid sequence with one or more sequences from one or more databases. Also available is a tool called “BLAST 2 Sequences” that is used for direct pairwise comparison of two nucleotide sequences.

As used herein the term “NCBI” shall be taken to mean the database of the National Center for Biotechnology Information at the National Library of Medicine at the National Institutes of Health of the Government of the United States of America, Bethesda, Md., 20894.

In determining whether or not two nucleotide sequences fall within a particular percentage identity limitation recited herein, those skilled in the art will be aware that it is necessary to conduct a side-by-side comparison or multiple alignment of sequences. In such comparisons or alignments, differences may arise in the positioning of non-identical residues, depending upon the algorithm used to perform the alignment. In the present context, reference to a percentage identity between two or more nucleotide sequences shall be taken to refer to the number of identical residues between said sequences as determined using any standard algorithm known to those skilled in the art.

For example, nucleotide sequences may be aligned and their identity calculated using the BESTFIT program or other appropriate program of the Computer Genetics Group, Inc., University Research Park, Madison, Wis., United States of America (D)evereaux et al, Nucl. Acids Res. 12, 387-395, 1984). As discussed supra BLAST is also useful for aligning nucleotide sequences and determining percentage identity.

In another example of the invention, the membrane transport protein is a cystic fibrosis transmembrane regulator (CFIR) protein. As used herein the term “cystic fibrosis transmembrane regulator protein” or “CFTR” shall be taken to mean a protein that comprises an amino acid sequence at least about 80% identical to the sequence set forth in SEQ ID NO: 36. For example, the protein comprises an amino acid sequence at least about 85% or at least about 90% or at least about 95% or at least about 98% or at least about 99% identical to the amino acid sequence set forth in SEQ ID NO: 36.

In one example, the CFTR protein is a human CFTR protein.

Alternatively, or in addition, a CFTR protein is a protein encoded by a nucleic acid that comprises a nucleotide sequence at least about 80% identical to the nucleotide sequence set forth in SEQ ID NO: 35. For example, the nucleic acid comprises a nucleotide sequence at least about 85% or at least about 90% or at least about 95% or at least about 98% or at least about 99% identical to the nucleotide sequence set forth in SEQ ID NO: 35.

In one form of the invention, the CFTR protein is a mutant CFTR protein. For example, a CFMR mutation selected from the group consisting of 1717-1G→A, G542X, W1282X, N1303K, ΔF508, 3849+10 kb C→T, 621+1 G→T, R553X, G551D, R117H, R1162X and R334W. For example, a CFTR protein comprising a ΔF508 mutation comprises an amino acid sequence set forth in SEQ ID NO: 61.

In another example of the invention the membrane transport protein is a mutant membrane transport protein. As used herein, the term “mutant membrane transport protein” shall be taken to mean a membrane transport protein that comprises one or more amino acid substitutions, insertions or deletions compared to a wild-type form of a membrane transport protein, e.g. a form of a membrane transport protein described supra. While it is not a requirement that the mutant membrane transport is functional, it is beneficial that the membrane transport protein is capable of translocating to a plasma membrane to some degree.

For example, a mutant membrane transport protein has a reduced rate of transporter internalization. As used herein, the term “reduced rate of transporter internalization“shall be taken to mean that has been mutated in such a way that following translocation to the membrane it is not internalized or endocytosed, i.e. translocated away from the membrane at the same rate as the wild-type form of the membrane transport protein, rather it is internalized at a slower rate. For example, a mutant form of GLUT4 that has a reduced rate of transporter internalization includes the L489, 490A mutant (SEQ ID NO: 7) or the F5A mutant (SEQ ID NO: 9). Such a mutant is of use in the process of the present invention as it accumulates at the plasma membrane, effectively amplifying or increasing the level of membrane transport protein detected. Accordingly, such a mutant is useful for detection of a minor change (i.e. increase or decrease) of the translocation of a membrane transport protein, for example, when screening for a modulator of translocation of a membrane transport protein.

In the case of GLUT4, wild-type GLUT4 is more effectively translocated and recycled in the presence of insulin, as would be expected. Accordingly, wild-type GLUT4 is more effective in an assay for determining changes in translocation in the presence and/or absence of insulin, for example, when screening for a compound/agent that modulates GLUT4 translocation in the presence of insulin.

In one example of the invention, the membrane transport protein is a membrane transport protein that is rapidly translocated and recycled, whether that membrane transport protein is a wild-type or mutant form.

Detectable Labels

In an example of the invention, the membrane transport protein is labeled. For example, with a detectable label. Accordingly, the present invention provides a process for determining the level of a labeled membrane transport protein translocated to the plasma membrane of a cell expressing the labeled membrane transport protein, said process comprising:
(a) determining the level of the labeled membrane transport protein at the plasma membrane of a cell using a method comprising:
   (i) contacting the cell with a ligand that binds to the label for a time and under conditions sufficient for the ligand to bind the label; and
   (ii) determining the level of ligand bound to the labeled membrane transport protein;
(b) (i) permeabilizing or disrupting the plasma membrane of a cell and contacting the labeled membrane transport protein within the cell with the ligand of the label for a time and under conditions sufficient for the ligand to bind the label; and
   (ii) determining the level of ligand bound to the labeled membrane transport protein within the cell; and
(c) comparing the level of ligand detected at (a) (ii) and (b) (ii) to determine the level of the labeled membrane transport protein at the plasma membrane relative to the level of the labeled membrane transport protein inside the cell.

For example, the label is a peptide, polypeptide or protein that is heterologous to the membrane transport protein. Such a label facilitates detection of the membrane transport protein with which the peptide, polypeptide or protein is associated.

A suitable detectable label includes, for example, a peptide, polypeptide or protein to which an antibody or ligand is capable of specifically binding. Alternatively, or in addition, the label is, for example, an enzyme that catalyzes a detectable reaction when contacted with a suitable substrate.

An example of a suitable detectable peptide polypeptide or protein is selected from the group consisting of influenza virus hemaglutinin (HA) (SEQ ID NO: 15), Simian Virus 5 (V5) (SEQ ID NO: 16), polyhistidine (SEQ ID NO: 17), c-myc (SEQ ID NO: 18), FLAG (SEQ ID NO: 19), an epitope tag described by Sloostra et al., Mol. Drivers 2:156-164 (SEQ ID NO: 20 or SEQ ID NO: 21), GST (SEQ ID NO: 22), MBP (SEQ ID NO: 23), GAL4 (SEQ ID NO: 24), β-galactosidase (SEQ ID NO: 25), enhanced green fluorescence protein (eGFP) (SEQ ID NO: 26), yellow fluorescent protein (SEQ ID NO: 27), soluble modified blue fluorescent protein (SEQ ID NO: 28), soluble-modified red-shifted green fluorescent protein (SEQ ID NO: 29) and cyan fluorescent protein (SEQ ID NO: 30).

Alternatively, the membrane transport protein is labeled with a protein that directly associates with another known protein, such as for example, biotin, strepavidin or the Strep-Tag, an 8 amino acid strepavidin binding sequence (WSHPQFEK, SEQ ID NO: 31) (available from Sigma-Genosys, Sydney, Australia).

In an exemplified embodiment of the invention, the label that is linked to a membrane transport protein is a HA tag (SEQ ID NO: 15).

In one form of the invention, the label is linked or fused to an extracellular domain of a membrane transport protein. Accordingly, it is preferable that the labeled membrane transport protein is a fusion protein. As used herein, the term “extracellular domain” shall be taken to mean the region or component of a protein that is located external to the cell when the membrane transport protein is incorporated in to the plasma membrane. Accordingly, when a membrane transport protein is not incorporated into the plasma membrane of a cell, the extracellular domain may be located within the cell.

Methods for determining the subcellular localization of a domain of a protein are known in the art. For example the following programs are useful for determining an extracellular domain of a protein:
i) PSORT, based on Horton and Nakai Proc Int Conf Intell Syst Mol Biol.;5: 147-52, 1997) is available from the Brinloan Laboratory at Simon Fraser University, Burnaby, British Columbia, Canada;
ii) TopPred 2 based on Gunnar von Heijne, J. Mol. Biol. 225, 487-494, 1992 available from Stockholm University;
iii) HMMTOP based on Tusnady and Simon J. Mol. Biol. 283: 489-506, 1998 available from The Institute of Enzymology, Hungarian Academy of Sciences, Budapest; and
iv) SOSUI available from Department of Biotechnology, Tokyo University of Agriculture and Technology.

Alternatively, or in addition, a region of a membrane transport protein that is extracellular is predicted using the method described, for example, in Nakashima and Nishikawa, FEBS Lett. 303: 141-146, 1992; Nakashima and Nishikawa, J. Mol. Biol., 238: 54-61, 1994; Rost et al, Prot Sci., 4: 521-533, 1995; or Chou and Cai, Biochem Biophys Res Commun. 320:1236-9, 2004. Such methods rely upon the analysis of the amino acid composition of a membrane transport protein to determine, for example, hydropathy of regions of the protein to determine a region that is extracellular or intracellular.

In an exemplified form of the invention, the tag is linked or fused to the first exofacial or extracellular loop of the GLUT4 protein or a mutant thereof. For example. This protein comprises the sequence set forth in SEQ ID NO: 4 and/or is encoded by a nucleic acid set forth in SEQ ID NO: 3. A labeled TAIL mutant of GLUT4 comprises, for example, the sequence set forth in SEQ ID NO: 6. A labeled L489, 490A mutant of GLUT4 comprises, for example, the sequence set forth in SEQ ID NO: 8. A labeled F5A mutant of GLUT4 comprises, for example, the sequence set forth in SEQ ID NO: 10.

In an example of the invention, the label is covalently linked to the membrane transport protein. For example, a disulfide bond is formed between the label and the membrane transport protein. As will be apparent to the person skilled in the art such a membrane transport protein is then be delivered to the cell. In one embodiment the peptide encoded by the nucleic acid fragment of the present invention is expressed as a fusion protein with a peptide sequence capable of enhancing, increasing or assisting penetration or uptake of the protein by cells. Means and methods of enhancing, increasing or assisting penetration or uptake of the membrane transport protein by cells are described, for example, In Morris et al, Nature Biotechnology 19, 1173-1176, 2001.

In an alternative example, the membrane transport protein is expressed as a fusion protein with the label (e.g., as a recombinant fusion protein). As will be apparent to the skilled artisan, a fusion protein is advantageously expressed within a cell using an expression construct. As used herein, the term “expression construct” is to be taken in its broadest context and includes a promoter sequence that is placed in operable connection with a nucleic acid that encodes a membrane transport protein (e.g., a labeled membrane transport protein) of the present invention.

The term “promoter” is to be taken in its broadest context and includes the transcriptional regulatory sequences of a genomic gene, including the TATA box or initiator element, which is required for accurate transcription initiation, with or without additional regulatory elements (i.e. upstream activating sequences, transcription factor binding sites, enhancers and silencers) which alter gene expression in response to developmental and/or external stimuli, or in a tissue specific manner. In the present context, the term promoter” is also used to describe a recombinant, synthetic or fusion molecule, or derivative which confers, activates or enhances the expression of a nucleic acid molecule to which it is operably linked, and which encodes the peptide or protein. Preferred promoters can contain additional copies of one or more specific regulatory elements to further enhance expression and/or alter the spatial expression and/or temporal expression of said nucleic acid molecule.

Placing a nucleic acid molecule under the regulatory control of, i.e., “in operable connection with”, a promoter sequence means positioning said molecule such that expression is controlled by the promoter sequence. Promoters are generally positioned 5′ (upstream) to the coding sequence that they control. To construct heterologous promoter/structural gene combinations, it is generally preferred to position the promoter at a distance from the gene transcription start site that is approximately the same as the distance between that promoter and the gene it controls in its natural setting, i.e., the gene from which the promoter is derived. As is known in the art, some variation in this distance can be accommodated without loss of promoter function. Similarly, the preferred positioning of a regulatory sequence element with respect to a heterologous gene to be placed under its control is defined by the positioning of the element in its natural setting, i.e., the gene from which it is derived. Again, as is known in the art, some variation in this distance can also occur.

Typical promoters suitable for expression in a virus of a mammalian cell, or in a mammalian cell, mammalian tissue or intact mammal include, for example a promoter selected from the group consisting of, a retroviral LTR element, a SV40 early promoter, a SV40 late promoter, a cytomegalovirus (CMV) promoter, a CMV IE (cytomegalovirus immediate early) promoter, an EFI, promoter (from human elongation factor 1α), an EM7 promoter or an UbC promoter (from human ubiquitin C).

Typical promoters suitable for expression in viruses of bacterial cells and bacterial cells such as for example a bacterial cell selected from the group comprising E. coli, Staphylococcus sp, Corynebacterium sp., Salmonella sp., Bacillus sp., and Pseudomonas sp., include, but are not limited to, the lacz promoter, the Ipp promoter, temperature-sensitive λ_{L} or λ_{R} promoters, T7 promoter, T3 promoter, SP6 promoter or semi-artificial promoters such as the IPTG-inducible tac promoter or lacLT5 promoter. A number of other gene construct systems for expressing the nucleic acid fragment of the invention in bacterial cells are well-known in the art and are described for example, in Ausubel et al (In: Current Protocols in Molecular Biology. Wiley Interscience, ISBN 047 150338, 1987) and (Sambrook et al (In: Molecular Cloning: Molecular Cloning: A Laboratory Manual, Cold Spring Harbor Laboratories, New York, Third Edition 2001).

Typical promoters suitable for expression in yeast cells such as for example a yeast cell selected from the group comprising Pichia pastoris, S. cerevisiae and S. pombe, include, but are not limited to, the ADH1 promoter, the GAL1 promoter, the GAL4 promoter, the CUP1 promoter, the PHO5 promoter, the nmt promoter, the RPR1 promoter, or the TEF1 promoter.

Methods for producing expression constructs are known in the art and are described, for example, in Ausubel et al (In: Current Protocols in Molecular Biology. Wiley Interscience, ISBN 047 150338, 1987) or Sambrook et al (In: Molecular Cloning: Molecular Cloning: A Laboratory Manual, Cold Spring Harbor Laboratories, New York, Third Edition 2001).

In one embodiment, the expression construct forms a component of an expression vector. The term “expression vector” refers to a nucleic acid molecule that has the ability to confer expression on a nucleic acid to which it is operably connected, in a cell or in a cell free expression system. Within the context of the present invention, it is to be understood that an expression vector may comprise a promoter as defined herein, a plasmid, bacteriophage, phagemid, cosmid, virus sub-genomic or genomic fragment, or other nucleic acid capable of maintaining and or replicating heterologous DNA in an expressible format. Many expression vectors are commercially available for expression in a variety of cells. Selection of appropriate vectors is within the knowledge of those having skill in the art.

For example, expression vectors that contain suitable promoter sequences for expression in mammalian cells or mammals include, but are not limited to, the pcDNA vector suite supplied by Invitrogen, the pCI vector suite (Promega), the pCMV vector suite (Clontech), the pM vector (Clontech), the pSI vector (Promega) or the VP16 vector (Clontech).

Numerous expression vectors for expression of recombinant polypeptides in bacterial cells and efficient ribosome binding sites have been described, such as for example, PKC30 (Shimatake and Rosenberg, Nature 292, 128, 1981); pKK173-3 (Amann and Brosius, Gene 40, 183, 1985), pET-3 (Studier and Moffat, J. Mol. Biol. 189, 113, 1986); the pCR vector suite Invitrogen), pGEM-T Easy vectors (Promega), the pL expression vector suite (nvitrogen) the pBAD/TOPO Invitrogen, Carlsbad, Calif.); the pFLEX series of expression vectors (Pfizer Inc., Conn., USA); the pQE series of expression vectors (QIAGEN, Calif., USA), or the pL series of expression vectors Invitrogen), amongst others.

Expression vectors for expression in yeast cells are know in the art and include, but are not limited to, the pACT vector (Clontech), the pDBleu-X vector, the pPIC vector suite Invitrogen), the pGAPZ vector suite Invitrogen), the pHYB vector (Invitrogen), the pYD1 vector (Invitrogen), and the pNMT1, pNMT41, pNMT81 TOPO vectors (Invitrogen), the pPC86-Y vector (Invitrogen), the pRH series of vectors Invitrogen), pYESTrp series of vectors (Invitrogen).

Following production of a suitable gene construct, said construct is introduced into the relevant cell. Methods of introducing the gene constructs into a cell or organism for expression are well known to those skilled in the art and are described for example, in Ausubel et al (In: Current Protocols in Molecular Biology. Wiley Interscience, ISBN 047 150338, 1987) and (Sambrook et al (In: Molecular Cloning: Molecular Cloning: A Laboratory Manual, Cold Spring Harbor Laboratories, New York, Third Edition 2001). The method chosen to introduce the gene construct in depends upon the cell type in which the gene construct is to be expressed. Means for introducing recombinant DNA into bacterial cells include, but are not limited to electroporation or chemical transformation into cells previously treated to allow for said transformation, PEG mediated transformation, microinjection, transfection mediated by DEAE-dextran, transfection mediated by calcium phosphate, transfection mediated by liposomes such as by using Lipofectamine Invitrogen) and/or cellfectin (nvitrogen), transduction by Adenoviuses, Herpesviruses, Togaviruses or Retroviruses and microparticle bombardment such as by using DNA-coated tungsten or gold particles (Agacetus Inc., Wis., USA).

As exemplified herein, the present inventors have used a retroviral system to transfect or transduce a cell with an expression construct encoding a membrane transport protein. Accordingly, a viral delivery system is contemplated by the present invention. Conventional viral based systems for the delivery of a nucleic acid include, for example, retroviral, lentivirus, adenoviral, adeno-associated virus and herpes simplex virus. Viral vectors are an efficient and versatile method of gene transfer in target cells and tissues. Integration in the host cell genome occurs with the retrovirus, lentivirus, and adeno-associated virus gene transfer methods, often resulting in long term expression of the inserted expression construct. Additionally, high transduction efficiencies have been observed in many different cell types and target tissues.

The tropism of a retrovirus can be altered by incorporating foreign envelope proteins, expanding the potential target population of target cells. A lentiviral vector is a retroviral vector that is capable of transducing or infecting a non-dividing cell and typically produces high viral titers. Selection of a retroviral gene transfer system depends on the target tissue.

A Retroviral vector comprises cis-acting long terminal repeats (LTRs) with packaging capacity for up to 6-10 kb of foreign sequence. The minimum cis-acting LTRs are sufficient for replication and packaging of the vectors, which are then used to integrate the membrane transport gene into the target cell to provide long term transgene expression. Widely used retroviral vectors include those based upon murine leukemia virus (MuLV), gibbon ape leukemia virus (GaLV), simian immunodeficiency virus (SIV), human immunodeficiency virus (FIV), and combinations thereof (see, e.g., Buchscher et al., J. Virol. 66:2731-2739 (1992); Johann et al., J. Virol. 66:1635-1640 (1992); Sommerfelt et al., Virol. 176:58-59 (1990); Wilson et al., J. Virol. 63:274-2378 (1989); Miller et al., J. Virol. 65:2220-2224 (1991); PCT/US94/05700; Miller and Rosman BioTechniques 7:980-990, 1989; Miller, A. D. Human Gene Therapy 1:5-14, 1990; Scarpa et al) Virology 180:849-852, 1991; Burns et al. Proc. Natl. Acad. Sci. USA 90:8033-8037, 1993.).

In applications where transient expression of the nucleic acid is preferred, adenoviral based systems are typically used. Adenoviral based vectors are capable of high transduction efficiency in many cell types and do not require cell division. With such vectors, high titer and levels of expression have been obtained. This vector can be produced in large quantities in a relatively simple system. (see, e.g., West et al., Virology 160:3847 1987; U.S. Pat. No. 4,797,368; WO 93/24641; Kotin, Human Gene Therapy 5:793-8011994; Muzyczka Clin. Invest. 94:1351 1994).

Various adeno-associated virus (AAV) vector systems have also been developed for nucleic acid delivery. AAV vectors can be readily constructed using techniques known in the art. See, e.g., U.S. Pat. Nos. 5,173,414 and 5,139,941; International Publication Nos. WO 92/01070 and WO 93/03769; Lebkowski et al. Molec. Cell. Biol. 8:3988-3996, 1988; Vincent et al. (1990) Vaccines 90 (Cold Spring Harbor Laboratory Press); Carter Current Opinion in Biotechnology 3:533-539, 1992; Muzyczka Current Topics in MicrobioL and ImmunoL 158:97-129, 1992; Kotin, Human Gene Therapy 5:793-801, 1994; Shelling and Smith Gene Therapy 1:165-169, 1994; and Zhou et al. J. Exp. Med. 179:1867-1875, 1994.

Additional viral vectors useful for delivering a nucleic acid encoding membrane transport protein by gene transfer include those derived from the pox family of viruses, such as vaccinia virus and avian poxvirus or an alphavirus or a conjugate virus vector (e.g. that described in Fisher-Hoch et al., Proc. Natl. Acad. Sci. USA 86:317-321, 1989).

As will be apparent from the preceding description, the present invention also encompasses providing the cell that expresses a membrane protein. The term “providing the cell that expresses a membrane protein” shall be taken to include transforming, transfecting or transducing a cell with an expression construct that encodes the membrane transport protein. Optionally, the term “providing the cell that expresses a membrane protein” shall be taken to additionally mean preparing the expression construct that encodes the membrane transport protein.

Suitable Cells

As membrane transfer proteins are found in the majority of species any cell that expresses a membrane transport protein in nature is suitable for the performance of the instant invention. For example, transporters, channels and primary active transporters are found in bacterium, yeast, plants and mammals, see, for example, Chung et al., Journal of Bacteriology, 183: 1012-1021, 2001. Furthermore, ABC transport proteins are found in bacterium, yeast and mammals.

In an example of the invention, the cell is a eukaryotic cell, for example, a mammalian cell.

As will be apparent to the slilled artisan, the process of the present invention is preferably performed in vitro. Accordingly, the invention is performed, for example, using a cell isolated from a subject or using a cell line.

In one example of the invention, the method is performed in a cell that is amenable to transformation, transfection or transduction. For example, the cell is a cell selected from the group consisting of COS, CHO, murine 10T, MEF, NIH3T3, MDA-MB-231, MDCK, HeLa, K562, HEK 293, 3T3-L1 and 293T.

COS cells have been previously shown to be amenable to both transfection/transduction and the study of translocation of a membrane transport protein, particularly a GLUT4 protein.

In another example, a cell useful for performance of the process of the invention is a cell that is known to express and/or translocate the membrane transport protein of interest in nature. For example, muscle cells and adipocyte cells are known to express and translocate GLUT4 in nature. Accordingly, a muscle cell selected from the group consisting of a C2C12 cell, a L8 cell, a L6 cell, a F3 cell, a 10T1/2 cell, a H9C2 cell and a BC3H cell is useful for the performance of the invention. Alternatively, or in addition, an adipocyte cell or a pre-adipocyte cell selected from the group consisting of a 3T3-L1 cell, a HIB1B cell and a PA26 cell is useful for the performance of the invention.

As GLUT1 is also expressed and translocated in a muscle cell the muscle cells described supra are useful for the performance of the process of the invention to assess the translocation of GLUT4.

The translocation of CFTR is, for example, studied in a cell line derived from a tissue affected in cystic fibrosis, e.g., a Calu-3 airway epithelium cell line or a T84 colonic cell line.

Alternatively, the translocation of a membrane transport protein is studied using a primary cell, i.e. a cell isolated from a subject. For example, methods of isolating an adipocyte, a pre-adipocyte, a fibroblast, a muscle cell or an airway epithelium cell are known in the art. For example, Katoh et al., Folia Histochem Cytobiol. 32:235-8, 1994 describe a method for isolating a pre-adipocyte cell from adipose tissue.

Detection of a Membrane Transport Protein

To determine the level of a membrane transport protein at the plasma membrane of a cell, a ligand is selected that is capable of specifically binding the membrane transport, for example, a ligand capable of binding to the label of a labeled membrane transport protein.

As used herein the term “ligand” shall be taken in its broadest context to include any chemical compound, polynucleotide, peptide, protein, lipid, carbohydrate, small molecule, natural product, polymer, etc. that is capable of selectively binding, whether covalently or not, to one or more specific sites on a target molecule, e.g., a labeled membrane transport protein (e.g., a label associated with or bound to the membrane transport protein). The ligand may bind to its target via any means including hydrophobic interactions, hydrogen bonding, electrostatic interactions, van der Waals interactions, pi stacking, covalent bonding, or magnetic interactions amongst others.

In one example of the invention, the ligand is an antibody. As used herein the term “antibody” refers to intact monoclonal or polyclonal antibodies, immunoglobulin (IgA, IgD, IgG, IgM, IgE) fractions, humanized antibodies, or recombinant single chain antibodies, as well as fragments thereof, such as, for example Fab, F(ab)2, and Fv fragments.

Antibodies referred to herein are obtained from a commercial source, or alternatively, produced by conventional means. Commercial sources will be known to those skilled in the art. For example, Sigma-Aldrich (Sydney, Australia) sell monoclonal antibodies that specifically bind HA, FLAG, V5, polyhistidine, c-myc, GST, MBP, β-galactosidase, GFP or biotin. The present inventors have used an anti-HA monoclonal antibody to determine the level of translocation of a HA tagged membrane transport protein (eg., a HA-tagged GLUT4 protein).

High titer antibodies are preferred, as these are more useful commercially in kits for analytical, diagnostic and/or therapeutic applications. By thigh titer” is meant a titer of at least about 1:10³ or 1:10⁴ or 1:10⁵. Methods of determining the titer of an antibody will be apparent to the skilled artisan. For example, the titer of an antibody in purified antiserum may be determined using an ELISA assay to determine the amount of IgG in a sample. Typically an anti-IgG antibody or Protein G is used in such an assay. The amount detected in a sample is compared to a control sample of a known amount of purified and/or recombinant IgG. Alternatively, a kit for determining antibody may be used, e.g. the Easy TITER kit from Pierce (Rockford, Ill., USA).

Antibodies may be prepared by any of a variety of techniques known to those of ordinary skill in the art, and are described, for example in, Harlow and Lane (In: Antibodies: A Laboratory Manual, Cold Spring Harbor Laboratory, 1988). In one such technique, an immunogen comprising the antigenic polypeptide is initially injected into any one of a wide variety of animals (e.g., mice, rats, rabbits, sheep, humans, dogs, pigs, chickens and goats). The immunogen is derived from a natural source, produced by recombinant expression means, or artificially generated, such as by chemical synthesis (e.g., BOC chemistry or FMOC chemistry).

A peptide, polypeptide or protein is optionally joined to a carrier protein, such as bovine serum albumin or keyhole limpet hemocyanin. The immunogen and optionally a carrier for the protein is injected into the animal host, preferably according to a predetermined schedule incorporating one or more booster immunizations, and blood collected from said the animals periodically. Optionally the immunogen is injected in the presence of an adjuvant, such as, for example Freund's complete or incomplete adjuvant, lysolecithin and/or dinitrophenol to enhance the ;immune response to the immunogen. Monoclonal or polyclonal antibodies specific for the polypeptide are then be purified from the blood isolated from an animal by, for example, affinity chromatography using the polypeptide coupled to a suitable solid support.

Monoclonal antibodies specific for the antigenic polypeptide of interest may be prepared, for example, using the technique of Kohler and Milstein, Eur. J. Immunol. 6:511-519, 1976, and improvements thereto. Briefly, these methods involve the preparation of immortal cell lines capable of producing antibodies having the desired specificity (i.e., reactivity with the polypeptide of interest). Such cell lines may be produced, for example, from spleen cells obtained from an animal immunized as described supra. The spleen cells are then immortalized by, for example, fusion with a myeloma cell fusion partner, preferably one that is syngenic with the immunized animal. A variety of fusion techniques may be employed, for example, the spleen cells and myeloma cells may be combined with a nonionic detergent or electrofused and then grown in a selective medium that supports the growth of hybrid cells, but not myeloma cells. A preferred selection technique uses HAT (hypoxanthine, aminopterin, thymidine) selection. After a sufficient time, usually about 1 to 2 weeks, colonies of hybrids are observed. Single colonies are selected and growth media in which the cells have been grown is tested for the presence of binding activity against the polypeptide (immunogen). Hybridomas having high reactivity and specificity are preferred.

Monoclonal antibodies are isolated from the supernatants of growing hybridoma colonies using methods such as, for example, affinity purification as described supra. In addition, various techniques may be employed to enhance the yield, such as injection of the hybridoma cell line into the peritoneal cavity of a suitable vertebrate host, such as a mouse. Monoclonal antibodies are then harvested from the ascites fluid or the blood of such an animal subject. Contaminants are removed from the antibodies by conventional techniques, such as chromatography, gel filtration, precipitation, and/or extraction.

It is preferable that an immunogen used in the production of an antibody is one which is sufficiently antigenic to stimulate the production of antibodies that will bind to the immunogen and is preferably, a high titer antibody. For example, an immunogen may be an entire protein.

Alternatively, an immunogen consists of a peptide representing a fragment of a polypeptide. Preferably, an antibody raised to such an immunogen also recognizes the filll-length protein from which the immunogen was derived, such as, for example, in its native state or having native conformation.

As discussed supra antibody fragments are contemplated by the present invention. The term “antibody fragment” refers to a portion of a full-length antibody, generally the antigen binding or variable region. Examples of antibody fragments include Fab, Fab′, F(ab′)₂ and Fv fragments.

Papain digestion of an antibody produces two identical antigen binding fragments, called the Fab fragment, each with a single antigen binding site, and a residual “Fc” fragment.

Pepsin treatment yields an F(ab′)₂ fragment that has two antigen binding fragments that are capable of cross-lng antigen, and a residual other fragment (which is termed pFc′). Additional fragments can include diabodies, linear antibodies, single-chain antibody molecules, and multispecific antibodies formed from antibody fragments. As used herein, “functional fragment” with respect to antibodies, refers to Fv, F(ab) and F(ab′)₂ fragments.

An “Fv” fragment is the minimum antibody fragment that contains a complete antigen recognition and binding site. This region consists of a dimer of one heavy and one light chain variable domain in a non-covalent association (V_{H}-V_{L} dimer). It is in this configuration that the three CDRs of each variable domain interact to define an antigen binding site on the surface of the V_{H}-V_{L} dimer. Collectively, the six CDRs confer antigen binding specificity to the antibody. However, even a single variable domain (or half of an Fv comprising only three CDRs specific for an antigen) has the ability.to recognize and bind antigen.

A Fab fragment [also designated as F(ab)] also contains the constant domain of the light chain-and the first constant domain (CHi) of the heavy chain. Fab′ fragments differ from Fab fragments by the addition of a few residues at the carboxyl terminus of the heavy chain CH1 domain including one or more cysteines from the antibody hinge region. F(ab′) fragments are produced by cleavage of the disulfide bond at the hinge cysteines of the F(ab′)₂ pepsin digestion product. Additional chemical couplings of antibody fragments are known to those of ordinary skill in the art.

“Single-chain Fv” or “scFv” antibody fragments comprise the VH and VL domains of an antibody, wherein. these, domains are present in a single polypeptide chain. Generally, the Fv polypeptide further comprises a polypeptide linker between the VH and VL domains which enables the scFv to form the desired structure for antigen binding. For a review of scFv,. see Pluckthun in The Pharmacology of Monoclonal Antibodies, vol. 113, Rosenburg and Moore eds. Springer-Verlag, New York, pp. 269-315 (1994).

In another example, a ligand is a small molecule. Chemical small molecule libraries are available commercially or alternatively may be generated using methods known in the art, such as, for example, those described in U.S. Pat. No. 5,463,564.

Alternatively, a ligand is a peptidyl ligand. A peptidyl ligand are conveniently made by standard peptide synthesis, such as the Merrifield method of synthesis (Merrifield, J Am Chem Soc, 85,:2149-2154, 1963) and the myriad of available improvements on that technology (see e.g., Synthetic Peptides: A User's Guide, Grant, ed. (1992) W. H. Freeman & Co., New York, pp. 382; Jones (1994) The Chemical Synthesis of Peptides, Clarendon Press, Oxford, pp. 230.).

For example, a membrane transport protein is labeled with strepavidin and the peptidyl ligand is a peptide that comprises a strepavidin binding sequence, e.g. the amino acid sequence set forth in SEQ ID NO: 31.

Alternatively, the membrane transport protein is labeled with biotin and the ligand is strepavidin.

As will be apparent to the skilled artisan, a preferred ligand is not capable of independently entering a cell that has not been permeablilized or disrupted. Accordingly, when a cell with an intact plasma membrane is contacted with the ligand, said ligand will bind to the membrane transport protein in the plasma membrane, and not to the membrane protein within the cell to a significant degree.

However, the present inventors have shown that the ligand may be capable of entering the cell when bound to a membrane transport protein that recycles away from the membrane without significantly altering the efficacy of the test. In fact, such a ligand is useful for determining internalization and/or a rate of internalization of a membrane transport protein.

A ligand useful in the process of the present invention is, for example, labeled with a detectable marker. For example, a fluorescent label (e.g. FITC or Texas Red), a fluorescent semiconductor nanocrystal (as described in U.S. Pat. No. 6,306,610), a radiolabel or an enzyme (e.g. horseradish peroxidase (HRP), alkaline phosphatase (AP) or β-galactosidase)

An example of a suitable fluorescent label include fluorescein (FITC), 5,6-carboxymethyl fluorescein, Texas red, nitrobenz-2-oxa-1,3-diazol-4-yl (NBD), coumarin, dansyl chloride, rhodamine, 4′-6-diamidino-2-phenylinodole (DAPI), and the cyanine dyes Cy3, Cy3.5, Cy5, Cy5.5 and Cy7, fluorescein (5-carboxyfluorescein-N-hydroxysuccinimide ester), rhodamine (5,6-tetramethyl rhodamine). The absorption and emission maxima, respectively, for these fluors are: FITC (490 nm; 520 nm), Cy3 (554 nm; 568 nm), Cy3.5 (581 nm; 588 nm), Cy5 (652 nm: 672 nm), Cy5.5 (682 nm; 703 nm) and Cy7 (755 nm; 778 nm).

In an exemplified form of the invention a suitable fluorescent label is, for example, a fluorescent label obtained from Molecular Probes, Eugene. Oreg., such as, for example Alexafluor®350, Alexafluor® 488, Alexafluor® 555, Alexafluor® 594 or Alexafluor® 647. Such an antibody may be purchased from a commercial source. Alternatively, Molecular Probes supplies kits for labeling an antibody or proteinaceous ligand with such a fluorescent label.

In another example, the label is a fluorescent nanocrystal. A fluorescent nanocrystal generally comprises a core composed of cadmium sulfide (CdS), cadmium selenide (CdSe), or cadmium telluride (CdTe). The size and shape of the core aids in determining the wavelength at which the nanocrystal fluoresce. Coating the core is a shell composed of a non-emissive transparent but structurally related material, for example, ZnS. Finally, such a fluorescent nanocrystal is coated to provide a carboxylate surface to which many biological and nonbiological moieties may be attached. Such a nanocrystal is then conjugated to a ligand of interest, eg., an antibody, for example using an antibody conjugation kit from Qdot® (Hayward, Calif.). By exciting the nanocrystal at the relevant wavelength, the crystal emits a fluorescent light that is detectable using a method known in the art and/or described herein.

In a further example, the label is an enzymatic label. For example, a ligand is conjugated to β-galactosidase. Following contacting the cell and/or membrane transport protein with such a ligand, the sample is contacted with, for example, 5-bromo-4-chloro-3-indol-beta-D-galaotopyranoside (x-gal). The resulting reaction causes a blue colored precipitate to form. Other enzymatic labels are know in the art and include, for example, alkaline phosphatase or horseradish peroxidase (HRP). Suitable substrates for such enzymes are known in the art and include, for example, hydrogen peroxide or 3-3,5,5′-tetramethylbenzidine (TMB).

In another example, the ligand that binds to the label is detected using another ligand, such as, for example, an antibody. For example the secondary antibody/ligand is capable of specifically binding to the ligand that binds to the label. The present inventors have used a mouse monoclonal antibody to bind a labeled membrane transport protein and an anti-mouse secondary antibody to detect binding of the mouse monoclonal antibody. Preferably, the secondary antibody is labeled with a detectable marker, such as, for example, a marker described supra.

Alternatively, a ligand that binds to a label or a secondary antibody/ligand is conjugated to, for example, biotin. Strepavidin is capable of binding to biotin with high affinity and specificity. Accordingly, strepavidin labeled with a detectable marker is useful for detecting the binding of the ligand that binds to a label or a secondary antibody/ligand. A suitable detectable marker will be apparent to the skilled artisan, for example, a marker described supra.

Detection Methods

Methods for detecting the binding of the ligand to the label and/or the secondary antibody/ligand to the primary ligand are known in the art and/or described herein. For example, such detection methods are described in Scopes (In: Protein purification: principles and practice, Third Edition, Springer Verlag, 1994).

In one form of the invention, the level of the ligand bound to the membrane transport protein is determined by a process comprising contacting the ligand with an antibody that specifically binds the label for a time and under conditions sufficient for the antibody to bind and determining the level of bound antibody.

As will be apparent to the skilled artisan, the detection method used depends upon the type of label used.

For example, a standard solid-phase ELISA format is useful in determining the level of an enzyme labeled ligand or antibody.

In one form such an assay involves immobilizing or growing or incubating the cell supra onto a solid matrix, such as, for example a polystyrene or polycarbonate microwell or dipstick, a membrane, or a glass support (e.g. a glass slide). Preferably, the ELISA assay is performed upon the plate upon which the cells are grown.

An antibody or ligand that specifically binds the membrane transport protein or label is brought into direct contact with the cell, and forms a direct bond with any of the membrane transport protein or label present in said sample. This antibody is generally labeled with a detectable reporter molecule, such as for example, an enzyme (e.g. horseradish peroxidase (RRP)), alkaline phosphatase (AP) or β-galactosidase. Alternatively, a second labeled antibody can be used that binds to the first antibody.

Following washing to remove any unbound antibody the detectable marker is detected by the addition of a substrate, such as for example hydrogen peroxide, TMB, or toluidine, or 5-bromochloro-3-indol-beta-D-galaotopyranoside (x-gal).

The level of the membrane transport protein may be determined using a standard curve that has been produced using known quantities of the membrane transport protein (e.g. recombinant membrane transport protein).

In the case of a fluorescent label, a fluorescence linked immunosorbent assay (FLISA) is useful for determining the level of a labeled ligand or antibody in a sample. A FLISA is performed essentially as described supra_for the ELISA assay, however, a substrate is not required to detect the bound labeled ligand or antibody. Rather, following washing to remove any unbound ligand/antibody the sample is exposed to a light source of the appropriate wavelength and the level of fluorescence emitted by each sample determined. A FLISA is also known as an immunofluorescence assay (IFA). The present inventors have clearly exemplified this form of assay.

As will be apparent to the skilled artisan, other detection methods based on an immunosorbent assay are useful in the performance of the present invention. For example, an immunosorbent method based on the description supra using a radiolabel for detection, or a gold label (e.g. colloidal gold) for detection, or a liposome, for example, encapsulating NAD+ for detection (e.g., as described in Kumada et al.,

Journal of Chemical Engineering of Japan, 34: 943-947, 2001) or an acridinium linked immunosorbent assay.

In another example, the level of the labeled ligand or antibody is determined using immunohistochemistry and/or immunofluorescence. For example, a cell or tissue section that is to be analyzed is optionally fixed to stabilize and protect both the cell and the proteins contained within the cell. Preferably, the method of fixation does not disrupt or destroy the antigenicity of the membrane transport protein, thus rendering it undetectable. Methods for fixing a cell are known in the art and include for example, treatment with paraformaldehyde, treatment with alcohol, treatment with acetone, treatment with methanol, treatment with Bouin's fixative and treatment with glutarpidehyde. Following fixation a cell is incubated with a ligand or antibody capable of binding the membrane transport protein. As discussed supra the ligand or antibody may be labeled with a detectable marker. Alternatively, a second labeled antibody that binds to the first antibody can be used to detect the first antibody. Following washing to remove any unbound antibody, the level of ligand or antibody bound to the membrane transport protein is determined using an appropriate means. Means for detecting a label vary depending upon the type of label used and will be apparent to the skilled artisan.

Methods using immunofluoreseence are preferable, as they are quantitative or at least semi-quantitative. Methods of quantitating the degree of fluorescence of a stained cell are known in the art and described, for example, in hmmunohistochemistry (Cuello, 1984 John Wiley and Sons, ASIN 0471900524).

A high-throughput method of immunohistochemical/immunofluorescent analysis of a biological sample are preferred. For example, the EIDAQ 100-HTM system of Q3DM (San Diego, Calif., USA) allows the rapid automatic analysis of a biological sample to determine the presence and/or level of a polypeptide of interest.

Determining the Level of a Membrane Transport Protein Within a Sample

Following determining the level of membrane transport protein that has translocated to the plasma membrane of a cell, the total amount of that membrane transport protein in the cell is determined using a method known in the art and/or described herein

Accordingly, comparison of the level of the membrane transport protein that has translocated to the plasma membrane to the level of the membrane transport protein detected in the cell provides a relative estimate of the level of the membrane transport protein that has translocated to the plasma membrane as a function of total membrane transport protein (for example as a percentage of total membrane transport protein). Such an estimate effectively “normalizes” the results of such an assay, reducing inter-assay variability and allowing comparisons between multiple assays.

To determine the total amount of membrane transport protein in a cell, the plasma membrane is permeablilized or disrupted to allow the detection means, e.g. a ligand or antibody, to enter the cell and bind the membrane transport protein. In permeabilizing or disrupting a cell membrane it is important that the membrane transport protein within the cell is not significantly degraded.

Methods for permeabilizing a cell are IIown in the art and/or described herein.

For example, a cell or plasma membrane is contacted with an agent or compound that permeabilizes or disrupts a membrane for a time and under conditions sufficient for permeabilization or disruption to occur.

A suitable agent or compound that permeabilizes or disrupts a plasma membrane will be apparent to the skilled artisan. For example, a suitable agent or compound that permeabilizes or disrupts a plasma membrane is selected from the group consisting of saponin, n-octyl-glucopyranoside, n-Dodecyl β-D-maltoside, N-Dodecanoyl-N-methylglycine sodium salt, hexadecyltrimethylammonium bromide, deoxycholate, a non-ionic detergent, streptolysin-O (SEQ ID NO: 32), α-hemolysin (SEQ ID NO: 33), tetanolysin (SEQ ID NO: 34) and mixtures thereof.

Agents useful for disrupting or permeabilizing a membrane are commercially available from, for example, Sigma-Aldrich, Sydney, Australia. For example, saponin, n-octyl-glucopyranoside, n-Dodecyl β-D-maltoside, hexadecyltrimethylammonium bromide, streptolysin-O , α-hemolysin or tetanolysin are commercially available from Sigma Aldrich.

The present inventors contacted a cell with a suitable amount of saponin for a time and under conditions suitable to disrupt or permeabilize a plasma membrane. This method permeablilized the plasma membrane sufficiently to facilitate detection of the level of membrane transport protein within the cell.

Methods for using other agents for permeabilizing a plasma membrane will be apparent to the skilled artisan. For example, Palmer et al., EMBO J 17: 1598-1605, 1998 describe the use of Streptolysin-O to disrupt or permeabilize the membrane of a cell. Gariglio FEBS Lett. 44, 330, 1974, described the use of N-Dodecanoyl-N-methylglycine sodium salt for the lysis of eukaryotic cells.

In an example of the invention a cell is fixed. Methods for fixing a cell are known in the art and/or described herein. In one example, the cell is fixed using a process comprising contacting a cell with a fixative for a time and under conditions suitable for cell fixation to occur.

Fixing a cell ensures that the contents of the cell are less likely to be degraded and/or maintain their native conformation thereby facilitating detection.

A suitable compound for fixing a cell will be apparent to the skilled artisan and includes, for example, a compound selected from the group consisting of formaldehyde, paraformaldehyde, alcohol, methanol, glutaraldehyde, Bouin's fixative and mixtures thereof

In one example of the invention, a cell is fixed at substantially the same time as the cell is permeablilized or disrupted. In another example, the cell is fixed prior to or after the cell is permeablilized or disrupted. In a further example, the cell is fixed in the absence of permeabilization or disruption.

Following permeabilization and/or fixation the level of a membrane transport protein is determined using a method known in the art and/or described supra.

Following determining the level of a membrane transport protein in a cell that comprises a membrane that has been permeablilized or disrupted, the level of the membrane protein at the surface of the protein relative to the level of membrane protein in a cell is determined. Accordingly, such a process enables a quantitative measurement of the level of a membrane transport protein that has translocated to the plasma membrane of a cell.

By determining the level of a membrane transport protein at the plasma membrane of a cell relative to or as a function of the level of the membrane transport protein in the cell, the process of the invention effectively standardizes or normalizes the detected levels of protein. The assay normalizes the level of translocated membrane transport protein based on the level of membrane transport protein in the assay. Such normalization facilitates comparison of results attained in separate/distinct assays.

Should the assay be performed using a plurality of cells, the assay may additionally be normalized, for example, for cell number. Such normalization accounts for variation in the number of cells in an assay (a variable that may affect the level of membrane protein detected in the assay).

Methods for determining cell number are known in the art, and include, for example, manually counting the number of cells used in an assay, or, alternatively, counting a fraction of the number of cells used in an assay. For example, when using a microtitre plate, the number of cells in a fraction of the total area of the plate (eg. 10% or 25% or 50%) of each well of the plate is counted, and this result used to estimate the number of cells in each well of the plate.

Alternatively, or in addition, a sample is normalized for cell number by detecting a protein that is expressed by the cells used in the assay. A protein useful in such an assay is one that is not affected by any conditions, eg., compounds, to which the cells are exposed. For example, should the cells be exposed to various concentrations of a compound, a protein that is affected by the compound (i.e., the expression levels of the protein) is not useful for normalization. Various proteins useful for normalization are known in the art and include, for example, β-tubulin, actin, glyceraldehyde 3-phosphate dehydrogenase (GAPDH), β2 microglobulin, hydroxy-methylbilane synthase, hypoxanthine phosphoribosyl-transferase 1 (BPRT), ribosomal protein L13c, succinate dehydrogenase complex subunit A and TATA box binding protein (11BP).

Methods for determining the level of a protein are described supra and are to be taken to apply mutatis mutandis to the detection of a control protein for normalization. For example, the level of a control protein for normalization is determined using an antibody based assay.

In one example of the invention, the number of cells in a sample is determined by a method comprising contacting the cells with an antibody or ligand capable of binding to a component of the cell for a time and under conditions to occur and determining the level of antibody or ligand bound to the cells, wherein the level of antibody or ligand bound to the cells is indicative of cell number.

Antibodies capable of binding to such control proteins are known in the art. For example, an anti-p-tubulin monoclonal antibody is available from Sigma-Aldrich (Sydney, Australia), as is an anti-actin polyclonal antibody or an anti-β2 microglobulin monoclonal antibody.

As the, control proteins for normalization described supra are intracellular, such normalization is, for example, performed following disruption or permeabilization of the plasma membrane.

Alternatively, or in addition, the sample is normalized for cell number using a compound capable of passing across a cell membrane. For example, a DNA binding molecule, such as, for example Hoechst 33342, is capable of staining DNA in a cell with an intact plasma membrane. Clearly such a nucleic acid stain is also useful for normalization of a cell with a disrupted or permeablilized membrane. Alternative nucleic acid stains include, for example, propidiumn-iodide, 4′ 6-diamidino-2-phenylindole (DAPI), Mithramycin, 7-Aminoactinomycin D or To-Pro-3.

The present inventors have shown that wheat germ agglutinin (WGA) is also useful for normalization for cell number. WGA is capable of binding N-acetylglucosamine or chitobiose. Both of these sugar structures are common to plasma membranes of many cells. Accordingly, WGA is useful for determining cell number or normalizing for cell number using either an undisrupted/unpermeabilized cell or a disrupted/permeablilized cell.

As will be apparent to the skilled artisan, the method need not determine or estimate the number of cells in a sample. Rather the method, for example, comprises determinng the level of a ligand, antibody or compound used for detecting/estimating/normalizing for cell number in a sample and comparing this level to the level detected in another sample.

Accordingly, a method for normalizing for cell number comprises:
(i) contacting a sample comprising a plurality of cells of the invention with a ligand or antibody capable of binding to a cell or a component thereof for a time and under conditions sufficient for a complex to form between the cell or component thereof and the antibody or ligand and determining the level of the complex; and
(ii) contacting another sample comprising a plurality of cells of the invention with a ligand or antibody capable of binding to a cell or a component thereof for a time and under conditions sufficient for a complex to form between the cell or component thereof and the antibody or ligand and determining the level of the complex, wherein a level of the complex that is similar or comparable in (i) and (ii) indicates that there is a similar or comparable number of cells in the samples.

For example, the level of the complex that is similar or comparable in (i) and (ii) does not vary significantly.

As will be apparent to the skilled artisan the level of the complex detected may also be used to normalize the level of translocated membrane transport protein detected. For example, the level of the translocated membrane transport protein detected is expressed as a function of the level of the complex detected thereby normalizing for approximate cell number.

Induction of Translocation

In an example of the invention, the process additionally comprises inducing translocation of the membrane transport protein. For example, the membrane transport protein is induced to translocate using a method comprising contacting a cell with an amount of peptide, polypeptide or protein sufficient to induce translocation of the membrane transport protein for a time and under conditions sufficient for translocation to occur thereby inducing translocation of the membrane transport protein.

For example, contacting a cell with lactose or sucrose induces translocation of a lactose permease to a plasma membrane. Contacting a cell with a sufficient amount of isoproterenol induces translocation of the SCN5A sodium channel to the plasma membrane. Furthermore, contacting a cell with a secretagogue (e.g., KCl, ionomycin or a phorbol ester) induces translocation of a N-type Ca2+ channel to the plasma membrane of a cell.

Furthermore, the present inventors have shown that contacting a cell expressing a GLUT protein (e.g. a GLUT4 protein) with insulin induces increased translocation of the GLUT protein to the plasma membrane.

The present inventors have additionally demonstrated that by contacting a cell expressing a GLUT protein with an amount of insulin and sucrose to induce translocation enhanced levels of the GLUT protein are translocated to the plasma membrane. For example, levels of the GLUT protein translocated to the plasma membrane of a cell contacted with both sucrose and insulin are enhanced compared to the levels induced in a cell contacted with insulin alone.

Accordingly, the invention provides for induction of translocation of a GLUT protein or a mutant thereof by contacting a cell expressing said GLUT protein or mutant with an amount of insulin sufficient to induce translocation for a time and under conditions sufficient for translocation to occur.

In an example, the cell are additionally contacted with an amount of sucrose sufficient to induce translocation for a time and under conditions sufficient for translocation to occur.

In an example of the invention, a cell is contacted with sucrose and/or insulin in the presence of serum.

In one form of the invention, the cells are contacted with insulin and then contacted with sucrose. For example, the cells are contacted with between about 100 nM insulin and about 700 nM insulin, or between about 200 nM insulin and about 600 nM insulin, or about 200 nM insulin, or about 400 nM insulin or about 600 nM insulin.

Cells with an enhanced level of the membrane transport protein translocated to the plasma membrane are useful for, for example, screening for modulators of translocation of the membrane transport protein. Clearly, such an assay is more sensitive than an assay that does not enhance the level of membrane transport protein at the cell surface. This is because the level of the plasma membrane transport protein at the cell surface is enhanced, thereby facilitating detection.

Furthermore, such an assay is useful for selecting for a potent inhibitor of translocation of a membrane transport protein.

Furthermore, the present inventors have clearly demonstrated that the process of the invention is useful for screening for modulators of the level of translocation of a plasma membrane protein. In particular, the present inventors have demonstrated that contacting a cell with insulin or contacting a cell with insulin and then sucrose are useful for enhancing the level of a GLUT4 protein translocated to the plasma membrane of a cell.

Alternative methods for the induction of translocation of GLUT4 to the plasma membrane include, for example, contacting a cell with a sufficient amount of margatoxin or another voltage-gated K+ channel, Kv1.3 antagonist for a time and under conditions sufficient to suppress expression or activity of voltage-gated K+ channel, Kv1.3. Such suppression of activity (using margatoxin) or expression (using a mouse knock-out) has been shown to increase the level of GLUT4 translocated to the plasma membrane of a cell (Xu et al, Proc Natl Acad Sci USA. 101:3112-3117, 2004.)

Suppression of Translocation

The present inventors have additionally suppressed the level of a membrane transport protein translocated to the plasma membrane of a cell. Such a method is useful for, for example, modeling a disease/disorder or condition that is associated with a reduced or suppressed level of translocation of a plasma membrane protein. This model is then useful for determining a modulator or putative therapeutic of such a disease/disorder or condition.

For example, the present inventors have shown that by incubating cells expressing GLUT4 in the absence of insulin for a time and under conditions sufficient to induce resistance to insulin induced GLUT4 translocation the level of GLUT4 translocated to the plasma membrane of the cell in the presence of insulin is suppressed. For example, a cell is incubated in the presence of insulin for at least about 16 hours to at least about 72 hours prior to induction of translocation or testing of a compound/agent. For example, a cell is incubated in the presence of insulin for at least about 24 hours to at least about 48 hours prior to induction of translocation or testing of a compound/agent. For example, a cell is incubated in the presence of insulin for about 24 hours prior to induction of translocation or testing of a compound/agent. For example, a cell is incubated in the presence of insulin for about 48 hours prior to induction of translocation or testing of a compound/agent.

Conditions sufficient to induce resistance to insulin include, for example, the absence of insulin. Accordingly, an example of the invention provides for contacting a cell with insulin in the absence of serum for a time and under conditions to induce resistance to GLUT4 translocation. A cell that is resistant to insulin induced GLUT4 translocation is useful as a model for determining or identifying or isolating a modulator of insulin resistance, such as, for example, non-insulin dependent diabetes mellitus (NIDDM, type II diabetes).

Other methods for inducing resistance to translocation of a membrane transport protein will be apparent to those skilled in the art. For example, resistance to insulin induced translocation of a GLUT protein other than GLUT4 or a mutant thereof is induced using a method essentially as described sura.

Parallel Cellular Samples

One form of the present invention provides for performing the present invention in parallel cellular samples. Accordingly, the present invention provides a process for determining the level of a membrane transport protein translocated to the plasma membrane of a cell, said process comprising:
(a) determining the level of the membrane transport protein at the plasma membrane of a cell using a method comprising:
   (i) contacting a cell with a ligand that binds to the extracellular domain of the membrane transport protein for a time and under conditions sufficient for the ligand to bind the labeled membrane transport protein; and
   (ii) determining the level of ligand bound to the membrane transport protein;
(b) determining the level of membrane transport protein in another cell using a method comprising:
   (i) permeabilizing or disrupting the other cell;
   (ii) contacting the membrane transport protein with the ligand for a time and under conditions sufficient for the ligand to bind the membrane transport protein;
   (iii) determining the level of ligand bound to the membrane transport protein; and
(c) comparing the level of ligand detected at (a) (ii) and (b) (iii) to determine the level of the membrane transport protein at the plasma membrane relative to the total level of membrane transport protein.

As described supra, an example of the invention utilizes a labeled membrane transport protein to facilitate detection of the protein. Accordingly, the present invention provides a process for determining the level of a labeled membrane transport protein translocated to the plasma membrane of a cell, said process comprising:
(a) determining the level of the labeled membrane transport protein at the plasma membrane of a cell using a method comprising:
   (i) contacting a cell with a ligand that binds to the label for a time and under conditions sufficient for the ligand to bind the labeled membrane transport protein; and
   (ii) determining the level of ligand bound to the labeled membrane transport protein;
(b) determining the level of labeled membrane transport protein in another cell using a method comprising:
   (i) permeabilizing or disrupting the other cell;
   (ii) contacting the labeled membrane transport protein with a ligand that binds to the label for a time and under conditions sufficient for the ligand to bind the labeled membrane transport protein;
   (iii) determining the level of ligand bound to the labeled membrane transport protein; and
(c) comparing the level of ligand detected at (a) (ii) and (b) (iii) to determine the level of the labeled membrane transport protein at the plasma membrane relative to the total level of labeled membrane transport protein.

As used herein, the term “parallel cellular sample” shall be taken to mean that the cells used in the performance are grown under essentially or substantially the same conditions. Accordingly, cells are grown in, for example, the same or similar growth medium and/or grown at approximately the same temperature and/or grown in the same concentration of CO₂. Preferably, the cells are also isogenic.

As used herein, the term “isogenic” shall be taken to refer to cells that are derived from a clonal cell line. Accordingly, such cells are substantially identical at the genetic level. Preferably, each of the cells is from the same cell line.

For example, a cell that expresses a recombinant membrane transport protein preferably comprises an expression construct (encoding the recombinant membrane transport protein) that has stably integrated into the genome of the cell. Such stable integration means that cells derived from the original cell also comprise the expression construct and express the encoded protein. Furthermore, stable integration of the expression construct facilitates a standard or relatively unvarying level of expression of the membrane transport protein in cells derived from the original cell.

By culturing cells in parallel comparisons are made more reproducible. This is because variables controlled or influenced by the environment in which a cell is grown or cultured, such as, for example, gene expression levels are essentially controlled. Accordingly, a direct comparison between the level of a membrane transport protein at the cell surface of one cell compared to the level of a membrane transport protein in another (isogenic) cell cultured under essentially the same conditions facilitates determining the level of the membrane transport protein translocated to the plasma membrane as a function of the level of the membrane transport protein in the cell.

Methods for determining the level of a ligand bound to a membrane transport protein and/or the level of a membrane transport protein are described supra and are to be taken to apply mutais mutandis to the method for determining the level of a membrane transport protein translocated to the plasma membrane of a cell using a plurality of cells.

In one example, the process of the invention is performed in a plurality of cells. In accordance with this example, the inventive assay additionally comprises normalizing the determined level of ligand bound to the membrane transport protein with regard to the number of cells in which the level of the ligand bound to the membrane transport protein is determined. Methods for normalizing the determined level of ligand bound to the membrane transport protein are described supra.

Such normalization facilitates not only inter assay comparisons but also for determining the level of translocation of a membrane transport protein using cells cultured in, for example, parallel.

In an exemplified form of the invention, the inventors contacted a sample comprising cells with a labeled wheat germ agglutinin (WGA) for a time and under conditions sufficient for the WGA to bind to its ligand in the plasma membrane of a cell, and determining the level of WGA in the sample. For example, the sample is washed to remove any unbound WGA prior to detection. The level of WGA detected in the sample facilitates normalization of the level of the level of membrane transport protein detected relative to cell number. Clearly this facilitates determining the level of translocation of a membrane transport protein in addition to facilitating comparison between different samples.

Using the method of the present invention, the present inventors have produced a method for determining the level of a labeled GLUT4 protein or mutant thereof translocated to the plasma membrane of a cell. Accordingly, the present invention provides a process for determining the level of a labeled GLUT4 protein or labeled mutant GLUT4 protein translocated to the plasma membrane of a cell, said process comprising:
(a) determining the level of the labeled GLUT4 protein or labeled mutant GLUT4 protein at the plasma membrane of a cell expressing the labeled GLUT4 protein or labeled mutant GLUT4 protein using a method comprising:
   (i) contacting the cell with a ligand that binds to the label for a time and under conditions sufficient for the ligand to bind to the labeled GLUT4 protein or labeled mutant GLUT4 protein; and
   (ii) detecting the level of ligand bound to the labeled GLUT4 protein or labeled mutant GLUT4 protein;
(b) determining the level of membrane transport protein in another cell expressing the labeled GLUT4 protein or labeled mutant GLUT4 protein using a method comprising:
   (i) permeabilizing or disrupting the other cell;
   (ii) contacting the labeled GLUT4 protein or labeled mutant GLUT4 protein with a ligand that binds to the label for a time and under conditions sufficient for the ligand to bind to the labeled GLUT4 protein or labeled mutant GLUT4 protein;
   (iii) detecting the level of ligand bound to the labeled GLUT4 protein or labeled mutant GLUT4 protein; and
(c) comparing the level of ligand detected at (a) (ii) and (b) (iii) to determine the level of the labeled GLUT4 protein or labeled mutant GLUT4 protein at the plasma membrane relative to the total level of labeled GLUT4 protein or labeled mutant GLUT4 protein.

Furthermore, the present inventors have adapted this method to determine the level of a labeled GLUT4 protein or mutant thereof translocated to the plasma membrane of a cell that is resistant to insulin induced GLUT4 translocation. Accordingly, the present invention additionally provides a process for determining the level of the level of a labeled GLUT4 protein or a labeled mutant GLUT4 protein translocated to the plasma membrane of a cell that is resistant to insulin induced GLUT4 translocation, said process comprising:
(a) contacting a plurality of cells expressing a labeled GLUT4 protein or a labeled mutant GLUT4 protein with insulin for a time and under conditions sufficient to induce resistance to insulin induced GLUT4 translocation in the cell;
(b) determining the level of the labeled GLUT4 protein or labeled mutant GLUT4 protein at the plasma membrane of a cell (a) using a method comprising:
   (i) contacting the cell with a ligand that binds to the label for a time and under conditions sufficient for the ligand to bind to the labeled GLUT4 protein or labeled mutant GLUT4 protein; and
   (ii) detecting the level of ligand bound to the labeled GLUT4 protein or labeled mutant GLUT4 protein;
(c) determining the level of membrane transport protein in another cell (a) using a method comprising:
   (i) permeabilizing or disrupting the other cell;
   (ii) contacting the labeled GLUT4 protein or labeled mutant GLUT4 protein with a ligand that binds to the label for a time and under conditions sufficient for the ligand to bind to the labeled GLUT4 protein or labeled mutant GLUT4 protein;
   (iii) detecting the level of ligand bound to the labeled GLUT4 protein or labeled mutant GLUT4 protein; and
(d) comparing the level of ligand detected at (b) (ii) and (c) (iii) to determine the level of the labeled GLUT4 protein or labeled mutant GLUT4 protein at the plasma membrane relative to the total level of labeled GLUT4 protein or labeled mutant GLUT4 protein.

Methods for inducing resistance to GLUT4 translocation are described supra and are to be taken to apply mutatis mutandis to the instant example of the method of the invention.

As will be apparent to the skilled artisan the use of a labeled membrane transport protein is a model for the translocation of a wild-type or unlabeled membrane transport protein. For example, the label does not affect the function and/or translocation of the labeled membrane transport protein.

Determining Recycling of a Membrane Transport Protein

As a membrane transport protein is also recycled or turned-over from the plasma membrane of a cell (i.e. the membrane transport protein is removed from the membrane) the present invention additionally provides a method for determining the level or rate of recycling of a membrane transport protein in a cell. Accordingly, the present invention additionally provides A process for determining the level of recycling of a membrane transport in a cell comprising:
(a) determining the level of the membrane transport protein translocated to the plasma membrane of a cell using the process of the invention;
(b) determining the level of the membrane transport protein translocated to the plasma membrane of another cell using the process of the invention, wherein the other cell is cultured for a longer period of time than the cell (a); and
(c) comparing the level of the membrane transport protein translocated to the plasma membrane at (a) and (b) to determine the level of recycling of the membrane transport protein in the cell.

In another example, the present invention provides a process for determining a change in the level of recycling of a membrane transport in a cell comprising:
(a) determining the level of the membrane transport protein translocated to the plasma membrane of a cell using the process of the invention;
(b) determining the level of the membrane transport protein translocated to the plasma membrane of another cell using the process of the invention, wherein the other cell is cultured for a longer period of time than the cell (a); and
(c) comparing the level of the membrane transport protein translocated to the plasma membrane at (a) and (b),
   wherein a change in the level of the membrane transport protein translocated to the plasma membrane indicates a change in the level of recycling of a membrane transport protein.

As will be apparent to the skilled artisan an increase in the level of the membrane transport protein translocated to the plasma membrane at (b) compared to (a) is indicative of an enbalanced level of recycling of the membrane transport protein. In contrast, a reduction in the level of the membrane transport protein at (b) compared to (a) is indicative of an enhanced level of recycling of the membrane transport protein.

By determining the change in the level of the membrane transport protein at the plasma membrane at (a) and (b) and optionally expressing this as a function the rate of recycling of the membrane transport protein is determined. Clearly the present invention extends to determining the level of recycling of the membrane transport protein at a number of points in time and determining the rate of recycling of the membrane transport protein.

In one form of the invention, the cells are contacted with the ligand of the label throughout the process. The present inventors have shown that following binding of the ligand to the label, recycling of the membrane transport protein is not altered.

The methods described supra are also useful for determining the rate and/or level of internalization of a membrane transport protein. For example, a cell is incubated in the presence of an agent that induces translocation of the membrane transport protein to the plasma membrane and then the agent is removed. By determining the level of the membrane transport protein at the plasma membrane at a plurality of points of time following the removal of the agent the level and/or rate of internalization of the membrane transport protein is determined.

Accordingly, the present invention provides a method for determining the level of internalization of a membrane transport protein comprising:
(a) inducing translocation of a membrane transport protein by a method comprising contacting a plurality of cells with one or more peptides, polypeptides, proteins or compounds that induces translocation of the membrane transport protein for a time and under conditions for translocation to occur;
(b) determining the level of the membrane transport protein translocated to the plasma membrane of a cell (a) using the process of the invention;
(c) determining the level of the membrane transport protein translocated to the plasma membrane of another cell (a) using the process of the invention, wherein the other cell is cultured for a longer period of time than the cell (b); and
(d) comparing the level of the membrane transport protein translocated to the plasma membrane at (b) and (c),
   wherein the level of the membrane transport protein translocated to the plasma membrane at (b) compared to (c) indicates the level of internalization of the membrane transport protein.

Clearly this method applies mutatis mutandis to a method for determining the rate of internalization of a membrane transport protein.

Mutations Affecting Translocation of a Membrane Transport Protein

The process of the present invention is also useful for determining or identifying a mutation in a nucleic acid that encodes a membrane transport protein wherein the mutation affects the translocation of the membrane transport protein. Accordingly, the present invention provides a method for determining a mutation in a nucleic acid encoding a mutant membrane transport protein, wherein said mutation modulates translocation of said membrane transport protein, said method comprising:
(a) determining the level of the mutant membrane transport protein translocated to the plasma membrane of a cell using the process of the invention; and
(b) determining the level of a wild-type form of the membrane transport protein translocated to the plasma membrane of a cell using the process of the invention, wherein an enhanced or suppressed level of translocation of the membrane transport protein at (a) compared to (b) indicates that the nucleic acid comprises a mutation that modulates the level of level of translocation of the membrane transport protein to the plasma membrane.

As will be apparent to the skilled artisan, this method may also be adapted to determine the level of recycling or internalization essentially as described supra.

In one form of the invention both the mutant and wild-type form of the membrane transport protein are expressed in the same cell. As will be apparent to the skilled artisan, labeling each of the membrane transport proteins with a different label facilitates detection of each protein.

In another form of the invention, the mutant and wild-type form of the membrane transport protein are expressed in different cells. Accordingly, each membrane transport protein may be with the same label.

In one form of the invention, the process additionally comprises providing a cell expressing a mutant membrane transport protein and/or a wild-type form of the membrane transport protein. Methods for providing a cell, e.g. production of an expression construct and/or transforming/transfecting the expression construct into a cell are known in the art and described, for example, supra.

A mutant or mutated form of a membrane transport protein is isolated from a subject suffering from, for example, a disorder thought to be associated with aberrant translocation of a membrane transport protein.

Alternatively, or in addition, a mutant form of a membrane transport protein is produced using recombinant means. Means for producing a mutation in a nucleic acid are known in the art and include for example, site-directed mutagenesis or PCR mediated mutagenesis. Such methods are described for example, in Ausubel et al (In: Current Protocols in Molecular Biology. Wiley Interscience, ISBN 047 150338, 1987), Sambrook et al (In: Molecular Cloning: Molecular Cloning: A Laboratory Manual, Cold Spring Harbor Laboratories, New York, Third Edition 2001) or Dieffenbach (ed) and Dveksler (ed) (In: PCR Primer: A Laboratory Manual, Cold Spring Harbour Laboratories, NY, 1995).

The present inventors have produced various mutations in a cDNA encoding GLUT4 by, for example, site-directed mutagenesis or replacing regions of GLUT4 with regions from GLUT3. Furthermore, the present inventors have shown that these mutations affect the level of translocation of the mutant membrane transport protein.

In an example of the invention, the process additionally comprises determining the level of an expression product (e.g., mRNA or protein) encoded by the mutant and/or nucleic acid. Determining the level of expression of each nucleic acid facilitates comparing said expression levels to determine a compound that modulates the level of translocation of a membrane transport protein rather than modulating the level of expression of a membrane transport protein. Methods for determining expression levels are known in the art and/or are described, for example, in Ausubel et al (In: Current Protocols in Molecular Biology. Wiley Interscience, ISBN 047 150338, 1987), Sambrook et al (In: Molecular Cloning: Molecular Cloning: A Laboratory Manual, Cold Spring Harbor Laboratories, New York, Third Edition 2001) or Scopes (In: Protein purification: principles and practice, Third Edition, Springer Verlag, 1994).

Modulatory Agents

The present invention provides an assay that is easily amenable to a process for the identification of compounds that modulate the level of translocation of a membrane transport protein. For example, the present inventors have shown that the process of the invention may be performed in a 384 well format thereby facilitating high-throughput screening for a modulatory compound. Accordingly, the present invention additionally provides a process for determining an agent that modulates translocation of a membrane transport protein to the plasma membrane of a cell, said process comprising:
(a) determining the level of a membrane transport protein translocated to the plasma membrane of a cell in the absence of a candidate agent by performing the process of the invention; and
(b) determining the level of a membrane transport protein translocated to the plasma membrane of a cell in the presence of the candidate agent by performing the process of the invention,
   wherein a difference in the level of a membrane transport protein translocated to the plasma membrane of a cell at (b) compared to (a) indicates that the candidate agent modulates translocation of the membrane transport protein.

As will be apparent to the skilled artisan an agent that enhances the level of membrane transport protein at (b) compared to (a) enhances the level of translocation of the membrane transport protein. In contrast an agent that reduces the level of membrane transport protein at (b) compared to (a) reduces the level of translocation of the membrane transport protein

The agent may be derived from any source. For example, a test agent can be a pharmacologic agent already known in the art or can be an agent previously unknown to have any pharmacological activity. The agent can be naturally occurring or designed in the laboratory. The agent can be isolated from microorganisms, animals, or plants, or can be produced recombinantly, or synthesized by chemical methods known in the art. If desired, test compounds can be obtained using any of the numerous combinatorial library methods known in the art, including but not limited to, biological libraries, spatially addressable parallel solid phase or solution phase libraries, synthetic library methods requiring deconvolution, the “one-bead one-compound” library method, and synthetic library methods using affinity chromatography selection. The biological library approach is limited to polypeptide libraries, while the other four approaches are applicable to polypeptide, non-peptide oligomer, or small molecule libraries of compounds. See Lam, Anticancer Drug Des. 12, 145: 1997.

Methods for the synthesis of molecular libraries are known in the art (see, for example, DeWitt et al., Proc. Natl. Acad. Sci. U.S.A. 90: 6909, 1993; Erb et al. Proc. Natl. Acad. Sci. USA. 91:11422, 1994; Zuckermann et al., J. Med. Chem. 37: 2678, 1994; Cho et al., Science 261: 1303, 1993; Carell et al., Angew. Chem. Int. Ed. Engl. 33: 2059, 1994; Carell et al., Angew. Chem Int. Ed. Engl. 33: 2061; Gallop et al., J. Med. Chem. 37: 1233, 1994). Libraries of compounds are, for example, presented in solution (see, e.g., Houghten, Bio Techniques 13: 412-421, 1992), or on beads (Lam, Nature 354: 82-84, 1991), chips (Fodor, Nature 364: 555-556, 1993), bacteria or spores (Ladner, U.S. Pat. No. 5,223,409), plasmids (Cull et al., Proc. Natl. Acad. Sci. U.S.A. 89: 1865-1869, 1992), or:phage (Scott & Smith, Science 249: 386-390, 1990; Devlin, Science 249: 404-406, 1990); Cwirla et al., Proc. Natl. Acad. Sci. 97: 6378-6382, 1990; Felici, J. Mol. Biol. 222: 301-310, 1991; and Ladner, U.S. Pat. No. 5,223,409).

Alternatively, an agent is isolated from a natural compound library. Such a natural compound library is commercially available from, for example, InterBioscreen, Moscow, Russia.

The present inventors have shown that the fungal metabolite wortmanin is capable of suppressing GLUT4 translocation to the plasma membrane of a cell.

In one form of the invention a candidate agent is, for example an antibody or fragment thereof. Such an antibody is preferably capable of binding to and inhibiting the activity of a gene that is associated with or controls translocation of a membrane transport protein to the plasma membrane of a cell.

For example, the membrane transport protein is GLUT4 and the antibody binds to voltage-gated K+ channel, Kv1.3 thereby inhibiting the activity of the channel. Inhibition of the activity of this ion channel has been previously shown to enhance GLUT4 translocation to the plasma membrane.

In another form of the invention, the agent is an antisense nucleic acid, and RNAi molecule, a shRNA molecule or a ribozyme.

The term “antisense nucleic acid” shall be taken to mean DNA or RNA molecule that is complementary to at least a portion of a specific mRNA molecule (Weintraub, Scientific American 262:40, 1990) and capable of interfering with a post-transcriptional event such as mRNA translation. The use of antisense methods is known in the art (Marcus-Sakura, Anal. Biochem. 172: 289, 1988). Preferred antisense nucleic acid will comprise a nucleotide sequence that is complementary to at least 15 contiguous nucleotides of a sequence encoding the amino acid of the protein of interest.

As used herein, the term “ribozyme” shall be taken to refer to a nucleic acid molecule having nuclease activity for a specific nucleic acid sequence. To achieve specificity, preferred ribozymes will comprise a nucleotide sequence that is complementary to at least about 12-15 contiguous nucleotides of a sequence encoding a protein that modulates the translocation of a membrane transport protein.

As used herein, the terms “small interfering RNA” ('siRNA”), short hairpin RNA (“shRNA”), and “RNAi” refer to homologous double stranded RNA (dsRNA) that specifically targets a gene product, thereby resulting in a null or hypomorphic phenotype. Specifically, the dsRNA comprises two short nucleotide sequences derived from the target RNA and having self-complementarity such that they can anneal, and interfere with expression of a target gene, presumably at the post-transcriptional level. RNAi molecules are described by Fire et al., Nature 391: 806-811, 1998, and reviewed by Sharp, Genes & Development, 13: 139-141, 1999). As will be known to those skilled in the art, short hairpin RNA (“shRNA”) is similar to siRNA, however comprises a single strand of nucleic acid wherein the complementary sequences are separated an intervening hairpin loop such that, following introduction to a cell, it is processed by cleavage of the hairpin loop into siRNA. Accordingly, each and every embodiment described herein is equally applicable to siRNA and shRNA.

Preferred siRNA or shRNA molecules comprise a nucleotide sequence that is identical to about 19-21 contiguous nucleotides of the target mRNA. Preferably, the target sequence commences with the dinucleotide AA, comprises a GC-content of about 30-70% (preferably, 30-60%, more preferably 40-60% and more preferably about 45%-55%), and does not have a high percentage identity to any nucleotide sequence other than the target sequence in the genome of the animal in which it is to be introduced, e.g., as determined by standard BLAST search.

Methods for determining the level of translocation of a membrane transport protein are described supra and are taken to apply mutatis mutandis to the present method of the invention.

In one example, the method of the invention additionally comprises determining whether or not the agent is toxic. In accordance with this embodiment, the cells are screened to determine viability. Methods for determining viability include, for example, contacting a cell with a labeled agent that is incorporated or taken up by the cell for a time and under conditions sufficient for the cell to take up or incorporate the agent and detecting the label. Alternatively, the method comprises contacting a cell with a compound that is metabolized by the cell for a time and under conditions sufficient for the cell to metabolize the compound and detecting the metabolite.

For example, a cell viability assay comprises determining the level of ³H thymidine by a cell. Alternatively, trypan blue staining is useful for determining cell viability. Alternatively, or in addition, colorimetric assays such as for example, the ProCheck™ assay is available from Serologicals. A variety of other cell viability assays are known in the art and described for example, in Animal Cell Culture: Practical Approach, Third Edition (John R. W. Masters, ed., 2000), ISBN 0199637970.

For example, cell viability is measured using a methylthiazol tetrazolium (MTT) reduction assay (Mossman, J. Immunol. Meth., 65: 55, 1983). MTT is reduced by mitochondrial dehydrogenases in living cells; this reaction produces formazan crystals which are quantified by photometry after extraction. For example, using this method, an IC50 (concentration that reduces cell viability by 50%) is calculated.

Neutral red staining is also useful for determining cell viability. Neutral red is accumulated in the lysosomes in living cells that become colored by the dye. The dye is extracted and quantified using densitometry.

Alternatively, or in addition, cell viability is determined by determining the level of lactate dehydrogenase activity (Legrand et al., J. Biotechnol. 25:231-43, 1992). Lactate Dehydrogenase is a cytosolic enzyme that is released upon cell lysis. For example, an IC50 (concentration that reduces cell viability by 50%) can be calculated. This assay evidences chemicals inducing alterations in cell integrity (lysis). Kits for determining lactate dehydrogenase levels are commercially available from, for example, Promega or Vinci-Biochem, Vinci, Italy.

In one example, the present invention provides a process for determining an agent that modulates translocation of a membrane transport protein to the plasma membrane of a cell, said process comprising:
(a) determining the level of a membrane transport protein translocated to the plasma membrane of a cell in the absence of a candidate agent by performing
the process of the invention; (b) determining the level of a membrane transport protein translocated to the plasma membrane of a cell in the presence of the candidate agent by performing the process of the invention, wherein a difference in the level of a membrane transport protein translocated to the plasma membrane of a cell at (a) compared to (b) indicates that the candidate agent modulates translocation of the membrane transport protein.
(c) optionally, determining the structure of the candidate agent; and
(d) providing the candidate agent or the name or structure of the candidate agent.

Naturally, for agents that are known albeit not previously tested for their function using a screen provided by the present invention, determination of the structure of the compound is implicit in step (i) supra. This is because the skilled artisan will be aware of the name and/or structure of the compound at the time of performing the screen.

As used herein, the term “providing the agent” shall be taken to include any chemical or recombinant synthetic means for producing said agent or alternatively, the provision of an agent that has been previously synthesized by any person or means.

For example, a peptidyl compound is synthesized using is produced synthetically. Synthetic peptides are prepared using known techniques of solid phase, liquid phase, or peptide condensation, or any combination thereof, and can include natural and/or unnatural amino acids. Amino acids used for peptide synthesis may be standard Boc (Nα-amino protected Nα-t-butyloxycarbonyl) amino acid resin with the deprotecting, neutralization, coupling and wash protocols of the original solid phase procedure of Merrifield, J. Am. Chem. Soc., 85:2149-2154, 1963, or the base-labile Nα-amino protected 9-fluorenylmethoxycarbonyl (Fmoc) amino acids described by Carpino and Han, J. Org. Chem., 37:3403-3409, 1972. Both Fmoc and Boc Nα-amino protected amino acids can be obtained from various commercial sources, such as, for example, Fluka, Bachem, Advanced Chemtech, Sigma, Cambridge Research Biochemical, Bachem, or Peninsula Labs.

Synthetic peptides are alternatively produced using techniques known in the art and described, for example, in Stewart and Young (In: Solid Phase Synthesis, Second Edition, Pierce Chemical Co., Rockford, IIl. (1984) and/or Fields and Noble (Int. J. Pept. Protein Res., 35:161-214, 1990), or using automated synthesizers. Accordingly, peptides of the invention may comprise D-amino acids, a combination of D- and L-amino acids, and various unnatural amino acids (e.g., β-methyl amino acids, Cα-methyl amino acids, and Nα-methyl amino acids, etc) to convey special properties. Synthetic amino acids include ornithine for lysine, fluorophenylalanine for phenylalanine, and norleucine for leucine or isoleucine.

In another embodiment, a peptidyl agent is produced using recombinant means. For example, an oligonucleotide or other nucleic acid (eg., a nucleic acid encoding a dominant negative inhibitor of the protein of interest) is placed in operable connection with a promoter. Methods for producing such expression constructs, introducing an expression construct into a cell and expressing and/or purifying the expressed peptide, polypeptide or protein are known in the art and described supra.

Alternatively, the peptide, polypeptide or protein is expressed using a cell free system, such as, for example, the TNT system available from Promega. Such an in vitro translation system is useful for screening a peptide library by, for example, ribosome display, covalent display or mRNA display.

Methods for producing antibodies, preferably a monoclonal antibody, or a fragment or recombinant fragment thereof are described supra.

In a preferred embodiment, the compound or modulator or the name or structure of the compound or modulator is provided with an indication as to its use e.g., as determined by a screen described herein.

In another example, the invention provides a process for determining an agent that modulates translocation of a membrane transport protein to the plasma membrane of a cell, said process comprising:
(a) determining the level of a membrane transport protein translocated to the plasma membrane of a cell in the absence of a candidate agent by performing the process of the invention;
(b) determining the level of a membrane transport protein translocated to the plasma membrane of a cell in the presence of the candidate agent by performing the process of any one of the invention, wherein a difference in the level of a membrane transport protein translocated to the plasma membrane of a cell at (a) compared to (b) indicates that the candidate agent modulates translocation of the membrane transport protein.
(c) optionally, determining the structure of the candidate agent;
(d) optionally, providing the name or structure of the candidate agent; and
(d) providing, the candidate agent.

In one example, the candidate agent is provided with an indication as to its use, for example, as determined using a method described herein.

The present inventors have additionally produced a method for modeling insulin resistance. For example, the present inventors have produced a model in which a cell is resistant to insulin induced GLUT4 translocation. Accordingly, the present invention additionally provides a process for determining a candidate compound for the treatment of insulin resistance comprising:
(a) contacting a plurality of cells expressing a labeled GLUT4 protein or a labeled mutant GLUT4 protein with insulin for a time and under conditions sufficient to induce resistance to insulin induced GLUT4 translocation in the cell;
(b) determining the level of the labeled GLUT4 protein or a labeled mutant GLUT4 protein translocated to the plasma membrane of a cell (a) in the absence of a candidate agent by performing the process of the invention; and
(c) determining the level of the labeled GLUT4 protein or a labeled mutant GLUT4 protein translocated to the plasma membrane of another cell (a) in the presence of the candidate agent by performing the process of the invention,
   wherein a candidate agent that enhances the level -of translocation of the labeled GLUT4 protein or a labeled mutant GLUT4 protein is a candidate agent for the treatment of insulin resistance.

Conditions associated with insulin resistance include, for example, Syndrome X, type II diabetes (non-insulin dependent diabetes mellitus (NIDDM), hypertension, cardiovascular disease or obesity. Accordingly, an agent identified or determined using the method of the present invention is, for example, useful for the treatment of such a condition.

In one example, the agent is provided with an indication as to its use, for example, as determined using a method described herein.

The present invention additionally provides a process for determining a candidate compound for the treatment of insulin resistance comprising:
(a) contacting a plurality of cells expressing a labeled GLUT4 protein or a labeled mutant GLUT4 protein with insulin for a time and under conditions sufficient to induce resistance to insulin induced GLUT4 translocation in the cell;
(b) determining the level of the labeled GLUT4 protein or a labeled mutant GLUT4 protein translocated to the plasma membrane of a cell (a) in the absence of a candidate agent by performing the process of the invention;
(c) determining the level of the labeled GLUT4 protein or a labeled mutant GLUT4 protein translocated to the plasma membrane of another cell (a) in the presence of the candidate agent by performing the process of the invention, wherein a compound that enhances the level of translocation of the labeled GLUT4 protein or a labeled mutant GLUT4 protein is a candidate compound for the treatment of diabetes;
(d) optionally, determining the structure of the candidate agent; and
(e) providing the candidate agent or the name or structure of the candidate agent.

In one example, the agent is provided with an indication as to its use, for -example, as determined using a method described herein.

Furthermore, the present invention provides a process for determining a candidate compound for the treatment of insulin resistance comprising:
(a) contacting a plurality of cells expressing a labeled GLUT4 protein or a labeled mutant GLUT4 protein with insulin for a time and under conditions sufficient to induce resistance to insulin induced GLUT4 translocation in the cell;
(b) determining the level of the labeled GLUT4 protein or a labeled mutant GLUT4 protein translocated to the plasma membrane of a cell (a) in the absence of a candidate agent by performing the process of the invention;
(c) determining the level of the labeled GLUT4 protein or a labeled mutant GLUT4 protein translocated to the plasma membrane of another cell (a) in the presence of the candidate agent by performing the process of the invention, wherein a compound that enhances the level of translocation of the labeled GLUT4 protein or a labeled mutant GLUT4 protein is a candidate compound for the treatment of diabetes;
(d) optionally, determining the structure of the candidate agent;
(e) optionally, providing the name or structure of the candidate agent; and
(e) providing the candidate agent.

Suitable agents are known in the art and/or described supra.

Furthermore, methods for determining the level of translocation of a labeled GLUT4 protein or a labeled mutant GLUT4 protein translocated to the plasma membrane of a cell are known in the art and/or described herein.

For example, the method of the invention is useful for determining an agent for the treatment of diabetes, e.g., NIDDM.

Accordingly, the present invention additionally provides a process for manufacturing a medicament for the treatment of insulin resistance comprising:
(a) determining a candidate compound for the treatment of insulin resistance using a process comprising:
   (i) contacting a plurality of cells expressing a labeled GLUT4 protein or a labeled mutant GLUT4 protein with insulin for a time and under conditions sufficient to induce resistance to insulin induced GLUT4 translocation in the cell;
   (ii) determining the level of the labeled GLUT4 protein or a labeled mutant GLUT4 protein translocated to the plasma membrane of a cell (a) in the absence of a candidate agent by performing the process of the invention;
   (iii) determining the level of the labeled GLUT4 protein or a labeled mutant GLUT4 protein translocated to the plasma membrane of another cell (a) in the presence of the candidate agent by performing the process of the invention, wherein a compound that enhances the level of translocation of the labeled GLUT4 protein or a labeled mutant GLUT4 protein is a candidate compound for the treatment of diabetes;
(b) optionally, isolating the candidate agent;
(c) optionally, providing the name or structure of the candidate agent;
(d) optionally, providing the candidate agent; and
(e) using the candidate agent in the manufacture of a medicament for the treatment of insulin resistance.

Suitable agents and methods for determining their affect on GLUT4 translocation are described supra. Additionally, methods for inducing insulin resistance in a cell are described supra. For example, the cell is treated with insulin in the absence of serum for a time and under conditions sufficient to induce resistance to insulin induced GLUT4 translocation in the cell.

For example, the agent is formulated into a pharmaceutical formulation. Formulation of a pharmaceutical compound will vary according to the route of administration selected (e.g., solution, emulsion, capsule). An appropriate composition comprising the identified modulator to be administered can be prepared in a physiologically acceptable vehicle or carrier. For solutions or emulsions, suitable carriers include, for example, aqueous or alcoholic/aqueous solutions, emulsions or suspensions, including saline and buffered media. Parenteral vehicles can include sodium chloride solution, Ringer's dextrose, dextrose and sodium chloride, lactated Ringer's or fixed oils, for instance. Intravenous vehicles can include various additives, preservatives, or fluid, nutrient or electrolyte replenishers and the like (See, generally, Remington's Pharmaceutical Sciences, 17th Edition, Mack Publishing Co., Pa., 1985). For inhalation, the agent can be solubilized and loaded into a suitable dispenser for administration (e.g., an atomizer, nebulizer or pressurized aerosol dispenser).

Furthermore, where the agent is a protein or peptide or antibody or fragment thereof, the agent can be administered via in vivo expression of the recombinant protein. In vivo expression can be accomplished via somatic cell expression according to suitable methods (see, e.g. U.S. Pat. No. 5,399,346). In this embodiment, nucleic acid encoding the protein can be incorporated into a retroviral, adenoviral or other suitable vector (preferably, a replication deficient infectious vector) for delivery, or can be introduced into a transfected or transformed host cell capable of expressing the protein for delivery. In the latter embodiment, the cells can be implanted (alone or in a barrier device), injected or otherwise introduced in an amount effective to express the protein in a therapeutically effective amount.

The pH and exact concentration of the various components the formulation suitable for administration to the animal are adjusted according to routine skills in the art.

Following determination of an agent using a method described herein, the agent is additionally tested in vivo. For example, a candidate agent for the treatment of a mouse or rat model of NIDDM. For example, a mouse model is a mouse, such as for example a Cpe^{fat} mouse, a Lep^{ob} mouse, a Lepr^{ob} mouse or a tub mouse (all available from Jackson Laboratories). Alternative models of NIDDM include, for example, the tallyho mouse (Kim et al., Genomics 74: 273-286, 2001) or the OLETF rat (Watanabe et al., Genomics 58: 233-239). Such models are useful for, for example, determining the toxicity of a compound and/or the efficacy of a compound (e.g., the level or amount of the compound required for treatment).

The present invention is further described with reference to the following non-limiting examples

### EXAMPLE 1

### Generation and Expression of a labeled GLUT4 Protein

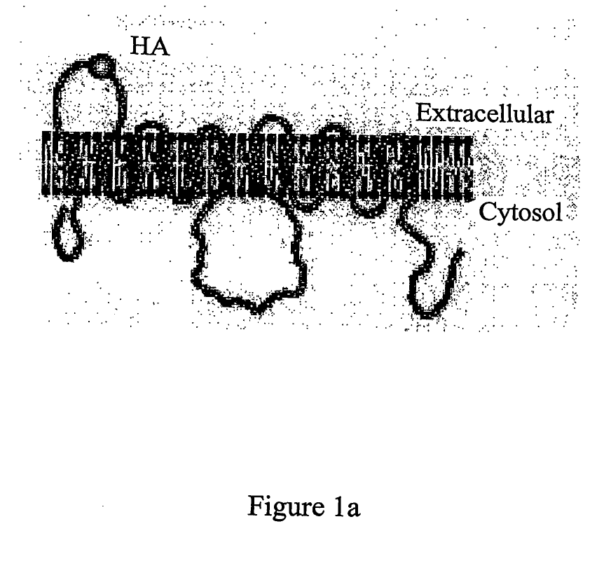

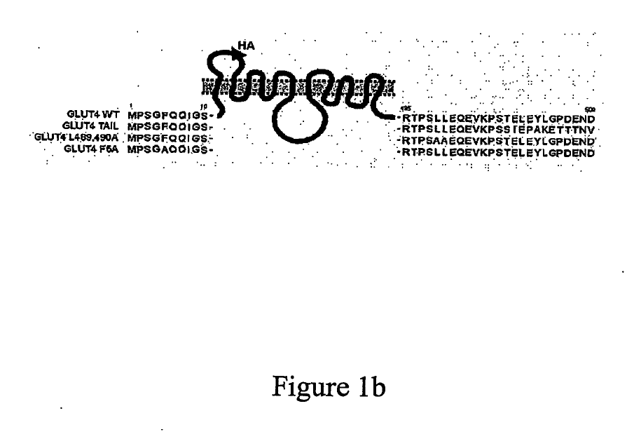

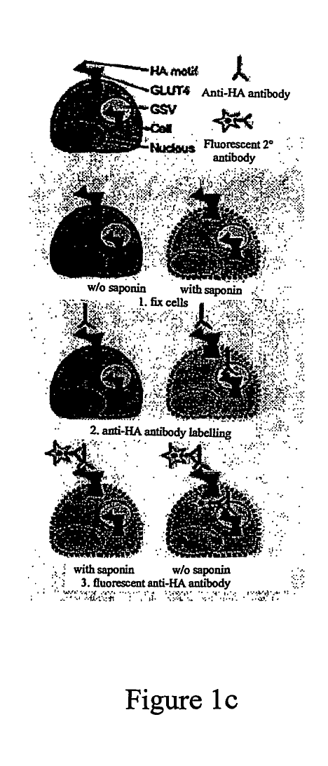

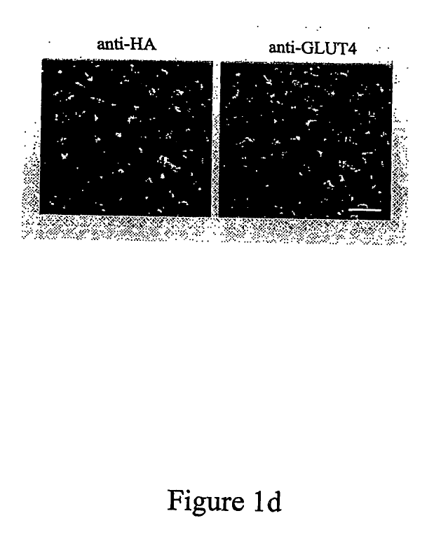

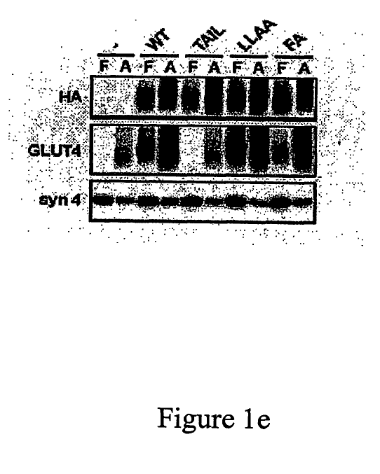

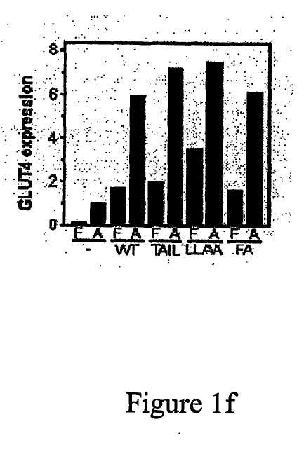

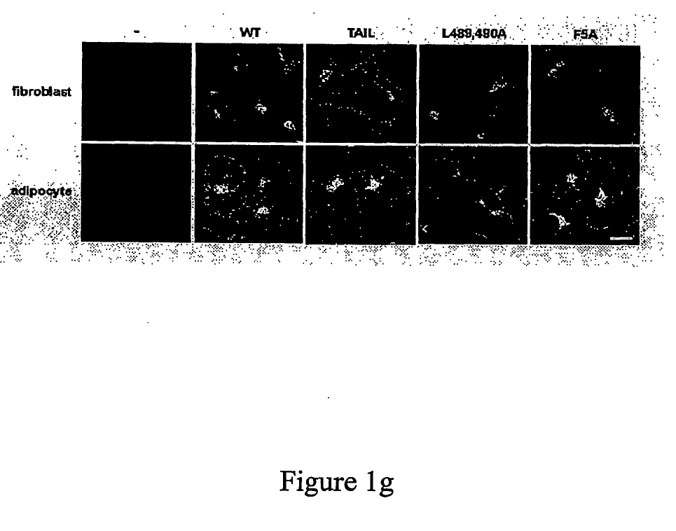

A HA-tagged GLUT4 protein was produced essentially as described in Quon et al., Proc. Natl. Acad. Sci USA 94: 5587-5591, 1994. Essentially, the cDNA encoding GLUT4 was digested with SauI and a double stranded oligonucleotide was inserted by ligation. The double stranded oligonucleotide was formed by hybridizing two oligonucleotides one comprising the sequence TGAGATCGATTATCCTTATGATGTTCCTGATTATGG (SEQ ID NO: 63) and the other TCA GCA TAA TCA GGA ACA TCA TAA GGA TAA TCG ATC (SEQ ID NO: 64). The inserted nucleic acid encodes a HA tag between amino acids 67 and 68 in the first exofacial loop of GLUT4 (SEQ ID NO: 4). This gene construct was inserted into the vector pBABE (Pear et al. Proc. Natl Acad Sci. U.S.A. 90: 8392-8396 1993). The polypeptide encoded by this protein is shown schematically in FIG. 1A.

Additional gene constructs were generated comprising a nucleic acid encoding mutant forms of GLUT4 (these constructs encoded the TALL mutant of GLUT4 (SEQ ID NO: 5), the L489,490A mutant of GLUT4 (SEQ ID NO: 7) and the F5A mutant of GLUT4 (SEQ NO: 9), each tagged with a HA tag), comprising a HA tag in the first extracellular domain of the protein, essentially as described in Piper et al, The Journal of Cell Biology, 121(6):1221-1232, 1993, Marsh et al, JCB, 130(5): 1081-1091, 1995, Shewan et al. Biochem. J. 350: 99-107, 2000 and Shewan et a, Mol Biol. Of Cell, 14: 973-986, 2003. The proteins encoded by these nucleic acids are schematically represented in FIG. 1B.

Retroviral stocks of each of the constructs were produced using the method described in Pear et al. Proc. Natl Acad. Sci. USA. 90: 8392-8396 1993. To generate 3T3-L1 adipocytes stably expressing the each construct 3T3-L1 fibroblasts (plated at a density of 5×10⁵/100 mm plate 16 h beforehand) were infected with the relevant virus for 3-5 h in the presence of 4 μg/ml Polybrene (Sigma). After a 48 h recovery period, infected cells were then selected in DMEM containing 10% FCS and supplemented with 2 μg/ml puromycin (Sigma).

3T3-L1 fibroblasts up to passage 20 were cultured in high glucose DMEM supplemented with 10% heat-inactivated new born calf serum (NCS) at 37° C. in 5% CO2. For differentiation into adipocytes, fibroblasts were cultured in DMEM/NCS for up to one or two days post-confluence, after which the cells were cultured for three days in DMEM containing 10% heat-inactivated fetal bovine serum (FBS), 350 nM insulin, 0.5 mM 3-isobutyl-1-methylxanthine (BMX), 250 nM dexamethasone, 400 nM biotin and for three days in DMEM containing 10% FBS and 350 nM insuin. After differentiation, adipocytes were maintained in DMEM supplemented with 10% FBS. Adipocytes were used for experiments 8 to 11 days after the onset of differentiation and the medium was renewed two or three days prior to each experiment. For culturing in gelatin-coated 96 well plates, cells were seeded at a 1:1 cell surface ratio and differentiation was initiated four days post-seeding.

To determine expression of the constructs transduced cells were studied suing immunofluorescence. Cells were stained for either the HA tag (Covance, Berkeley, Calif., USA) or anti-GLUT4 (Martin et al, J. Cell Biol. 134: 625-635, 1994). As shown in FIG. 1D approximately 90% of cells expressed the recombinant HA-GLUT4.

Steady state labeling of unstimulated cells revealed a predominant perinuclear GLUT4 localization in fibroblasts with low levels of GLUT4 in small peripheral vesicles. GLUT4 TAIL was more concentrated in peripheral vesicles compared to wild-type GLUT4 when expressed in fibroblasts (FIG. 1G).

Expression levels of the expression of the recombinant forms of GLUT4 was then determined using immunoblotting. Confluent 3T3-L1 fibroblasts and 3T3-L1 adipocytes at day 8 of differentiation were serum-starved for 2 h and lysed in PBS containing 1% Triton X-100, 1 mM EDTA, 1 mM PMSF, 10 μg/ml aprotinin and 10 μg/ml leupeptin. Equal amounts of protein were subjected to SDS-PAGE and transferred to PVDF membrane. Membranes were incubated with the indicated antibodies. HRP-conjugated secondary antibodies were visualized using ECL reagent (Pierce, Rockford, Ill.) and a 16 bit camera-based imager (VersaDoc 5000; Bio-Rad, Regents Park, Australia). For quantitation, a serial dilution of a control sample was run on the same SDS-PAGE gel and Quantity One software (Bio-Rad, Regents Park, Australia) was used for analysis. An anti-HA immunoblot was used to determine the relative expression of GLUT4 TAIL as this GLUT4 molecule was not recognized by the anti-GLUT4 antibody.

There was a modest level of overexpression (FIG. 1E and 1F), making it unlikely that GLUT4 localization was disturbed due to saturation of the cellular trafficking machinery.

### EXAMPLE 2

### Generation of an Assay to Determine the Localization of GLUT4

2.1 Methods

Retrovirally-transduced fibroblasts expressing HA-tagged GLUT 4 or a mutant therof were differentiated into adipocytes essentially as described above. These adipocytes were then subcultured for 30 hours. Insulin was then added at different time points, after which the cells were fixed in 3% formaldehyde. After washing and quenching with 50 mM glycine, cells were incubated for 20 min with 5% normal swine serum (NSS) in the absence or presence of 0.1% saponin to analyse the level of GLUT4 at the plasma membrane (PM) or the total cellular GLUT4 content, respectively. Cells were incubated for 60 min with a saturating concentration of either an antibody directed against the HA tag or a control non-relevant antibody (mouse IgG MOPC21) in PBS containing 2% NSS. After extensive washing, the cells were incubated for 20 min with 5% NSS in the presence or absence of 0.1% saponin to permeabilize all cells. Cells were incubated for 60 min with saturating concentrations of ALEXA488-conjugated goat-anti-mouse antibody (20 μg/ml) and ALEXA594-conjugated WGA (10 μg/ml) in PBS containing 2% NSS. After washing, fluorescence (emm 485/exc 520 and emm 544/exc 630) was measured using the bottom-reading mode in a fluorescence microtiter plate reader (FLUOstar Galaxy, BMG Labtechnologies, Offenburg, Germany). The percentage of GLUT4 at the PM was calculated for each condition. ALEXA594-WGA fluorescence was used to correct for variation in cell density in each well.

2.2 Results

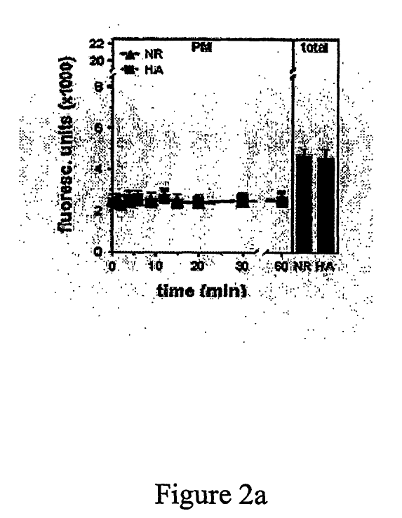

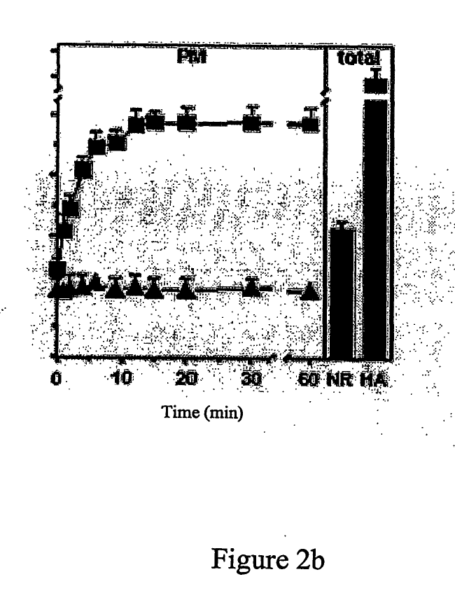

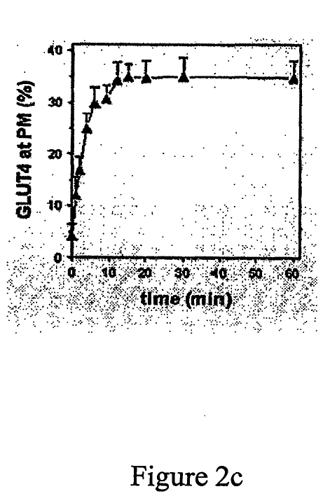

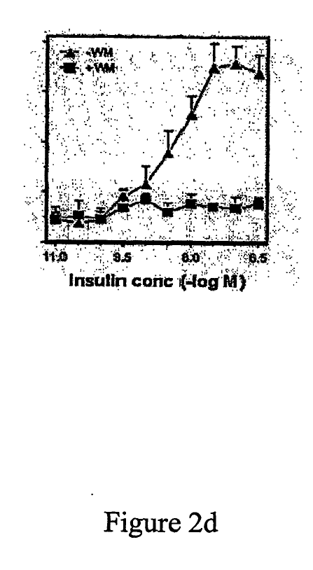

To determine the extent of insulin-induced GLUT4 translocation using the assay described supra, HA-GLUT4-expressing 3T3-L1 adipocytes grown in 96 well plates were incubated for 2 h in the absence of serum, whereafter 200 nM insulin was added at various time points and cell surface levels of HA-GLUT4 were analysed by indirect immunofluorescence labeling (FIG. 2B). Saturating levels of anti-HA and secondary antibodies were used to ensure that substantially all HA-GLUT4 molecules were labeled. A non-relevant antibody was used at the same concentration to determine the non-specific binding of the anti-HA antibody. Insulin stimulated the appearance of HA-GLUT4 at the PM with a half-time of about 2.5 min reaching a plateau by 12 min which was maintained for at least 60 min. No specific anti-HA labeling was detected in non-infected cells (FIG. 2A). Expressing the amount of specific fluorescence at the PM as a percentage of the total specific fluorescence revealed that insulin increased the level of GLUT4 at the PM from a basal value of 4% up to 34% (FIG. 2C) and this effect was inhibited by wortmannin (FIG. 2D).

### EXAMPLE 3

### Insulin Induced Translocation of GLUT4 in 3T3-L1 Fibroblasts and Adipocytes

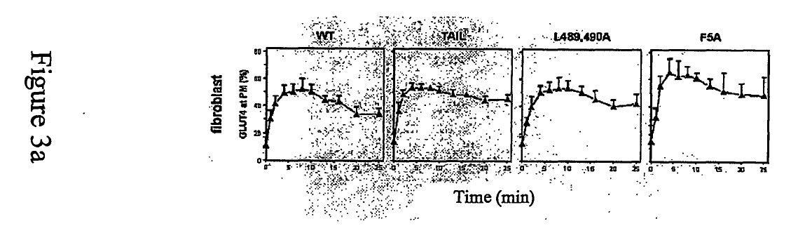

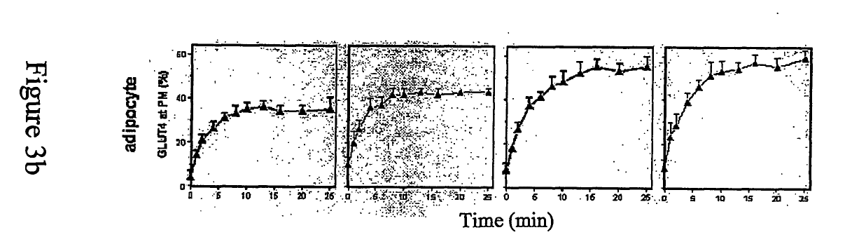

In fibroblasts, insulin induced the translocation of wild-type GLUT4 and each of the GLUT4 mutants to the PM (FIG. 3). The maximum level of surface GLUT4 was reached after 6 min of insulin stimulation, representing a 5-fold increase above that observed in non-stimulated cells, followed by a rapid reduction. The PM level of the GLUT4 F5A mutant was slightly higher than that of the other GLUT4 molecules in insulin-stimulated fibroblasts. In adipocytes we observed an ˜8-fold increase in cell surface GLUT4 levels in response to insulin stimulation. Neither wild-type GLUT4 nor any of the GLUT4 mutants showed an overshoot as was observed in fibroblasts. The GLUT4 TAIL mutant showed translocation characteristics similar to those of GLUT4 WT, although cell surface levels in both the absence and presence of insulin were increased by approximately 5%, in accordance with previous studies (Shewan et al., Mol. Biol. Cell 14: 973-986, 2003). The PM levels of both the L489,490A and F5A mutants were significantly higher than those of GLUT4 WT, both in the absence and presence of insulin.

### EXAMPLE 4

### GLUT4 Internalization and Recycling in 3T3-L1 Adipocytes

4.1 Methods

For single cycle internalization experiments cells were stimulated for 20 min with 200 nM insulin after starvation and washed on ice with ice-cold DMEM containing 20 mM HEPES pH 7.4 and 0.2% BSA. Cells were incubated with 100 nM wortmannin or 200 nM insulin and either anti-HA (25 μg/ml) or non-relevant antibody (MOPC21) in DMEM/HEPES/BSA for 1 h on ice. Wortmanin was added to abolish insulin signaling. This drug has no direct effect on GLUT4 internalization in adipocytes (Malide and Cushman J. Cell Sci. 110: 2795-2806) and has previously been used to study GLUT4 internalization (Al-Hasani et al, J. Biol. Chem. 273: 17504-17510). Cells were washed extensively, then either 100 nM wortmanin or 200 nM insulin in DMEM/HEPES/BSA was added. The plate was then transferred to 37° C. and at different times, formaldehyde was added to the wells to a concentration of 3%. After 5 min the formaldehyde was washed away and residual amounts were quenched. The cells were incubated for 20 min with 5% NSS in the absence of saponin, labeled with ALEXA488-conjugated goat-anti-mouse antibody and ALEXA594-conjugated WGA, washed and analysed as described above.

For continuous antibody uptake experiments, cells were incubated for 20 min with or without insulin, whereafter anti-HA (50 μg/ml) or non-relevant antibody was added. Cells that were used to determine the total amount of HA-GLUT4 were not incubated with antibody during this 37° C. incubation. After incubation, the cells were fixed and quenched as described above, and incubated for 20 min with 5% NSS and 0.1% saponin. Cells that were used to determine the total cellular amount of HA-GLUT4 were incubated for 60 min with anti-HA antibody or control antibody in PBS containing 2% NSS. All other cells were incubated with 2% NSS without antibody. Subsequently, the cells were incubated with ALEXA488-conjugated goat-anti-mouse antibody and ALEXA594-conjugated WGA, washed and analysed. The amount of specific anti-HA uptake was expressed as a percentage of total cellular immuno-reactive HA-GLUT4.

4.2 Analysis of GLUT4 Internalization in 3T3-L1 Adipocytes

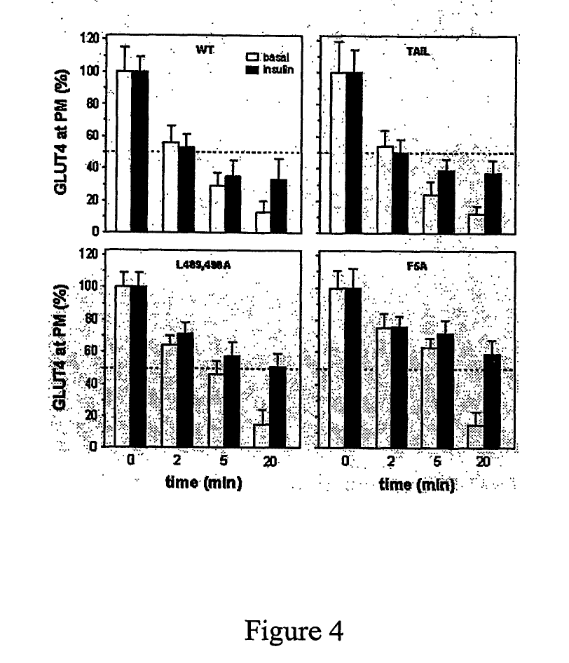

GLUT4 WT molecules that were labeled with anti-HA antibody on ice were rapidly cleared from the cell surface as indicated by the disappearance of GLUT4 at early time points after transfer of the cells from ice to 37° C. (FIG. 4). After approximately 5 min the level of GLUT4 at the PM reached steady state in the presence but not in the absence of insulin, indicating recycling of GLUT4 back to the PM in insulin-stimulated cells. Our data indicated that after 2 min at 37° C.˜50% of both GLUT4 WT and GLUT4 TAIL had disappeared from the PM. Importantly, this internalization rate was unaffected by insulin, consistent with previous studies (Satoh et al., J. Biol. Chem. 268: 17820-17829, 1993). The internalization rates for the L489,490A and ESA mutants were decreased by 30 and 45%, respectively (FIG. 4).

4.3 Anti-HA Antibody Uptake by HA-GLUT4-Expressing Fibroblasts and Adipocytes

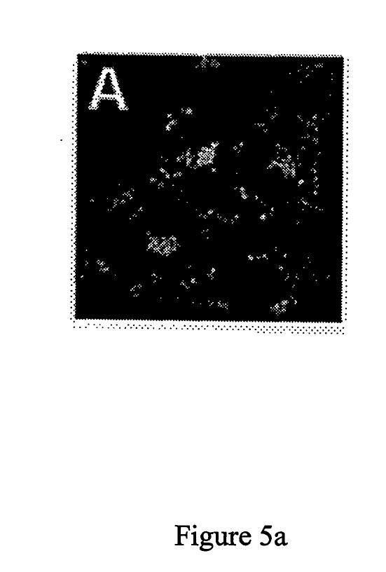

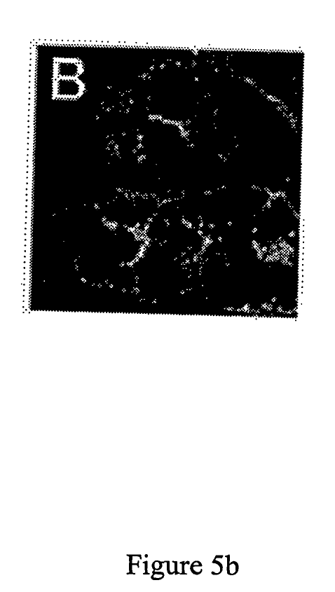

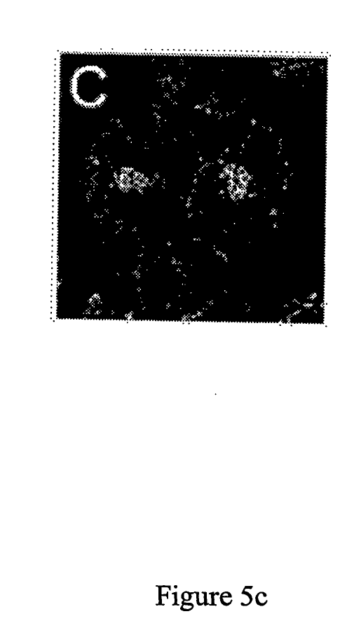

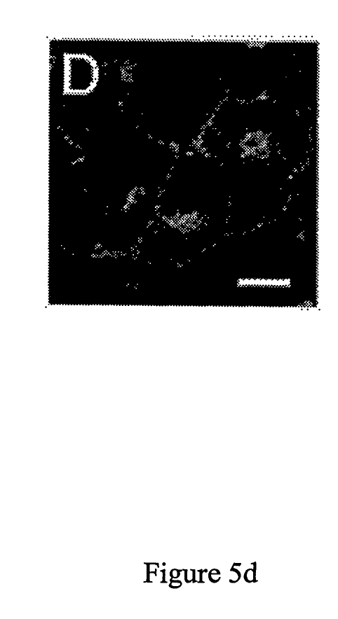

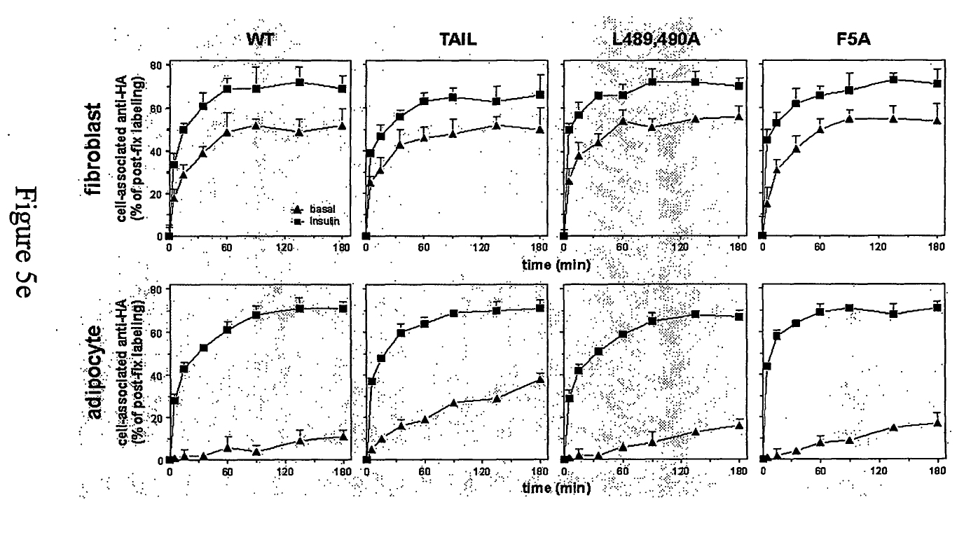

To analyze the exchange of GLUT4 with the cell surface under steady state conditions, studies were performed in which live cells were incubated with anti-HA antibody at 37° C. (FIG. 5). To ascertain that the anti-HA antibody did not affect the intracellular trafficking of HA-GLUT4, control experiments were performed in which insulin-induced translocation of anti-HA-bound HA-GLUT4 was studied. 3T3-L1 adipocytes expressing HA-GLUT4 WT were stimulated for 2 h with 200 nM insulin in the presence of anti-HA antibody, washed extensively, incubated for 2 h without insulin and anti-HA, and incubated for a further 20 min in the absence (FIG. 5C) or presence (FIG. 5D) of 200 nM insulin. The cells showed insulin-induced redistribution of anti-HA-bound HA-GLUT4 from intracellular compartments to the PM that was indistinguishable from translocation of HA-GLUT4 that had not been pre-labeled with antibody (FIG. 5A and 5B), indicating that the anti-HA antibody had no significant effect on GLUT4 trafficking.

For quantification of anti-HA antibody uptake, cells were preincubated for 20 min in the presence or absence of insulin after which anti-HA antibody or control antibody was added for various times (FIG. 5E). Antibody uptake was determined by labeling cells with fluorescent secondary antibody after fixation. Antibody uptake was expressed as a percentage of post-fixation anti-HA labeling.

Several observations were made from these studies. Firstly, there was a profound difference in recycling kinetics for HA-GLUT4 between fibroblasts and adipocytes in the absence of insulin. Whereas in fibroblasts a significant portion of the GLUT4 molecules recycled between intracellular compartments and the PM in the absence of insulin (˜50% after 60 min), this was not the case in adipocytes with only ˜10% of the entire GLUT4 pool labeled after 3 h. A similar percentage of GLUT4 was labeled after 6 h (not shown). Recycling of HA-GLUT4 in the presence of insulin was similar for fibroblasts and adipocytes. Secondly, the recycling rate of HA-GLUT4 TAIL in non-stimulated adipocytes was significantly higher than that observed for GLUT4 WT.

Thirdly, both of the internalization mutants showed a minor increase in basal anti-HA uptake and no difference in uptake during insulin stimulation compared with GLUT4 WT. Finally, it was noted that even with maximum insulin stimulation a small but significant pool of GLUT4 did not exchange with the cell surface under steady state conditions. The size of this pool was similar between fibroblasts and adipocytes and for the different GLUT4 mutants suggesting that it represents a pool of GLUT4 that is segregated from the insulin responsive pool.

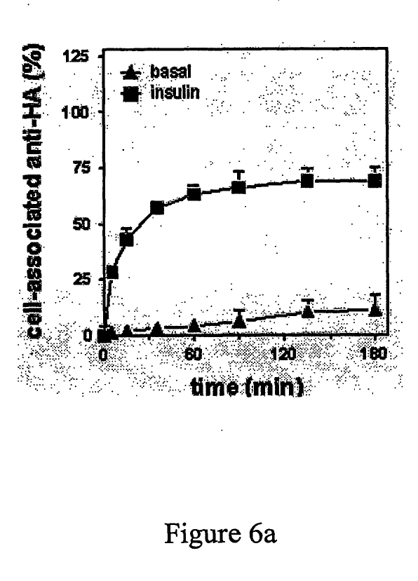

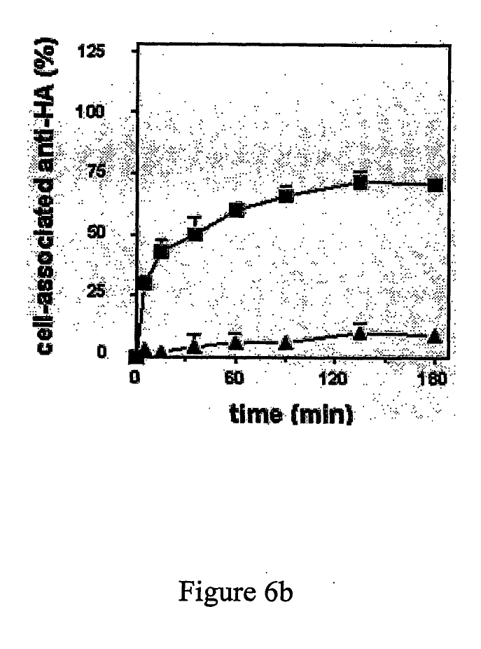

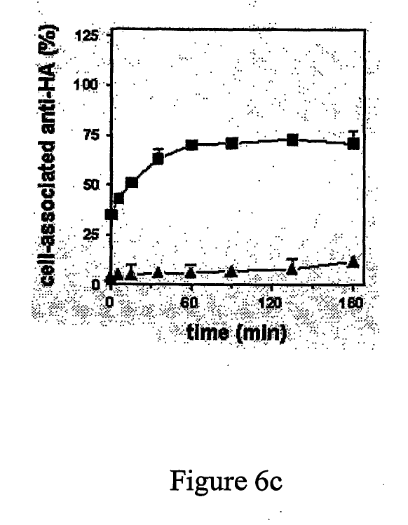

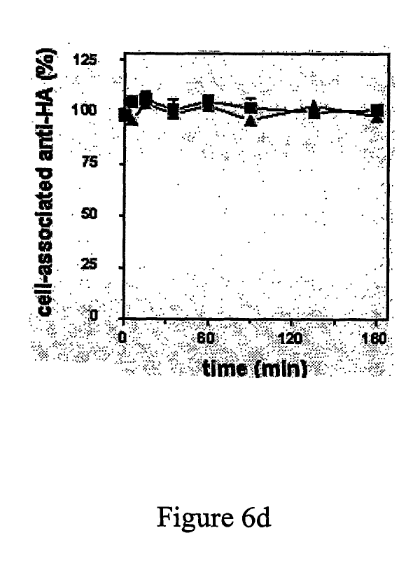

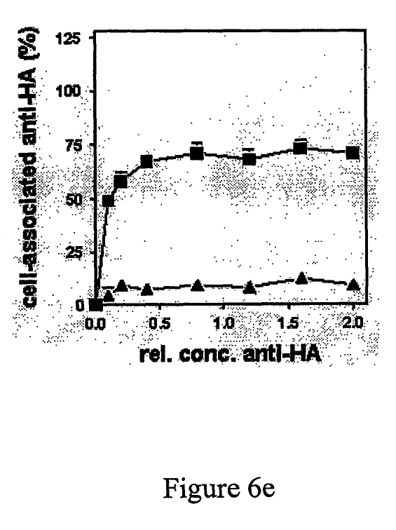

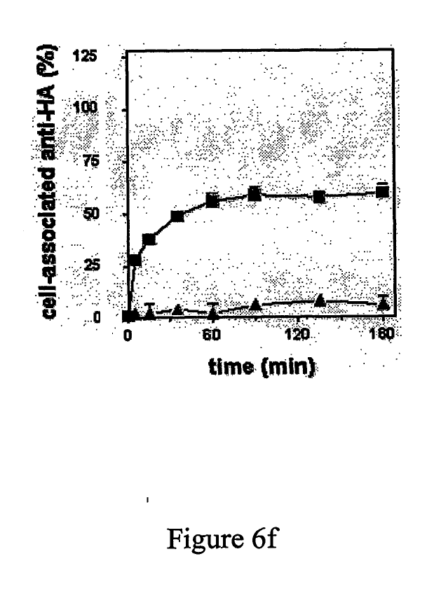

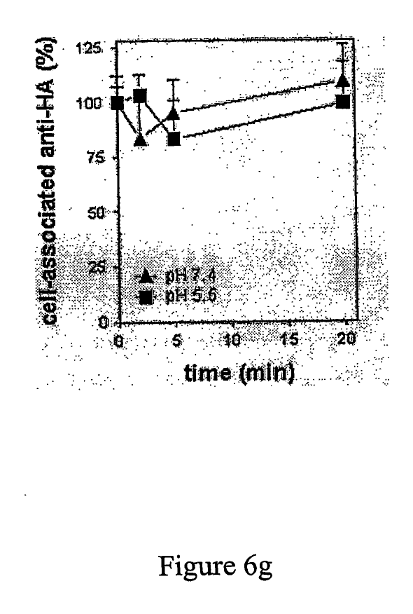

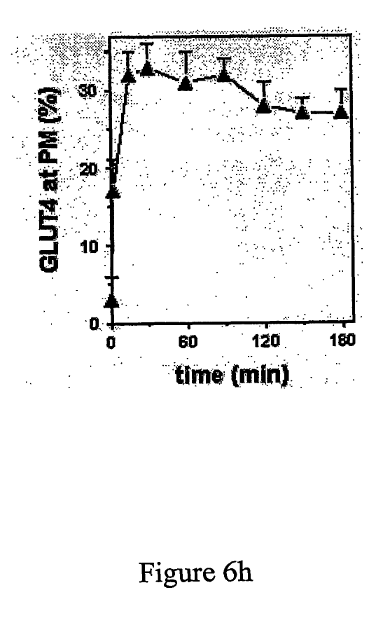

To study this non-recycling GLUT4 pool in adipocytes, 3T3-L1 adipocytes expressing HA-GLUT4 WT were incubated at 37° C. in the continuous presence of anti-HA antibody. Cells were incubated with or without 200 nM insulin for 20 min, after which anti-HA antibody was added in the continued presence or absence of insulin. Cells were incubated further for up to 180 min, fixed, permeablilized, and incubated with fluorescent secondary antibody. The level of anti-HA antibody taken up by the cells was then expressed as a percentage of total post-fixation anti-HA labeling of permeablilized cells. As shown in FIG. 6A, only approximately 30% of the HA-GLUT4 detected in a cell is labeled in the insulin induced cells. This suggests that approximately 30% of the HA-GLUT4 expressed in the cell did not translocate to the membrane during the experiment.

The cells that were used to determine the 100% value of HA-GLUT4 that recycled to the plasma membrane were incubated again with fixative after the post-fixation anti-HA immunolabeling. As shown in FIG. 6B fixation of the anti-HA antibody appeared not to change the affinity of the secondary antibody and therefore did appear not cause the 30% of difference in labeling.

Cells were again incubated with anti-HA after fixation without permeabilization. As shown in FIG. 6C the 30% of HA-GLUT4 that cannot be labeled with antibody during the 37° C. incubation is not present at the cell surface. Furthermore, cells were incubated again with the anti-HA antibody after fixation and permeabilization. In this case, 100% of GLUT4 was labeled, indicating that the 30% of HA-GLUT4 that cannot be labeled during the continuous antibody uptake is not unable to bind antibody but remains intracellular during the antibody uptake incubation.

To determine whether or not the antibody concentration used limited the level of HA-GLUT4 detected in a cell, cells were incubated for 3 h in the presence of insulin with various concentrations of anti-HA (in this regard, the standard concentration used was 50 mg/ml). As shown in FIG. 6E antibody concentration during the antibody incubation appeared not to be limiting with comparable levels of HA-GLUT4 being detected with various concentrations of anti-HA antibody.

To determine whether or not the unlabeled HA-GLUT4 was still in the process of synthesis or part of the biosynthetic tract cells were incubated with 10 mg/ml cycloheximide for 2 h prior to the addition of antibody. As shown in FIG. 6F 30% of GLUT4 could not be labeled, suggesting that the non-labeled GLUT4 pool is not part of the biosynthetic tract.

To determine the effect of endosomal pH on the binding of anti-HA antibody to HA-GLUT4 was determined. Cells were incubated for 30 min at 370C in hypertonic medium (0.45 M sucrose, pH 7.4), on ice with antibody in the same medium, and at 37° C. in hypertonic buffer at pH 7.4 or pH 5.5 in the absence of antibody. Release of antibody from the plasma membrane at neutral or endosomal pH was determined by incubating fixed non-permeablilized cells with fluorescent secondary antibody. As shown in FIG. 6G, endosomal pH did not induce the release of the anti-HA antibody from the HA-tag.

The effect of long-term insulin treatment on the amount of cell surface HA-GLUT4 levels was also determined. In this regard, cells were incubated for various times with 200 nM insulin and cell surface GLUT4 levels were determined as described supra. As shown in FIG. 6H, insulin did not drastically down-regulate cell surface GLUT4 levels, indicating that insulin-induced down-regulation of GLUT4 at the PM did not account for the limited HA-GLUT4 labeling during the continuous antibody uptake.

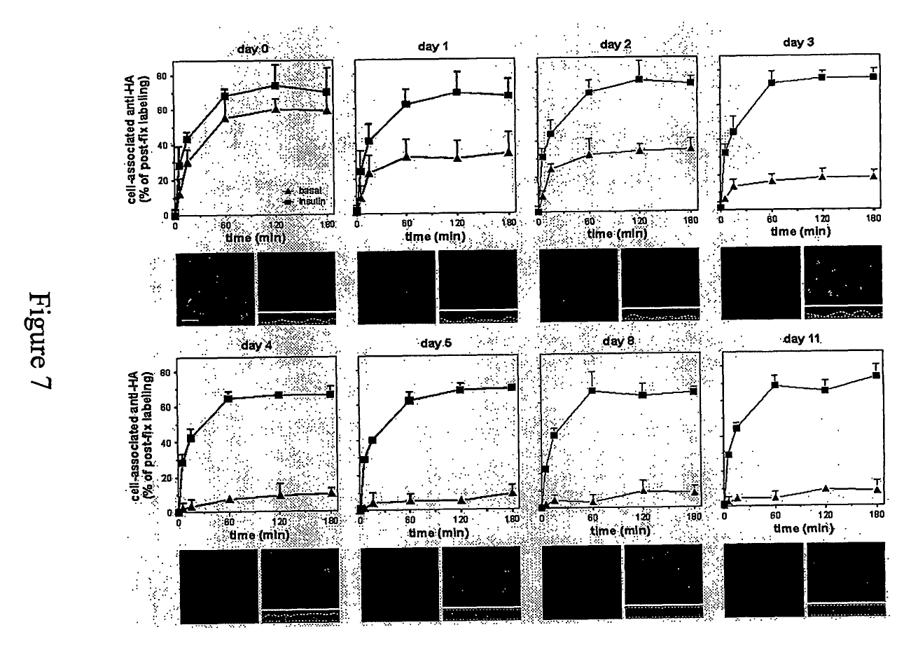

The recycling linetics of HA-GLUT4 was studied at different stages throughout fibroblast differentiation (FIG. 7). In parallel, antibody uptake was analysed by immunofluorescence confocal microscopy (FIG. 7, left microscopy panels) as well as endogenous GLUT4 labeling and lipid droplet content in non-infected cells (FIG. 7, right microscopy panels).

There was a progressive decline in antibody uptake between days 0 and 4 of differentiation. Expression of endogenous GLUT4 and lipid droplet formation were initially detected at day 3 when antibody uptake by non-stimulated cells had already decreased by 85% (compared with 100% at day 4). The final reduction in basal anti-HA uptake, between day 3 and 4, coincided with a massive growth of the cells (FIG. 7, right bottom microscopy panels).

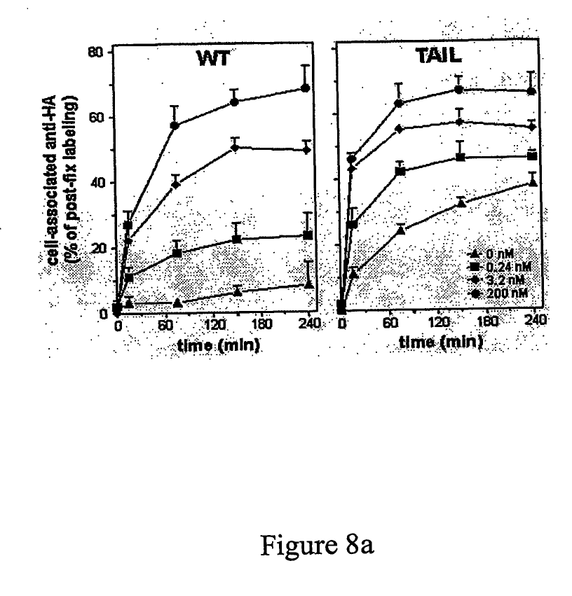

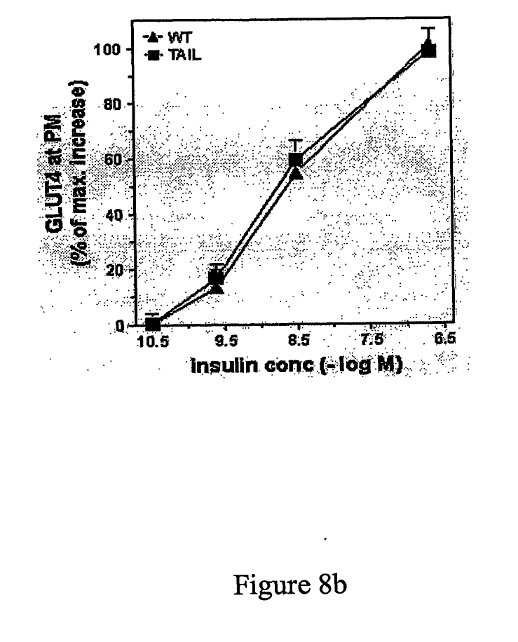

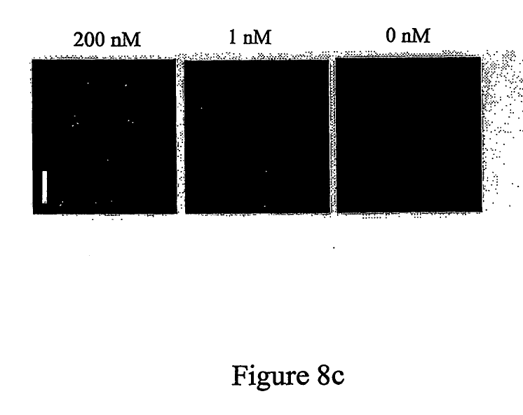

The results attained suggest that only part of the intracellular GLUT4 pool may be released into the cell surface recycling system as opposed to reduced trafficking kinetics of the entire intracellular GLUT4 pool. To test this recycling studies were performed at different doses of insulin (FIG. 8). These studies revealed that the size of the recycling pool of GLUT4 was incrementally increased with increasing doses of insulin.

This phenomenon was evident for both GLUT4 WT and GLUT4 TAIL, although insulin had a less profound effect on GLUT4 TAIL due to its elevated levels in the recycling pathway in the basal state (FIG. 5 and 8B). Measurement of cell surface levels of HA-GLUT4 at the different insulin doses revealed that the insulin dose response curves for translocation of both GLUT4 WT and TAIL were similar, despite major differences in their basal recycling properties (FIG. 8B).

To rule out the possibility that this incremental effect of insulin on entry of GLUT4 into the cell surface recycling system might reflect intrinsic differences in insulin sensitivity between individual cells within the culture the dose response relationship in antibody uptake in individual cells using immunofluorescence microscopy was examined. As indicated in FIG. 8C the response among different cells was highly homogeneous such that at low doses of insulin most cells exhibited a low level of antibody uptake and at higher doses there was a uniform rather than a heterogeneous increase in antibody uptake.

### EXAMPLE 5

### Development of a High-Throughput Assay for Determining GLUT4 Translocation

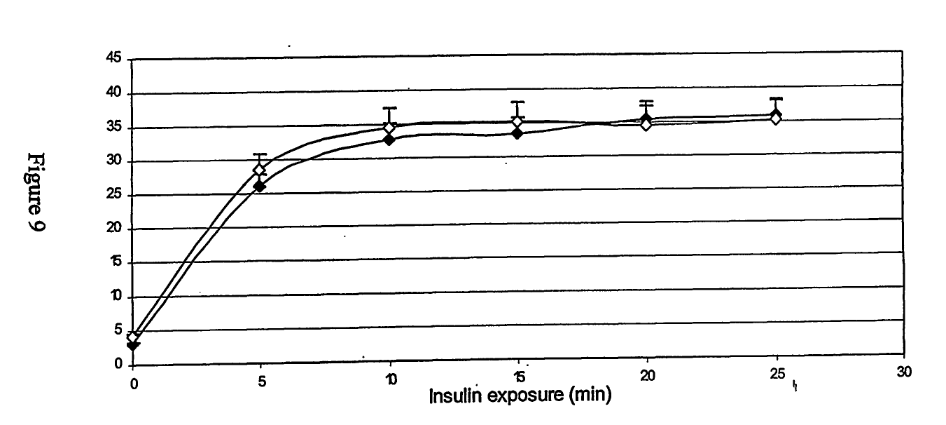

To determine the efficacy of a high throughput assay for analysing the level of translocation of a labeled membrane transport protein HA-GLUT4 expressing 3T3-L1 adipocytes were grown in 384 well plates or first grown in Petri dishes and then relocated into the 384 well plates. An incubation period of 2 hours was observed after which 200 nM insulin exposure was used for the indicated periods of time. For each time point the percentage of labeled GLUT4 (compared to the level of labeled GLUT4 following cell permeabilization) at the plasma membrane was calculated. As shown in FIG. 9 approximately equal levels of GLUT4 translocation was observed in both sample types. Accordingly, these results show the efficacy of a 384 high-throughput method for analysing GLUT4 translocation.

### EXAMPLE 6

### The Effect of Amino Acid Concentration on GLUT4 Translocation

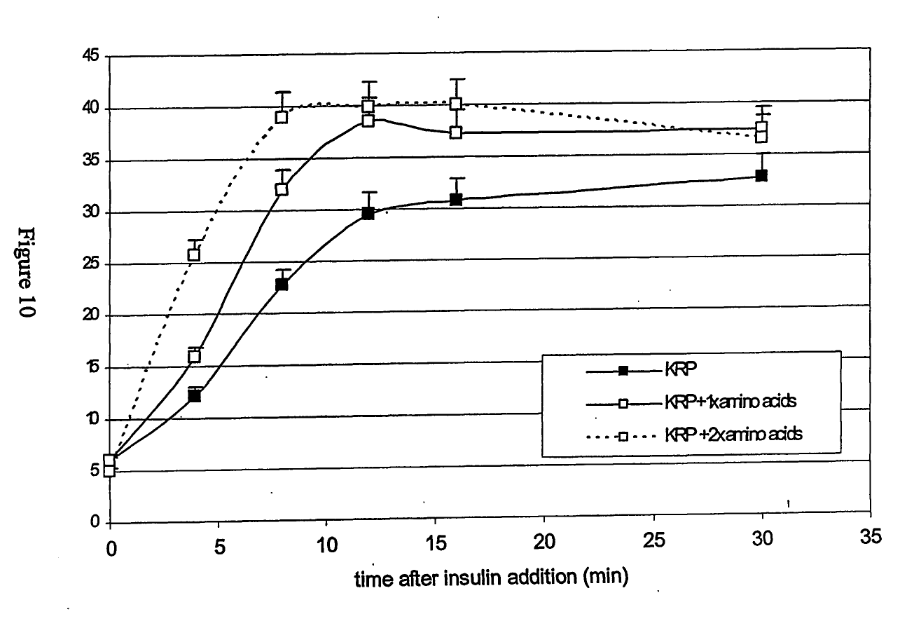

HA-GLUT4 expressing adipocytes were serum starved for 2 hours in Krebs Ringer Phosphate (KRP) buffer or in the same buffer supplemented with amino acid concentrations used in Dulbecco's modified eagle medium of Gibco (2× amino acids) or with half of the amino acid concentration (1× amino acids) respectively. Cells were then stimulated with 200 nM insulin essentially as described above and the percentage of HA-GLUT4 WT translocated to the membrane determined as described supra. As shown in FIG. 10 the concentration of amino acids in the medium in which cells were incubated influenced the level of GLUT4 translocated to the plasma membrane.

### EXAMPLE 7

### Inducing GLUT4 Translocation to the Plasma Membrane

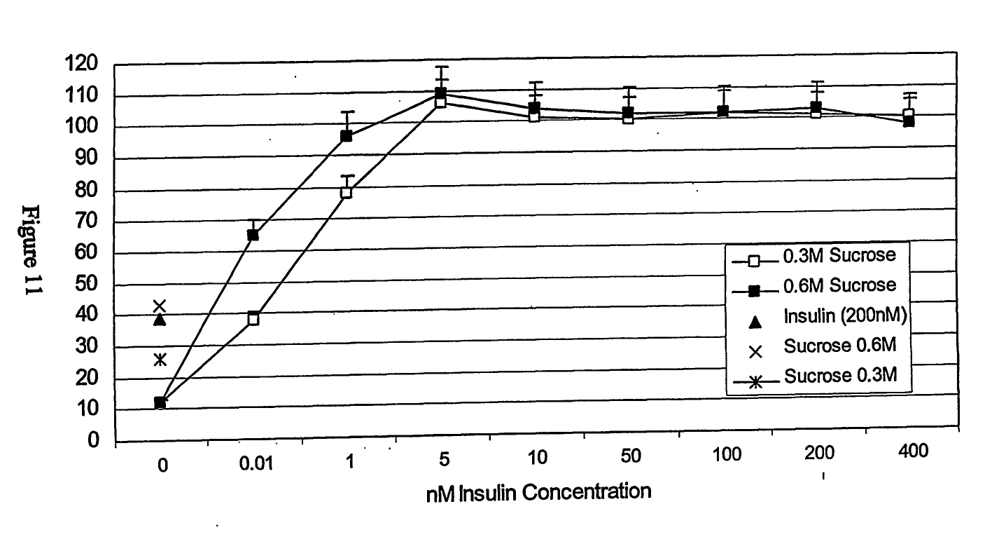

3T3-L1 adipocytes expressing HA-GLUT4 WT were serum starved for 2 hours at 37° C. Following 20 minutes insulin stimulation with 200 nM insulin, cells were incubated for additional 2 hours in serum free medium supplemented with 0.2% BSA and 0.3 or 0.6M sucrose. After post-fixation anti-HA immunolabeling the level of cell surface HA-GLUT4 levels was determined as a percentage of total HA-GLUT4 detected after cell lysis. As shown in FIG. 11, sucrose dramatically increases the level of HA-GLUT4 translocated to the plasma membrane of a cell. Furthermore, increasing concentrations of sucrose induce more GLUT4 to translocate to the plasma membrane in the presence of reduced levels of insulin.

### EXAMPLE 8

### Development of a Model of Insulin Resistance

3T3-L1 adipocytes retrovirally infected with GLUT4 (described in Example 1) were incubated 24 hours or 48 hours either with 600 nM insulin or with medium alone. After this chronic insulin stimulation (as indicated in FIG. 10) at 37° C. in a CO₂ incubator, cells were washed and 200 nM insulin was added for additional 10 or 30 minutes. Cell surface levels of HA-GLUT4 were measured using the fluorescence based assay described supra and expressed as a percentage of total HA-GLUT4 detected in the cell. The experiment was also performed with the HA-GLUT4 TAIL mutant

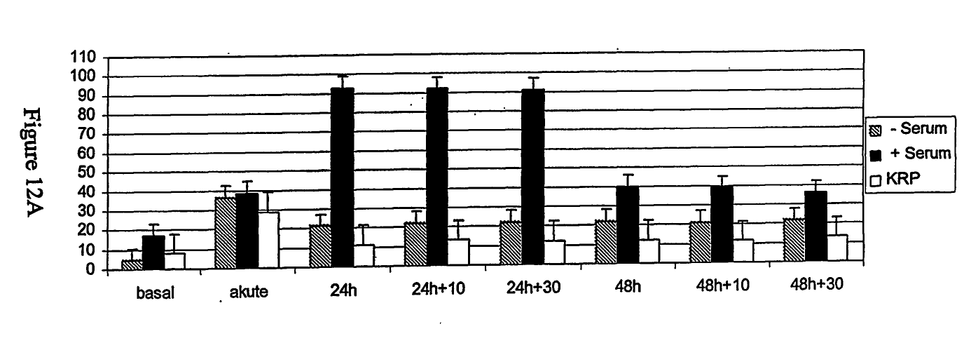

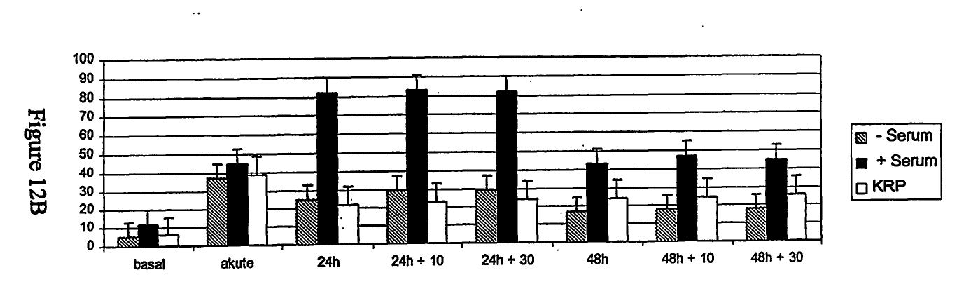

As shown in FIG. 12A the level of GLUT4 at the plasma membrane of cells incubated in the presence of serum was dramatically increased following 24 h incubation in the presence of insulin. However, this effect was suppressed following 48 h incubation in the presence of insulin.

A dramatically different effect was observed in cells incubated in the absence of serum (either -serum or KRP). The levels of GLUT4 translocation observed were little more than basal levels (i.e. cells in the absence of insulin). These results indicate that the cells were resistant to insulin induced GLUT 4 translocation. This assay represents an attractive model of insulin resistance for, for example, screening for agents for treating disorders characterised by insulin resistance.

As shown in FIG. 12B similar results were attained with the HA-GLUT4 TAIL mutant.

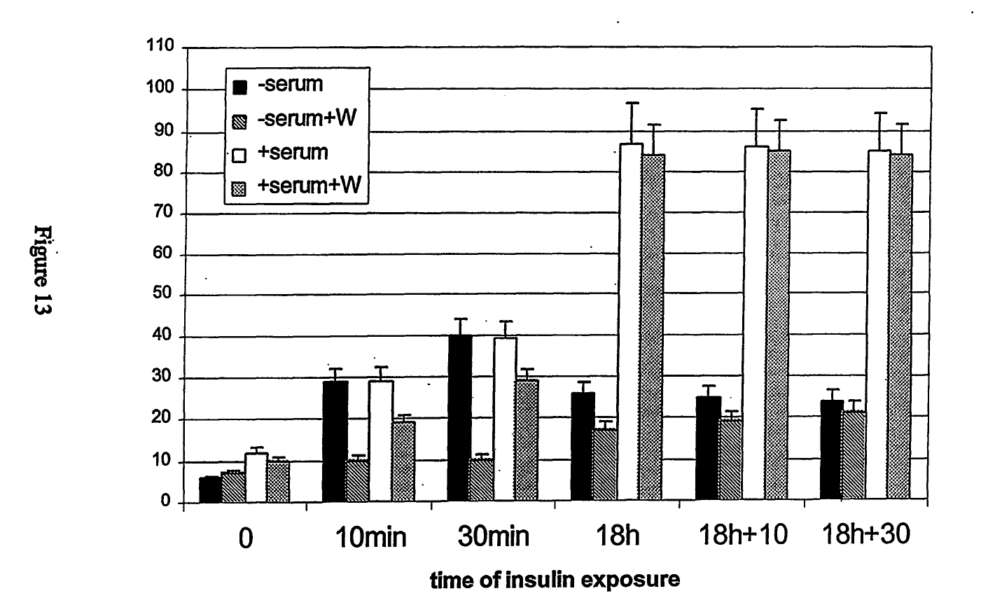

Furthermore, as shown in FIG. 13 wortmanin was shown to have little effect on the translocation of HA-GLUT4 in the presence of serum either following an acute or chronic exposure to insulin. HA-GLUT4 expressing 3T3-L1 adipocytes were grown in 96 well plates, incubated for 2 hours or overnight in medium supplemented with 10% fetal calf serum or no serum. 200 nM insulin in case of acute stimulation and 600 nM insulin in case of chronic stimulation have been used. After overnight stimulation cells were washed and 200 nM fresh insulin was added for 10 or 30 mln.

However, following an acute exposure to insulin, wortmnannin was able to reduce levels of HA-GLUT4 translocation in cells incubated in the absence of insulin. Following a chronic exposure of the cells to insulin wortmanin did not appear to significantly alter the levels of GLUT4 translocated to the plasma membrane.

### EXAMPLE 9

### Screening a Natural Product Library to Determine an Enhancer of GLUT4 Translocation

HA-GLUT4 expressing 3T3-L1 adipocytes are grown in 384 well plates essentially as described in Example 5. Cells are then incubated 24 hours with 600 nM insulin in the absence of serum. After this chronic insul stimulation at 37° C. in a CO₂ incubator cells are incubated in the presence of a compound from a natural product library, such as, for example, the plant extract library from TimTec (Newark, USA). 200 nM insulin is then added for an additional 10 or 30 minutes to each well. Cell surface levels of HA-GLUT4 is measured using the fluorescence based assay described supra and expressed as a percentage of total HA-GLUT4 detected in the cell. Results are also normalized for cell number using WGA, essentially as described in Example 2.

Samples are analysed to determine those natural products that are capable of inducing HA-GLUT4 translocation to the plasma membrane to a degree similar to that observed in a cell incubated in the presence of both serum and insulin (i.e. a positive control).

Cells cultured in parallel are also assayed using trypan blue exclusion to determine those natural products that are toxic to cells. Following incubation of the cells in the presence or absence (control) of the natural products, cells are treated with 1% trypan blue. The number of cells that have taken up the trypan blue stain in each treatment group is expressed as a percentage of the number of cells that have taken up the trypan blue stain in the control samples. Those compounds that significantly reduce the number of viable cells are considered to be at least partially toxic to a cell.

Compounds that enhance GLUT4 translocation without significantly reducing viability are then assessed using the assays supra to determine the concentration at which translocation is maximally enhanced without affecting cell viability.

### EXAMPLE 10

### In vivo Analysis of an Enhancer of GLUT4 Translocation

Male C57BL/KS-Lepdb (db/db) and nondiabetic littermate mice (The Jackson Laboratory) are obtained at 7-8 weeks of age and housed in 12 hr of light per day at 21-23° C. and 40-60% humidity. All experiments begin at 10 weeks of age. A compound determined in Example 9 is administered by sub cutaneous injection. For glucose tolerance testing, all animals were fasted for 16-18 hr before gavaging with a standard glucose bolus, as outlined Tonra et al., Diabetes 48: 588-594, 1999. Animals are then anesthetized and a bolus of insulin (1 unit) administered through the jugular vein; 2 or 10 min later, the liver is rapidly removed and frozen at −80° C. until processed.

Serum samples are taken between 1000 and 1200 hours and analyzed for glucose, triglycerides, and cholesterol with the Monarch blood chemistry analyzer (Instrumentation Laboratory, Lexington, Mass.). NEFA are analyzed with a diagnostic kit (Wako Chemical, Osaka) and insulin levels by ELISA (Linco Research Immunoassay, St. Charles, Mo.). For analysis of endogenous lipids, frozen sections of liver are mounted on glass slides and stained with oil red O. Liver glycogen is measured from frozen tissue by assaying for glucose after amyloglucosidase digestion with a correction for nonglycogen glucose (Tonra et al., Diabetes 48: 588-594, 1999).

Using these assays, mice are then assessed to determine hyperinsulinemia, hyperglycemia and glucose tolerance essentially as described in Sleeman et al., Proc Natl Acad Sci USA. 100:14297-14302, 2003. For example, serum glucose and insulin levels are determined.

### EXAMPLE 11

### An Assay to Determine a Suppressor of GLUT4 Translocation

HA-GLUT4 expressing 3T3-L1 adipocytes are grown in 384 well plates essentially as described in Example 5. Cells are then incubated with a compound from the natural product library supra and then 200 nM insulin. The level of HA-GLUT4 translocated to the palsma membrane is then measured.

Briefly, cells are fixed in 3% formaldehyde. After quenching with 50 mM glycine, cells are incubated for 20 min with 5% normal swine serum (NSS) in the absence or presence of 0.1% saponin to analyse the level of GLUT4 at the plasma membrane (PM) or the total cellular GLUT4 content, respectively. Cells are incubated for 60 min with a saturating concentration of either an antibody directed against the HA tag or a control non-relevant antibody (mouse IgG MOPC21) in PBS containing 2% NSS. After extensive washing, the cells are incubated for 20 min with 5% NSS in the presence or absence of 0.1% saponin to permeabilize all cells. Cells are incubated for 60 min with saturating concentrations of ALEXA488-conjugated goat-anti-mouse antibody (20 eg/ml) and ALEXA594-conjugated WGA (10 μg/ml) in PBS containing 2% NSS. After washing, fluorescence (emm 485/exc 520 and emm 544/exc 630) is measured using the bottom-reading mode in a fluorescence microtiter plate reader (FLUOstar Galaxy, BMG Labtechnologies, Offenburg, Germany). The percentage of GLUT4 at the PM is calculated for each compound. ALEXA594-WGA fluorescence was used to correct for variation in cell density in each well.

As a positive control the K+/H+ exchanger, nigericin, is used. Nigericin is known to inhibit insulin mediated GLUT4 translocation Chu et al., J Cell Biochem. 2002;85:83-91. The level of translocation of HA-GLUT4 for each natural compound is compared to that for nigericin and compounds with equal or greater inhibitory activity are selected.

In parallel cultures, the toxicity of each of the natural products is also assessed. Cell viability for each of the compounds tested is assessed using the CellTiter-Glo® Luminescent Cell Viability Assay (Promega) essentially according to manufacturer's instructions. Compounds that do not significantly reduce cell viability are selected for further analysis.

The compounds selected are then screened using the HA-GLUT4 translocation assay and the CellTiter-Glo® Luminescent Cell Viability Assay to determine the concentration at which each compound shows maximum activity without significantly reducing cell viability.

### EXAMPLE 12

### A Model for GLUT1 Translocation

12.1 Vector Construction

A human GLUT1 cDNA containing an Hemaglutinin epitope tag in its first exofacial loop was kindly provided in the pCIS2 expression vector by the Al-Hasani Lab.

HA-GLUT1 is then excised from this pCIS2 vector by NdeI and KpnI digestion and subcloned into the pOK12 plasmid . Following digestion with NdeI and KpnI, this reporter GLUT1 gene tagged with HA is then excised from pOK12 plasmid as a 1.8 kb ClaI/XbaI fragment and subcloned into pBluescript plasmid digested with ClaI and XbaI. Following subcloning, the HA-Glut1 fragment is excised from pbluescript by BstXI and SaII digestion and directionally cloned into pBABE retrovirus expression vector digested with BstEI and SaII, thus generating the HA-GLUT1.

12.2 Retrovirus Production and Transduction

Retroviral stocks of the construct is produced using the method described in Pear et al. Proc. Natl. Acad Sci. U.S.A. 90: 8392-8396 1993. To generate C2C12 myoblast cells stably expressing the expression construct C2C12 were infected with the relevant virus for 3-5 h in the presence of 4 μg/ml Polybrene (Sigma). After a 48 h recovery period, infected cells are then selected in DMEM containing 10% FCS and supplemented with 2 μg/ml puromycin (Sigma).

Transduced myoblasts are seeded in proliferation medium (Dulbecco's modified Eagle's medium (DMEM) supplemented with 10% FCS) at a density of 12,000 cells per cm² and grown for 48 h to confluency. Cells are washed once with serum-free medium and induced to fuse in medium containing 2% horse serum (differentiation medium).

12.3 Analysis of Translocation of HA-GLUT1 in Differentiated C2C12 Cells

Retrovirally-transduced differentiated C2C12 cells expressing HA-tagged GLUT1 are subcultured for 30 hours. Insulin is then added at different time points, after which the cells are fixed in 3% formaldehyde. After quenching with 50 mM glycine, cells are incubated for 20 min with 5% normal swine serum (NSS) in the absence or presence of 0.1% saponin to analyse the level of HA-GLUT1 at the plasma membrane (PM) or the total cellular HA-GLUT1 content, respectively. Cells are incubated for 60 min with a saturating concentration of either an antibody directed against the HA tag or a control non-relevant antibody (mouse IgG MOPC21) in PBS containing 2% NSS. After extensive washing, the cells are incubated for 20 min with 5% NSS in the presence or absence of 0.1% saponin to permeabilize the cells. Cells are incubated for 60 min with saturating concentrations of ALEXA488-conjugated goat-anti-mouse antibody (20 μg/ml) and ALEXA594-conjugated WGA (10 μg/ml) in PBS containing 2% NSS. After washing, fluorescence (emm 485/exc 520 and emm 544/exc 630) is measured using the bottom-reading mode in a fluorescence microtiter plate reader (FLUOstar Galaxy, BMG Labtechnologies, Offenburg, Germany). The percentage of GLUT1 at the PM is calculated for each condition. ALEXA594-WGA fluorescence was used to correct for variation in cell density in each well. As a positive control a sample of cells are also incubated in the presence of

Dehydroepiandrosterone DHEA). DHEA has been previously shown to enhance levels of GLUT1 at the plasma membrane of a cell (Perini et al., Diabetes 53:41-52, 2004).

### EXAMPLE 13

### A Model to Determine the Effect of a CFTR Mutation on CFTR Translocation

The coding region of the CFTR gene (SEQ ID NO: 35) is isolated using methods essentially as described in Rommens et al., Proc. Natl. Acad. Sci. USA 88: 7500-7504, 1990. A double stranded oligonucleotide encoding HA tag is then inserted so as to encode the tag at the N terminus of the protein. The N-terminus of the CFTR is predicted to be an extracellular domain of the protein.

A vector comprising nucleic acid encoding the ΔF508 mutant of CFTh (SEQ ID NO: 62) is produced essentially as described in Tabacharani et al., Nature, 352: 628-632, 1991. The nucleic acid encoding the mutant CFIR is then modified to insert a double stranded oligonucleotide encoding HA tag is then inserted so as to encode the tag at the N terminus of the protein.

Each of the modified constructs is then cloned into the pBABE retroviral vector.

Retroviral stocks of each of the constructs are then produced using the method described in Pear et al. Proc. Natl Acad, Sci. U.S.A. 90: 8392-8396 1993. To generate COS cells stably expressing the expression construct COS were infected with the relevant virus for 3-5 h in the presence of 4 μg/ml Polybrene (Sigma). After a 48 h recovery period, infected cells are then selected in DMEM containing 10% FCS and supplemented with 2 μg/ml puromycin (Sigma).

The level of plasma membrane associated HA-CFTR or HA-CFIR-ΔF508 is then determined. Briefly, Retrovirally-transduced cells expressing HA-tagged CFTR or CFTR-ΔF508 are subcultured for 30 hours. Cells are then fixed in 3% formaldehyde. After quenching with 50 mM glycine, cells are incubated for 20 min with 5% normal 30 swine serum (NSS) in the absence or presence of 0.1% saponin to analyse the level of HA-labeled CFTR or mutant thereof at the plasma membrane (PM) or the total cellular HA-CPTR or CFTR-ΔF508 content, respectively. Cells are incubated for 60 min with a saturating concentration of either an antibody directed against the HA tag or a control non-relevant antibody in PBS containing 2% NSS. After extensive washing, the cells are incubated for 20 min with 5% NSS in the presence or absence of 0.1% saponin to permeabilize the cells. Cells are incubated for 60 min with saturating concentrations of ALEXA488-conjugated goat-anti-mouse antibody (20 μg/ml) and ALEXA594-conjugated WGA (10 μg/ml) in PBS containing 2% NSS. After washing, fluorescence (emm 485/exc 520 and emm 544/exc 630) is measured using the bottom-reading mode in a fluorescence microtiter plate reader (FLUOstar Galaxy, BMG Labtechnologies, Offenburg, Germany). The percentage of CFTR or CFTR-ΔF508 at the PM is calculated for each condition. ALEXA594WGA fluorescence was used to correct for variation in cell density in each well.

By comparing the level of HA-CFTR at the plasma membrane compared to the level of HA-CFTR-ΔF508 translocated to the plasma membrane, the effect of the ΔF508 mutation on translocation is determined.

1. A process for determining the level of a membrane transport protein translocated to the plasma membrane of a cell, said process comprising:
(a) determining the level of a membrane transport protein at the plasma membrane of the cell using a method comprising:
(i) contacting the cell with a ligand that binds to an extracellular domain of the membrane transport protein for a time and under conditions sufficient for the ligand to bind to the membrane transport protein at the plasma membrane of the cell; and
(ii) determining the level of ligand bound to the membrane transport protein;
(b) (i) permeabilizing or disrupting the plasma membrane of a cell and contacting the membrane transport protein within the cell with the ligand for a time and under conditions sufficient for the ligand to bind to the membrane transport protein; and
(ii) determining the level of ligand bound to the membrane transport protein; and
(c) comparing the level of ligand determined at (a) (ii) and (b) (ii) to determine the level of the membrane transport protein at the plasma membrane relative to the level of the membrane transport protein inside the cell.

2. The process according to claim 1 wherein the membrane transport protein is a glucose transport (GLUT) protein.

3. The process according to claim 2 wherein the membrane transport protein is GLUT4.

4. (canceled)

5. The process according to claim 2 wherein the membrane transport protein is GLUT1.

6. (canceled)

7. The process according to claim 1 wherein the membrane transport protein is a mutant membrane transport protein having a reduced rate of recycling or transporter internalization compared to a wild-type form of the membrane transport protein.

8. (canceled)

9. (canceled)

10. (canceled)

11. The process according to claim 1 wherein the membrane transport protein is labeled to facilitate binding of the ligand to the membrane transport protein.

12. (canceled)

13. (canceled)

14. The process according to claim 11 wherein the label comprises influenza virus hemagglutinin (HA) (SEQ ID NO: 15).

15. (canceled)

16. (canceled)

17. (canceled)

18. (canceled)

19. (canceled)

20. (canceled)

21. (canceled)

22. The process according to claim 1 wherein the ligand capable of binding to the membrane transport protein is an antibody.

23. (canceled)

24. (canceled)

25. (canceled)

26. (canceled)

27. The process according to claim 1 wherein the plasma membrane is permeablilized or disrupted by contacting the plasma membrane with an agent that permeabilizes or disrupts a membrane for a time and under conditions sufficient for permeabilization or disruption to occur.

28. (canceled)

29. (canceled)

30. The process according to claim 1 wherein the level of the ligand bound to the membrane transport protein is determined by a process comprising contacting the ligand with an antibody that specifically binds to the ligand for a time and under conditions sufficient for an antibody-antigen complex to form and determining the level of the complex wherein the level of the complex indicates the level of the ligand bound to the membrane transport protein.

31. (canceled)

32. The process according to claim 1 wherein the level of the ligand bound to the membrane transport protein is determined using a fluorescence linked immunosorbent assay.

33. The process according to claim 1 additionally comprising providing the cell expressing the membrane transport protein.

34. (canceled)

35. The process according to claim 1 additionally comprising fixing the cell.

36. (canceled)

37. (canceled)

38. (canceled)

39. The process according to claim 1 additionally comprising inducing translocation of the membrane transport protein to the plasma membrane.

40. (canceled)

41. (canceled)

42. (canceled)

43. The process according to claim 1 additionally comprising inducing resistance to translocation of the membrane transport protein in the cell.

44. (canceled)

45. (canceled)

46. (canceled)

47. The process of claim 1 comprising:
(a) determining the level of the membrane transport protein at the plasma membrane of a cell using a method comprising:
(i) contacting a cell with a ligand that binds to an extracellular domain of the membrane transport protein for a time and under conditions sufficient for the ligand to bind to the membrane transport protein; and
(ii) determining the level of ligand bound to the membrane transport protein;
(b) determining the level of the membrane transport protein within another cell using a method comprising:
(i) permeabilizing or disrupting the other cell;
(ii) contacting the membrane transport protein within the cell with the ligand for a time and under conditions sufficient for the ligand to bind the membrane transport protein;
(iii) determining the level of ligand bound to the membrane transport protein; and
(c) comparing the level of ligand detected at (a) (ii) and (b) (iii) to determine the level of the labeled membrane transport protein at the plasma membrane relative to the total level of labeled membrane transport protein.

48. The process according to claim 47 wherein the cells are isogenic or from the same cell line.

49. (canceled)

50. The process according to claim 47 or 48 wherein the cells are cultured under substantially similar conditions and wherein the level of the membrane transport protein at the plasma membrane of the cell and the level of membrane transport protein within the cell are each determined in a plurality of cells.

51. The process according to claim 50 additionally comprising normalizing the determined level of ligand bound to the membrane transport protein with regard to the number of cells in which the level of ligand bound to the membrane transport protein is determined.

52. (canceled)

53. (canceled)

54. A process for determining the level of a labeled GLUT4 protein or labeled mutant GLUT4 protein translocated to the plasma membrane of a cell, wherein said labeled mutant GLUT4 protein has a reduced rate of recycling or transporter internalization compared to a wild-type form of the membrane transport protein, said process comprising:
(a) determining the level of the labeled GLUT4 protein or labeled mutant GLUT4 protein at the plasma membrane of a cell expressing the labeled GLUT4 protein or labeled mutant GLUT4 protein using a method comprising:
(i) contacting the cell with a ligand that binds to the label for a time and under conditions sufficient for the ligand to bind to the labeled GLUT4 protein or labeled mutant GLUT4 protein; and
(ii) determining the level of ligand bound to the labeled GLUT4 protein or labeled mutant GLUT4 protein;
(b) determining the level of membrane transport protein within another cell expressing the labeled GLUT4 protein or labeled mutant GLUT4 protein using a method comprising:
(i) permeabilizing or disrupting the other cell;
(ii) contacting the labeled GLUT4 protein or labeled mutant GLUT4 protein within the cell with a ligand that binds to the label for a time and under conditions sufficient for the ligand to bind to the labeled GLUT4 protein or labeled mutant GLUT4 protein;
(iii) determining the level of ligand bound to the labeled GLUT4 protein or labeled mutant GLUT4 protein; and
(c) comparing the level of ligand detected at (a) (ii) and (b) (iii) to determine the level of the labeled GLUT4 protein or labeled mutant GLUT4 protein at the plasma membrane relative to the total level of labeled GLUT4 protein or labeled mutant GLUT4 protein.

55. A process for determining the level of a labeled GLUT4 protein or a labeled mutant GLUT4 protein translocated to the plasma membrane of a cell that is resistant to insulin induced GLUT4 translocation, wherein said labeled mutant GLUT4 protein has a reduced rate of recycling or transporter internalization compared to a wild-type form of the membrane transport protein, said process comprising:
(a) contacting a plurality of cells expressing a labeled GLUT4 protein or a labeled mutant GLUT4 protein with an amount of insulin sufficient to induce resistance to insulin induced translocation for a time and under conditions sufficient to induce resistance to insulin induced GLUT4 translocation in the cell, wherein the cells are contacted with insulin in the absence of serum and wherein the cells are contacted with insulin for a period of time from about 24 hours to about 48 hours;
(b) determining the level of the labeled GLUT4 protein or labeled mutant GLUT4 protein at the plasma membrane of a cell at (a) using a method comprising:
(i) contacting the cell with a ligand that binds to the label for a time and under conditions sufficient for the ligand to bind to the labeled GLUT4 protein or labeled mutant GLUT4 protein; and
(ii) determining the level of ligand bound to the labeled GLUT4 protein or labeled mutant GLUT4 protein;
(c) determining the level of labeled GLUT4 protein or labeled mutant GLUT4 protein in another cell at (a) but not (b) using a method comprising:
(i) permeabilizing or disrupting the other cell;
(ii) contacting the labeled GLUT4 protein or labeled mutant GLUT4 protein within the cell with a ligand that binds to the label for a time and under conditions sufficient for the ligand to bind to the labeled GLUT4 protein or labeled mutant GLUT4 protein;
(iii) determining the level of ligand bound to the labeled GLUT4 protein or labeled mutant GLUT4 protein; and
(d) comparing the level of ligand detected at (b) (ii) and (c) (iii) to determine the level of the labeled GLUT4 protein or labeled mutant GLUT4 protein at the plasma membrane relative to the total level of labeled GLUT4 protein or labeled mutant GLUT4 protein.

56. A process for determining the level of recycling of a membrane transport protein in a cell or a change in the level of recycling of a cell comprising:
(a) determining the level of the membrane transport protein translocated to the plasma membrane of a cell using the process according to claim 1;
(b) determining the level of the membrane transport protein translocated to the plasma membrane of another cell using the process according to claims 1, wherein the other cell is cultured for a longer period of time than the cell at (a); and
(c) comparing the level of the membrane transport protein translocated to the plasma membrane at (a) and (b) to thereby determine the level of recycling of the membrane transport protein in the cell, wherein a change in the level of the membrane transport protein translocated to the plasma membrane indicates a change in the level of recycling of a membrane transport protein.

57. A process for determining a mutation in a nucleic acid encoding a mutant membrane transport protein that is capable of modulating translocation of said membrane transport protein, said process comprising:
(a) determining the level of the mutant membrane transport protein translocated to the plasma membrane of a cell using the process according to claim 1; and
(b) determining the level of the wild-type form of the membrane transport protein translocated to the plasma membrane of a cell using the process according to claim 1,
wherein an enhanced or suppressed level of translocation of the membrane transport protein at (a) compared to (b) indicates that the nucleic acid comprises a mutation that is capable of modulating the level of level of translocation of the membrane transport protein to the plasma membrane.

58. A process for determining an agent that modulates translocation of a membrane transport protein to the plasma membrane of a cell, said process comprising:
(a) determining the level of a membrane transport protein translocated to the plasma membrane of a cell in the absence of a candidate agent by performing the process according to claim 1;
(b) determining the level of the membrane transport protein translocated to the plasma membrane of a cell in the presence of the candidate agent by performing the process according to claim 1, wherein a difference in the level of the membrane transport protein translocated to the plasma membrane of a cell at (a) compared to (b) indicates that the candidate agent modulates translocation of the membrane transport protein.
(c) optionally, determining the structure of the candidate agent;
(d) optionally, providing the name or structure of the candidate agent; and
(e) optionally, providing, the candidate agent.

59. A process for determining a candidate compound for the treatment of insulin resistance comprising:
(a) determining the level of the labeled GLUT4 protein or the labeled mutant GLUT4 protein translocated to the plasma membrane of a cell in the absence of a candidate agent by performing the process according to claim 55, wherein said labeled mutant GLUT4 protein has a reduced rate of recycling or transporter internalization compared to a wild-type form of the membrane transport protein; and
(b) determining the level of the labeled GLUT4 protein or a labeled mutant GLUT4 protein translocated to the plasma membrane of another cell in the presence of the candidate agent by performing the process according to claim 55, wherein said labeled mutant GLUT4 protein has a reduced rate of recycling or transporter internalization compared to a wild-type form of the membrane transport protein and wherein a candidate agent that enhances the level of translocation of the labeled GLUT4 protein or a labeled mutant GLUT4 protein is a candidate agent for the treatment of insulin resistance.
(c) optionally, determining the structure of the candidate agent;
(d) optionally, providing the name or structure of the candidate agent; and
(e) optionally, providing, the candidate agent.

60. (canceled)

61. (canceled)

62. A process for manufacturing a medicament for the treatment of insulin resistance comprising:
(a) determining a candidate agent for the treatment of insulin resistance using a process comprising:
(i) determining the level of the labeled GLUT4 protein or the labeled mutant GLUT4 protein translocated to the plasma membrane of a cell in the absence of a candidate agent by performing the process according to claim 55, wherein said labeled mutant GLUT4 protein has a reduced rate of recycling or transporter internalization compared to a wild-type form of the membrane transport protein; and
(ii) determining the level of the labeled GLUT4 protein or a labeled mutant GLUT4 protein translocated to the plasma membrane of another cell in the presence of the candidate agent by performing the process according to claim 55, wherein said labeled mutant GLUT4 protein has a reduced rate of recycling or transporter internalization compared to a wild-type form of the membrane transport protein and wherein a candidate agent that enhances the level of translocation of the labeled GLUT4 protein or a labeled mutant GLUT4 protein is a candidate agent for the treatment of insulin resistance.
(b) optionally, isolating the candidate agent;
(c) optionally, providing the name or structure of the candidate agent;
(d) optionally, providing the candidate agent; and
